SEQUENCE LISTING (148 sequences)

SEQ ID NO: 1 - DNA, 30 bp, synthetic construct
  misc_feature 1..30: note = Adenovirus pIX fragment nucleotides
  source 1..30: mol_type = other DNA
agctctttggtggcgagcggcgcggcctct

SEQ ID NO: 2 - DNA, 439 bp, synthetic construct
  misc_feature 1..439: note = Adenovirus DNA polymerase fragment nucleotides
  source 1..439: mol_type = other DNA
aacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgc

SEQ ID NO: 3 - DNA, 34 bp, synthetic construct
  misc_feature 1..34: note = Adenovirus penton base protein fragment nucleotides
  source 1..34: mol_type = other DNA
actgaggctgcggctaaggctgaggtcgaagcca

SEQ ID NO: 4 - DNA, 45 bp, synthetic construct
  misc_feature 1..45: note = Adenovirus hexon protein fragment nucleotides
  source 1..45: mol_type = other DNA
ataggtgtggatgccacacaggcgggagataaccctatatatgct

SEQ ID NO: 5 - DNA, 30 bp, synthetic construct
  misc_feature 1..30: note = Adenovirus fiber protein fragment nucleotides
  source 1..30: mol_type = other DNA
gtagcaggccccctagctgtggccaatggc

SEQ ID NO: 6 - DNA, 399 bp, synthetic construct
  misc_feature 1..399: note = Adenovirus pIX nucleotides
  source 1..399: mol_type = other DNA
atgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccc

SEQ ID NO: 7 - DNA, 3168 bp, synthetic construct
  misc_feature 1..3168: note = Adenovirus DNA polymerase nucleotides
  source 1..3168: mol_type = other DNA
atggacagctccaatgtgcgcgatgtcgtcatcaaactccgcccgccgagcgccgagatctggacctgcggctctcgcggcgtggtggtctgctccaccatcgccctccaggagacagatgctggcggccagacaaccaaagtagaagaccaccagccacacgggaccccaggcgggggacttagattcccgctgcgcttcctcgtcagaggtcgccaggttcacctcgtgcaagatatacaacccgtgcagcgctgccagtactgcggtcgcttttacaaaagccagcacgagtgctcggcccgcagacgggacttctactttcaccacatcaacagccaatcctccaactggtggcgggagatccagttcttcccgatcggctctcatcctcgcacggagcgcctctttgtcacctacgatgtagagacctacacttggatgggagcctttggcaagcagctcgtgcccttcatgctggtcatgaaactggggggcgacgaggctctggtcgccgccgcgcgcgacctcgcccgagagctcagatgggacccctgggagaaagaccccctcaccttctactgcatcacccccgaaaagatggccgtggggcgacagttcagaaccttccgcgaccgcctgcagaccctcatggcccgcgacctctggcgatccttcctggcggccaaccctcacttgcaagactgggccctggaggagcacggcctggaatcgcccgaggagctcacctacgaggaactcaaaaagctcccctccatcaagggccagccccgctttttggagctctacatcgtgggccacaacataaacggctttgacgagatcgtcctggccgcccaggtcatcaacaaccgctcctcggtcccagggccctttcgcatcaccagaaacttcatgcctcgagcggggaagatcctcttcaatgacctcaccttctccctgcccaacccgcgctccaaaaagcgcacggactacaccctgtgggaacagggcggctgcgatgacacagacttcaaacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgccttccgcgccgagcgcccccagcgcaccaacctcgggcccaacatgctggccccctcccacgagctctatgactacgtgcgcgccagcatccgcggggggcgctgctaccccacctacctcggcatcctcagggaacccctgtacgtgtatgacatctgcggcatgtacgcctccgcgctcacccaccccatgccctggggcccgcccctcaacccctacgagcgcgcgctcgccgcccgcgaatggcagcgggctctggacatgcaagcttgcaagatcgactactttgacccgcgcttgctccccggggtcttcaccatcgacgcggaccccccaaacgaggaccagctggaccccctaccccccttctgctcgcgcaagggcggccgcctctgctggaccaacgagcgcctgcgcggcgaggtcgccaccagcgtcgacatggtcaccctgcacaaccgaggctggagggtgcgcctaatcccagacgagcgcaccaccgtcttccccgagtggaagtgcgtggcccgcgagtacgtgcaactcaacatcgcggccaaggagcgagccgaccgcgacaaaaaccagaccctgcgctccatcgccaagctgctctccaacgccctctacgggtcgttcgccaccaagcttgacaacaaaaaaatagtgttttctgaccagatggacccaggtaccctcaaaggtatcacctccggacaggtgaacatcaaatcctcctcatttttagaaactgacaacctgagcgctgaggtcatgcccgccttcgagagggaatacttaccccagcagctggccctcgcagacagcgatgcggaagagagtgaagatgaaagggcgcccacccccttttatacccccccgtcgggaacccccggtcacgtgtcctacacctacaagccaatcacttttctggacgcggaggagggggacatgtgcctgcacaccctggagaaggtggacccgctagtggacaacgaccgctacccctcccacgtggcctccttcgtcctggcctggacgcgggccttcgtctcagagtggtcagagtttctctacgaggaggacagaggcactccgctggaagacaggcccctgaagtcggtctacggggacacggacagcctcttcgtcaccgagaagggacaccgcctcatggagagccgaggtaagaaacgcatcaaaaagcatgggggcaacctggtttttgaccctgaccgcccggagctcacttggctggtggaatgcgagacggtctgcgcttcctgcggcgcggacgcctactccccagagtccgtgtttctcgctcccaagctctacgccctgaagagcctgcagtgcccctcgtgcggcgccacctccaagggaaagctccgcgccaaggggcacgccgccgagggtctcgactacgagaccatggtcaaatgctacctggccgacgcgcagggcgaagagcggcagcgattcagcaccagcagaaccagcctcaagcgcaccctggccagcgcccaacccggagcgcaccccttcaccgtgacccagaccaccctgacgaggaccctgcgcccatggaaggacatgactctggccccgctggacgcccatcggctggtgccctacagcgaaagccgccccaacccgcgaaacgaggagatctgctggatcgagatgccg

SEQ ID NO: 8 - DNA, 1974 bp, synthetic construct
  misc_feature 1..1974: note = Adenovirus penton base protein nucleotides
  source 1..1974: mol_type = other DNA
atgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttt

SEQ ID NO: 9 - DNA, 2877 bp, synthetic construct
  misc_feature 1..2877: note = Adenovirus hexon protein nucleotides
  source 1..2877: mol_type = other DNA
atggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacc

SEQ ID NO: 10 - DNA, 1749 bp, synthetic construct
  misc_feature 1..1749: note = Adenovirus fiber protein nucleotides
  source 1..1749: mol_type = other DNA
atgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaa

SEQ ID NO: 11 - protein, 10 aa, synthetic construct
  REGION 1..10: note = Adenovirus pIX protein fragment
SSLVASGAAS

SEQ ID NO: 12 - protein, 147 aa, synthetic construct
  REGION 1..147: note = Adenovirus DNA polymerase fragment
KHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIA

SEQ ID NO: 13 - protein, 15 aa, synthetic construct
  REGION 1..15: note = Adenovirus penton base protein fragment
KTEAAAKAEVEANVA

SEQ ID NO: 14 - protein, 15 aa, synthetic construct
  REGION 1..15: note = Adenovirus hexon protein fragment
IGVDATQAGDNPIYA

SEQ ID NO: 15 - protein, 15 aa, synthetic construct
  REGION 1..15: note = Adenovirus fiber protein fragment
LNVAGPLAVANGINA

SEQ ID NO: 16 - protein, 133 aa, synthetic construct
  REGION 1..133: note = Adenovirus pIX amino acids
MSDTGNSFDGSIFSPYLTVRMPHWAGVRQNVMGSNVDGRPVLPSNSSTMAYATVGGTPLDAATSAAASAAAATARSMATDLYSSLVASGAASRASARDEKLTALLLKLEDLTRELGQLTQQVSSLRESSLASP

SEQ ID NO: 17 - protein, 1056 aa, synthetic construct
  REGION 1..1056: note = Adenovirus DNA polymerase amino acids
MDSSNVRDVVIKLRPPSAEIWTCGSRGVVVCSTIALQETDAGGQTTKVEDHQPHGTPGGGLRFPLRFLVRGRQVHLVQDIQPVQRCQYCGRFYKSQHECSARRRDFYFHHINSQSSNWWREIQFFPIGSHPRTERLFVTYDVETYTWMGAFGKQLVPFMLVMKLGGDEALVAAARDLARELRWDPWEKDPLTFYCITPEKMAVGRQFRTFRDRLQTLMARDLWRSFLAANPHLQDWALEEHGLESPEELTYEELKKLPSIKGQPRFLELYIVGHNINGFDEIVLAAQVINNRSSVPGPFRITRNFMPRAGKILFNDLTFSLPNPRSKKRTDYTLWEQGGCDDTDFKHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIAFRAERPQRTNLGPNMLAPSHELYDYVRASIRGGRCYPTYLGILREPLYVYDICGMYASALTHPMPWGPPLNPYERALAAREWQRALDMQACKIDYFDPRLLPGVFTIDADPPNEDQLDPLPPFCSRKGGRLCWTNERLRGEVATSVDMVTLHNRGWRVRLIPDERTTVFPEWKCVAREYVQLNIAAKERADRDKNQTLRSIAKLLSNALYGSFATKLDNKKIVFSDQMDPGTLKGITSGQVNIKSSSFLETDNLSAEVMPAFEREYLPQQLALADSDAEESEDERAPTPFYTPPSGTPGHVSYTYKPITFLDAEEGDMCLHTLEKVDPLVDNDRYPSHVASFVLAWTRAFVSEWSEFLYEEDRGTPLEDRPLKSVYGDTDSLFVTEKGHRLMESRGKKRIKKHGGNLVFDPDRPELTWLVECETVCASCGADAYSPESVFLAPKLYALKSLQCPSCGATSKGKLRAKGHAAEGLDYETMVKCYLADAQGEERQRFSTSRTSLKRTLASAQPGAHPFTVTQTTLTRTLRPWKDMTLAPLDAHRLVPYSESRPNPRNEEICWIEMP

SEQ ID NO: 18 - protein, 658 aa, synthetic construct
  REGION 1..658: note = Adenovirus penton base protein
MRRAAMFEEGPPPSYESAMGISPAAPLQPPYVPPRYLQPTGGRNSICYSELQPLYDTTRLYLVDNKSADVASLNYQNDHSDFLTTVIQNNDFTPTEASTQTINLDNRSNWGGDLKTILHTNMPNVNEFMFTNSFKARVMVAREQGEAKYEWVDFTLPEGNYSETMTLDLMNNAIVEHYLKVGRQNGVKESDIGVKFDTRNFRLGWDPVTGLVMPGVYTNEAFHPDIVLLPGCGVDFTQSRLSNLLGIRKRQPFQEGFKITYEDLKGGNIPALLDLDAYEESLKPEESAGDSGESGEEQAGGGGGASVENESTPAVAADAAEVEPEAMQQDAEEGAQEGAQKDMNDGEIRGDTFATRGEEKEAEAAAAATAEAETEVEAEAEPETEVMEDMNDGERRGDTFATRGEEKAAEAEAAAEEAAAAAAKTEAAAKAEVEANVAVEAQAEEEAAAEAVKEKAQAEQEEKKPVIQPLKEDSKKRSYNVIEGSTFTQYRSWYLAYNYGDPVKGVRSWTLLCTPDVTCGSEQMYWSLPNMMQDPVTFRSTRQVSNFPVVGAELLPVHSKSFYNEQAVYSQLIRQATSLTHVFNRFPENQILARPPAPTITTVSENVPALTDHGTLPLRNSISGVQRVTITDARRRTCPYVYKALGIVSPRVLSSRTF

SEQ ID NO: 19 - protein, 959 aa, synthetic construct
  REGION 1..959: note = Adenovirus hexon protein
MATPSMMPQWSYMHISGQDASEYLSPGLVQFARATDTYFNMSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQDEPAQAAIAEDEEELEEEQAQDEQAPTKKTHVYAQAPLSGEKITKDGLQIGVDATQAGDNPIYADKTFQPEPQIGESQWNEADATVAGGRVLKKTTPMRPCYGSYAKPTNANGGQGIMVANDQGALESKVEMQFFSTTTSLNVREGENNLQPKVVLYSEDVNLESPDTHLSYKPKKDDTNSKIMLGQQAMPNRPNLIAFRDNFIGLMYYNSTGNMGVLAGQASQLNAVVDLQDRNTELSYQLMLDSIGDRSRYFSMWNQAVDSYDPDVRIIENHGVEDELPNYCFPLGGIGITDTYQCIKPTAAANNTTWSKDEEFSDRNEIGVGNNFAMEINIQANLWRNFLYANVGLYLPDKLKYNPTNVDISDNPNTYDYMNKRVVAPGLVDCFVNVGARWSLDYMDNVNPFNHHRNAGLRYRSMILGNGRYVPFHIQVPQKFFAIKNLLLLPGSYTYEWNFRKDVNMVLQSSLGNDLRVDGASIKFDSVTLYATFFPMAHNTASTLEAMLRNDTNDQSFNDYLSGANMLYPIPAKATNVPISIPSRNWAAFRGWAFTRLKTKETPSLGSGFDPYFVYSGSIPYLDGTFYLNHTFKKISIMYDSSVSWPGNDRLLTPNEFEVKRAVDGEGYNVAQCNMTKDWFLVQMLANYNIGYQGFYIPESYKDRMYSFFRNFQPMSRQVVDETKYKDYQAIGITHQHNNSGFVGYLAPTMREGQAYPANFPYPLIGKTAVDSVTQKKFLCDRTLWRIPFSSNFMSMGALTDLGQNLLYANSAHALDMTFEVDPMDEPTLLYIVFEVFDVVRVHQPHRGVIETVYLRTPFSAGNATT

SEQ ID NO: 20 - protein, 583 aa, synthetic construct
  REGION 1..583: note = Adenovirus fiber protein
MKRARSSDETFNPVYPYDTEIAPTSVPFLTPPFVSSAGMQENPAGVLSLHLSEPLTTHNGALTLKMGGGLTLDKEGNLTSQNITSVDPPLKKSKNNISLQTAAPLAVSSGALTLFATPPLAVSGDNLTVQSQAPLTLEDSKLTLATKGPLTVSEGKLVLETEAPLHASDSSSLGLSVTAPLSINNDSLGLDLQAPIVSQNGKLALNVAGPLAVANGINALTVGTGKGIGLNETSTHLQAKLVAPLGFDTNGNIKLSVAGGMRLNNDTLILDVNYPFEAQGQLSLRVGQGPLYVDSSSHNLTIRCLRGLYITSSNNQTGLEANIKLTKGLVYDGNAIAVNVGQGLQYSTTATSEGVYPIQSKIGLGMEYDTNGAMMTKLGSGLSFDNSGAIVVGNKNDDRLTLWTTPDPSPNCRIYSEKDTKLTLVLTKCGSQILGTVSALAVRGSLAPITNASSIVQIFLRFDENGLLMSNSSLDGDYWNYRNGDSTNSTPYTNAVGFMPNLAAYPKGQATAAKSSIVSQVYMDGDTTKPITLKINFNGIDETTENTPVSKYSMTFSWSWPTASYIGHTFATNSFTFSYIAQE

SEQ ID NO: 21 - DNA, 32739 bp, synthetic construct
  misc_feature 1..32739: note = Adenovirus vector nucleotide sequences
  source 1..32739: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacaga

SEQ ID NO: 22 - DNA, 32739 bp, synthetic construct
  misc_feature 1..32739: note = Adenovirus vector nucleotide sequences
  source 1..32739: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaac

SEQ ID NO: 23 - DNA, 32739 bp, synthetic construct
  misc_feature 1..32739: note = Adenovirus vector nucleotide sequences
  source 1..32739: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccc

SEQ ID NO: 24 - DNA, 32739 bp, synthetic construct
  misc_feature 1..32739: note = Adenovirus vector nucleotide sequences
  source 1..32739: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctc

SEQ ID NO: 25 - DNA, 32739 bp, synthetic construct
  misc_feature 1..32739: note = Adenovirus vector nucleotide sequences
  source 1..32739: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacgcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaataaagaaagcacagagatgcttgttttgatttcaaaattgtgtgcttttatttattttcagcttacagtatttccagtagtcattcgaataaagcttaatcaaactgcatgagaacccttccacatagcttaaattagcaccagtgcaaatggagaaaagcctcgaggtcgttgcgcggccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattac

SEQ ID NO: 26 - DNA, 114 bp, synthetic construct
  misc_feature 1..114: note = IL-2 core promoter
  source 1..114: mol_type = other DNA
acattttgacacccccataatatttttccagaattaacagtataaattgcatctcttgttcaagagttccctatcactctctttaatcactactcacagtaacctcaactcctg

SEQ ID NO: 27 - DNA, 55 bp, synthetic construct
  misc_feature 1..55: note = IL-2 minimal promoter
  source 1..55: mol_type = other DNA
tcaagagttccctatcactctctttaatcactactcacagtaacctcaactcctg

SEQ ID NO: 28 - DNA, 380 bp, synthetic construct
  misc_feature 1..380: note = IL-2 enhancer and promoter variant
  source 1..380: mol_type = other DNA
tgatatcttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 29 - DNA, 373 bp, synthetic construct
  misc_feature 1..373: note = L-2 enhancer and promoter variant
  source 1..373: mol_type = other DNA
ttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 30 - DNA, 161 bp, synthetic construct
  misc_feature 1..161: note = (NF-KB)1-IL2 promoter variant
  source 1..161: mol_type = other DNA
aattggtcccatcgaagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 31 - DNA, 201 bp, synthetic construct
  misc_feature 1..201: note = (NF-KB)3-IL2 promoter variant
  source 1..201: mol_type = other DNA
aattggtcccatcgaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 32 - DNA, 268 bp, synthetic construct
  misc_feature 1..268: note = (NF-kB)6-IL2 promoter variant
  source 1..268: mol_type = other DNA
aattggtcccatcgaagagggatttcacctacataagagggatttcacctacataagagggatttcacctacataattggtaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 33 - DNA, 177 bp, synthetic construct
  misc_feature 1..177: note = 1X NFAT response elements-IL2 promoter variant
  source 1..177: mol_type = other DNA
aattggtcccatcgaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 34 - DNA, 256 bp, synthetic construct
  misc_feature 1..256: note = 3X NFAT response elements-IL2 promoter variant
  source 1..256: mol_type = other DNA
tgatatcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 35 - DNA, 249 bp, synthetic construct
  misc_feature 1..249: note = 3X NFAT response elements-IL2 promoter variant
  source 1..249: mol_type = other DNA
aattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 36 - DNA, 358 bp, synthetic construct
  misc_feature 1..358: note = 6X NFAT response elements-IL2 promoter variant
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctccttaatcactactcacagtaacctcaactcctg

SEQ ID NO: 37 - DNA, 374 bp, synthetic construct
  misc_feature 1..374: note = 6X NFAT response elements-IL2 promoter variant
  source 1..374: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgaattccatg

SEQ ID NO: 38 - DNA, 358 bp, synthetic construct
  misc_feature 1..358: note = 6X NFAT response elements-IL2 promoter variant
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 39 - DNA, 365 bp, synthetic construct
  misc_feature 1..365: note = 6X NFAT response elements-IL2 promoter variant
  source 1..365: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 40 - DNA, 244 bp, synthetic construct
  misc_feature 1..244: note = human EEF1A1 promoter variant
  source 1..244: mol_type = other DNA
gagcgtgcgtgaggctccggtgcccgtcagtgggcagagcgcacatcgcccacagtccccgagaagttggggggagggggtcggcgattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 41 - DNA, 236 bp, synthetic construct
  misc_feature 1..236: note = human EEF1A1 promoter variant
  source 1..236: mol_type = other DNA
gcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 42 - DNA, 1266 bp, synthetic construct
  misc_feature 1..1266: note = human EEF1A1 promoter and enhancer
  source 1..1266: mol_type = other DNA
gagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 43 - DNA, 571 bp, synthetic construct
  misc_feature 1..571: note = human UBC promoter
  source 1..571: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacg

SEQ ID NO: 44 - DNA, 81 bp, synthetic construct
  misc_feature 1..81: note = synthetic minimal promoter 1
  source 1..81: mol_type = other DNA
aggtctatataagcagagctcgtttagtgaaccctcattctggagacggatcccgagccgagtgttttgacctccatagaa

SEQ ID NO: 45 - protein, 10 aa, synthetic construct
  REGION 1..10: note = HBV Peptides from Core 1
FLPSDFFPSV

SEQ ID NO: 46 - protein, 8 aa, synthetic construct
  REGION 1..8: note = HBV Peptides from Core 2
CWGELMTL

SEQ ID NO: 47 - protein, 9 aa, synthetic construct
  REGION 1..9: note = HBV Peptides from Core 3
GVWIRTPPA

SEQ ID NO: 48 - protein, 11 aa, synthetic construct
  REGION 1..11: note = HBV Peptides from Core 4
STLPETTVVRR

SEQ ID NO: 49 - protein, 11 aa, synthetic construct
  REGION 1..11: note = HBV Peptides from Core 5
LTFGRETVLEY

SEQ ID NO: 50 - protein, 10 aa, synthetic construct
  REGION 1..10: note = HBV Peptides from Core 6
DLLDTASALY

SEQ ID NO: 51 - protein, 10 aa, synthetic construct
  REGION 1..10: note = HBV Peptides from Core 7
LWFHISCLTF

SEQ ID NO: 52 - protein, 9 aa, synthetic construct
  REGION 1..9: note = HBV Peptides from Core 8
EYLVSFGVW

SEQ ID NO: 53 - protein, 9 aa, synthetic construct
  REGION 1..9: note = HBV Peptides from Polymerase 1
FLKQQYMNL

SEQ ID NO: 54 - protein, 9 aa, synthetic construct
  REGION 1..9: note = HBV Peptides from Polymerase 2
FLSKQYMDL

SEQ ID NO: 55 - protein, 9 aa, synthetic construct
  REGION 1..9: note = HBV Peptides from Polymerase 3
TVSTKLCKI

SEQ ID NO: 56 - protein, 9 aa, synthetic construct
  REGION 1..9: note = HBV Peptides from Polymerase 4
GLSRYVARL

SEQ ID NO: 57 - protein, 9 aa, synthetic construct
  REGION 1..9: note = HBV Peptides from Polymerase 5
KLHLYSHPI

SEQ ID NO: 58 - protein, 9 aa, synthetic construct
  REGION 1..9: note = HBV Peptides from Polymerase 6
FLLSLGIHL

SEQ ID NO: 59 - protein, 9 aa, synthetic construct
  REGION 1..9: note = HBV Peptides from Polymerase 7
SLYADSPSV

SEQ ID NO: 60 - protein, 9 aa, synthetic construct
  REGION 1..9: note = HBV Peptides from Polymerase 8
ALMPLYACI

SEQ ID NO: 61 - DNA, 10410 bp, synthetic construct
  misc_feature 1..10410: note = GCAd-RTS-IL12 design 1
  source 1..10410: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataatgcaggtcggagtactgtcctccgagcggagtactgtcctccgagcggagtactgtcctccgagcggagtactgtcctccgagcggagtactgtcctccgagcggagtactgtcctccgagcggagactcttcgaaggaagaggggcggggtcgatcgaccccgcccctcttccttcgaaggaagaggggcggggtcgaagacctagagggtatataatgggtgccttagctggtgtgtgagctcatcttcctgtagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttgggcgagctcgaattcattgatcccccgggctgcaggaattcgatatcaagctcgggatccgaattccgccccccccccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaacgtacgtcgacatcgagaacttgtttattgcagcttataatggttacaaataaagcaatagcatcacaaatttcacaaataaagcatttttttcactgcattctagttgtggtttgtccaaactcatcaatgtatcttatcatgtctgggcgcgccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaggcggcctctagagcggccgccaccgcggggagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 62 - DNA, 10040 bp, synthetic construct
  misc_feature 1..10040: note = GCAd-RTS-IL12 design 2
  source 1..10040: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtctcgagggcggcggagagggcagaggaagtcttctaacatgcggtgacgtggaggagaatcccggccctaggatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 63 - DNA, 9949 bp, synthetic construct
  misc_feature 1..9949: note = GCAd-RTS-IL12 design 3
  source 1..9949: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggcagccgctaaatccaaggtaaggtcagaagagctagcgccaccatgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctgacatgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgacattctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 64 - DNA, 54 bp, synthetic construct
  misc_feature 1..54: note = Whitlow Linker
  source 1..54: mol_type = other DNA
ggcagcacctccggcagcggcaagcctggcagcggcgagggcagcaccaagggc

SEQ ID NO: 65 - DNA, 78 bp, synthetic construct
  misc_feature 1..78: note = Linker
  source 1..78: mol_type = other DNA
tctggcggaggatctggaggaggcggatctggaggaggaggcagtggaggcggaggatctggcggaggatctctgcag

SEQ ID NO: 66 - skipped

SEQ ID NO: 67 - DNA, 12 bp, synthetic construct
  misc_feature 1..12: note = SGSG linker
  source 1..12: mol_type = other DNA
agtggcagcggc

SEQ ID NO: 68 - DNA, 45 bp, synthetic construct
  misc_feature 1..45: note = (G4S)3 linker
  source 1..45: mol_type = other DNA
ggtggcggtggctcgggcggtggtgggtcgggtggcggcggatct

SEQ ID NO: 69 - DNA, 12 bp, synthetic construct
  misc_feature 1..12: note = Furin cleavage site
  source 1..12: mol_type = other DNA
cgtgcaaagcgt

SEQ ID NO: 70 - DNA, 84 bp, synthetic construct
  misc_feature 1..84: note = Fmdv
  source 1..84: mol_type = other DNA
agagccaagagggcaccggtgaaacagactttgaattttgaccttctgaagttggcaggagacgttgagtccaaccctgggccc

SEQ ID NO: 71 - DNA, 54 bp, synthetic construct
  misc_feature 1..54: note = Thosea asigna virus 2A region (T2A)
  source 1..54: mol_type = other DNA
gagggcagaggaagtctgctaacatgcggtgacgtcgaggagaatcctggacct

SEQ ID NO: 72 - DNA, 75 bp, synthetic construct
  misc_feature 1..75: note = Furin-GSG-T2A
  source 1..75: mol_type = other DNA
agagctaagaggggaagcggagagggcagaggaagtctgctaacatgcggtgacgtcgaggagaatcctggacct

SEQ ID NO: 73 - DNA, 78 bp, synthetic construct
  misc_feature 1..78: note = Furin-SGSG-T2A
  source 1..78: mol_type = other DNA
agggccaagaggagtggcagcggcgagggcagaggaagtcttctaacatgcggtgacgtggaggagaatcccggccct

SEQ ID NO: 74 - DNA, 57 bp, synthetic construct
  misc_feature 1..57: note = Porcine teschovirus-1 2A region (P2A)
  source 1..57: mol_type = other DNA
gcaacgaacttctctctcctaaaacaggctggtgatgtggaggagaatcctggtcca

SEQ ID NO: 75 - DNA, 66 bp, synthetic construct
  misc_feature 1..66: note = GSG-P2A
  source 1..66: mol_type = other DNA
ggaagcggagctactaacttcagcctgctgaagcaggctggagacgtggaggagaaccctggacct

SEQ ID NO: 76 - DNA, 60 bp, synthetic construct
  misc_feature 1..60: note = Equine rhinitis A virus 2A region (E2A)
  source 1..60: mol_type = other DNA
cagtgtactaattatgctctcttgaaattggctggagatgttgagagcaaccctggacct

SEQ ID NO: 77 - DNA, 66 bp, synthetic construct
  misc_feature 1..66: note = Foot-and-mouth disease virus 2A region (F2A)
  source 1..66: mol_type = other DNA
gtcaaacagaccctaaactttgatctgctaaaactggccggggatgtggaaagtaatcccggcccc

SEQ ID NO: 78 - DNA, 93 bp, synthetic construct
  misc_feature 1..93: note = FP2A
  source 1..93: mol_type = other DNA
cgtgcaaagcgtgcaccggtgaaacagggaagcggagctactaacttcagcctgctgaagcaggctggagacgtggaggagaaccctggacct

SEQ ID NO: 79 - DNA, 24 bp, synthetic construct
  misc_feature 1..24: note = Linker-GSG
  source 1..24: mol_type = other DNA
gcaccggtgaaacagggaagcgga

SEQ ID NO: 80 - DNA, 15 bp, synthetic construct
  misc_feature 1..15: note = Linker
  source 1..15: mol_type = other DNA
gcaccggtgaaacag

SEQ ID NO: 81 - protein, 18 aa, synthetic construct
  REGION 1..18: note = Whitlow Linker Amino Acid Sequence
GSTSGSGKPGSGEGSTKG

SEQ ID NO: 82 - protein, 26 aa, synthetic construct
  REGION 1..26: note = Linker Amino Acid Sequence
SGGGSGGGGSGGGGSGGGGSGGGSLQ

SEQ ID NO: 83 - skipped

SEQ ID NO: 84 - protein, 4 aa, synthetic construct
  REGION 1..4: note = SGSG Linker Amino Acid Sequence
SGSG

SEQ ID NO: 85 - protein, 15 aa, synthetic construct
  REGION 1..15: note = G4S LINKER Amino Acid Sequence
GGGGSGGGGSGGGGS

SEQ ID NO: 86 - protein, 4 aa, synthetic construct
  REGION 1..4: note = Furin Cleavage site Amino Acid Sequence
RAKR

SEQ ID NO: 87 - protein, 28 aa, synthetic construct
  REGION 1..28: note = FMDV Amino Acid Sequence
RAKRAPVKQTLNFDLLKLAGDVESNPGP

SEQ ID NO: 88 - protein, 18 aa, synthetic construct
  REGION 1..18: note = Thosea asigna virus 2a Region (T2A) Amino Acid Sequence
EGRGSLLTCGDVEENPGP

SEQ ID NO: 89 - protein, 25 aa, synthetic construct
  REGION 1..25: note = FURIN-SGSG-T2A Amino Acid Sequence
RAKRGSGEGRGSLLTCGDVEENPGP

SEQ ID NO: 90 - protein, 26 aa, synthetic construct
  REGION 1..26: note = FURIN-SGSG-T2A Amino Acid Sequence
RAKRSGSGEGRGSLLTCGDVEENPGP

SEQ ID NO: 91 - protein, 19 aa, synthetic construct
  REGION 1..19: note = Porcine teschovirus-1 2A region (P2A) Amino Acid Sequence
ATNFSLLKQAGDVEENPGP

SEQ ID NO: 92 - protein, 22 aa, synthetic construct
  REGION 1..22: note = GSG-P2A Amino Acid Sequence
GSGATNFSLLKQAGDVEENPGP

SEQ ID NO: 93 - protein, 20 aa, synthetic construct
  REGION 1..20: note = GSG-P2A Amino Acid Sequence
QCTNYALLKLAGDVESNPGP

SEQ ID NO: 94 - protein, 22 aa, synthetic construct
  REGION 1..22: note = Foot-and-motuh disease virus 2A region (F2A) Amino Acid Sequence
VKQTLNFDLLKLAGDVESNPGP

SEQ ID NO: 95 - protein, 31 aa, synthetic construct
  REGION 1..31: note = FP2A Amino Acid Sequence
RAKRAPVKQGSGATNFSLLKQAGDVEENPGP

SEQ ID NO: 96 - protein, 8 aa, synthetic construct
  REGION 1..8: note = LINKER-GSG Amino Acid Sequence
APVKQGSG

SEQ ID NO: 97 - protein, 5 aa, synthetic construct
  REGION 1..5: note = LINKER Amino Acid Sequence
APVKQ

SEQ ID NO: 98 - protein, 154 aa, synthetic construct
  REGION 1..154: note = HBV HBx domain of HBV design 1
MAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSA

SEQ ID NO: 99 - protein, 454 aa, synthetic construct
  REGION 1..454: note = HBV Pol domain of HBV design 1
GPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 100 - protein, 226 aa, synthetic construct
  REGION 1..226: note = HBV Surface (Env1) domain of HBV domain 1
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYI

SEQ ID NO: 101 - protein, 180 aa, synthetic construct
  REGION 1..180: note = HBV Core domain of HBV design 1
MQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQ

SEQ ID NO: 102 - protein, 226 aa, synthetic construct; identical to SEQ ID NO: 100
  REGION 1..226: note = SHB(Env) domain

SEQ ID NO: 103 - protein, 180 aa, synthetic construct; identical to SEQ ID NO: 101
  REGION 1..180: note = HBeAg domain

SEQ ID NO: 104 - protein, 154 aa, synthetic construct; identical to SEQ ID NO: 98
  REGION 1..154: note = HBx domain

SEQ ID NO: 105 - protein, 454 aa, synthetic construct; identical to SEQ ID NO: 99
  REGION 1..454: note = Pol domain

SEQ ID NO: 106 - protein, 1014 aa, synthetic construct
  REGION 1..1014: note = HBV design 1
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYIMQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQMAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSAGPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 107 - protein, 1021 aa, synthetic construct
  REGION 1..1021: note = HBV design 2
SVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQRGRTIVLHKRTLGLMGQNLSTSNPLGFFPDHQLDPAFRANTNNPDWDFNPNKDTWPDANKVGAGAFGLGFTPPHGGLLGWSPQAQGIMQTLPANPPPASTNRQSGRQPTPLLPKVLHKRTLMPLSYQHFRKLLLLDNEAGPLEEELPRLADEDLNRRVAEDLNLGNLNVSIPWTHKVGNFTGLYSSSVPVFNPHWKTPSFPNIHLHQDIIKKCEQFVGPLTVNEKRRLKLIMPARFYPNFTKYLPLDKGIKPYYPEHLVNHYFHTRHYLHTLWKAGILYKRVSTHSASFCGSPYSWEQELQHGAESFHQQSSGILSRPSVGSSLQSKHQQSRLGLQSQQGHLARRQQGRSWSIRTRVHPTARRPSGVEPSGSGHNANLASKSASCLYQSTVRTAAYPAVSTSENHSSSGHAVELHNLPPNSARSQSERPVSPCWWLQFRNSKPCSDYCLSHIVNLLEDWGPCHKRTLGLSAMSPPLRTTHPQAMQWNSTTFHQTLQDPRVRGLYLPAGGSSSGTVNPVPTTASPTLSTSSRIGDPALNQFLPKVLHKRSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAITVNAHQFLPKQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYILSAMSTTDL

SEQ ID NO: 108 - protein, 532 aa, synthetic construct
  REGION 1..532: note = HBV design 3
MQTDRTGETALHLAARYSRSDFLPSDFFPSVADAGVWIRTPPADNMEYLVSFGVWPLHAAVSADCWGELMTLRNRATDLGGPNLDNILMHDILRSFIPLLPLILAARLAVSTLPETTVVRRSHADVFLGGPPVCLDDLFLLTRILTIALHWAAAVNNVLTFGRETVLEYGANKWLSLLVPFVNNRFLKQQYMNLPLFLAAREGSYEDLLDTASALYANRFLSKQYMDLDHMTVSTKLCKIPRDLWFHISCLTFIVRLLDLEVSQTSKLTRQTDRTGETALHLAARYSRSDLTTVPAASLLAADAGLSRYVARLDNMKLHLYSHPIPLHAAVSADGLSPTVWLSVRNRATDLFLLSLGIHLMHDSLYADSPSVPLILAARLAVHKRTLGLSAMSHADVTLCIPHVAVDDLLLLKATLCIALHWAAAVNNVQFLPKVLHKRGANKALMPLYACINNRTVNAHQFLPKPLFLAAREGSYELPKVLHKRTLANRVLHKRTLGLDHMLSAMSTTDLPRDLLVPFVQWFVIVRLLDLE

SEQ ID NO: 109 - protein, 368 aa, synthetic construct
  REGION 1..368: note = HBV design 4
KKFLPSDFFPSVKKCWGELMTLKKGVWIRTPPAKKSTLPETTVVRRKKLTFGRETVLEYKKDLLDTASALYKKLWFHISCLTFKKEYLVSFGVWKKGGPNLDNILKKLTTVPAASLLAKKILRSFIPLLKKFLGGPPVCLKKFLLTRILTIKKWLSLLVPFVKKGLSPTVWLSVKKLLVPFVQWFVKKFLKQQYMNLKKFLSKQYMDLKKTVSTKLCKIKKGLSRYVARLKKKLHLYSHPIKKFLLSLGIHLKKSLYADSPSVKKALMPLYACIKKLLLKATLCIKKTLCIPHVAVKKVLHKRTLGLKKLPKVLHKRTLKKHKRTLGLSAMKKQFLPKVLHKRKKTVNAHQFLPKKKLSAMSTTDLKK

SEQ ID NO: 110 - protein, 20 aa, synthetic construct
  REGION 1..20: note = splice primer
TGCCAAGAGTGACGTGTCCA

SEQ ID NO: 111 - protein, 9 aa, synthetic construct
  REGION 1..9: note = Cleavable linker
VSQTSKLTR

SEQ ID NO: 112 - protein, 9 aa, synthetic construct
  REGION 1..9: note = HBV Peptides from Surface 1
GGPNLDNIL

SEQ ID NO: 113 - protein, 11 aa, synthetic construct
  REGION 1..11: note = HBV Peptides from Surface 2
LTTVPAASLLA

SEQ ID NO: 114 - protein, 9 aa, synthetic construct
  REGION 1..9: note = HBV Peptides from Surface 3
ILRSFIPLL

SEQ ID NO: 115 - protein, 9 aa, synthetic construct
  REGION 1..9: note = HBV Peptides from Surface 4
FLGGPPVCL

SEQ ID NO: 116 - protein, 9 aa, synthetic construct
  REGION 1..9: note = HBV Peptides from Surface 5
FLLTRILTI

SEQ ID NO: 117 - protein, 9 aa, synthetic construct
  REGION 1..9: note = HBV Peptides from Surface 6
WLSLLVPFV

SEQ ID NO: 118 - protein, 10 aa, synthetic construct
  REGION 1..10: note = HBV Peptides from Surface 7
GLSPTVWLSV

SEQ ID NO: 119 - protein, 10 aa, synthetic construct
  REGION 1..10: note = HBV Peptides from Surface 8
LLVPFVQWFV

SEQ ID NO: 120 - protein, 9 aa, synthetic construct
  REGION 1..9: note = Hepatitis B Spliced Protein 1
LLLKATLCI

SEQ ID NO: 121 - protein, 9 aa, synthetic construct
  REGION 1..9: note = Hepatitis B Spliced Protein 2
TLCIPHVAV

SEQ ID NO: 122 - protein, 9 aa, synthetic construct
  REGION 1..9: note = HBx protein
VLHKRTLGL

SEQ ID NO: 123 - protein, 10 aa, synthetic construct
  REGION 1..10: note = HBx protein
LPKVLHKRTL

SEQ ID NO: 124 - protein, 10 aa, synthetic construct
  REGION 1..10: note = HBx protein
HKRTLGLSAM

SEQ ID NO: 125 - protein, 10 aa, synthetic construct
  REGION 1..10: note = HBx protein
QFLPKVLHKR

SEQ ID NO: 126 - protein, 10 aa, synthetic construct
  REGION 1..10: note = HBx protein
TVNAHQFLPK

SEQ ID NO: 127 - protein, 9 aa, synthetic construct
  REGION 1..9: note = HBx protein
LSAMSTTDL

SEQ ID NO: 128 - protein, 21 aa, synthetic construct
  REGION 1..21: note = splice probe
CCCAGGTCCAACTGCAGCCGG

SEQ ID NO: 129 - protein, 10 aa, synthetic construct
  REGION 1..10: note = Rigid Linker
EAAAKEAAAK

SEQ ID NO: 130 - DNA, 12 bp, synthetic construct
  misc_feature 1..12: note = Furin Linker
  source 1..12: mol_type = other DNA
agagctaagagg

SEQ ID NO: 131 - protein, 5 aa, synthetic construct
EAAAK

SEQ ID NO: 132 - protein, 212 aa, Synthetic construct
MQLFHLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 133 - protein, 214 aa, Synthetic construct
LQLFHLCLIIFCTCSTFQASKLCLGWLWDMDIDTYKEFGATAELLSFLPADFFPSVRDLLDTAAALYRDALESPEHCTPNHTAIRQAVVCWVDLMTLASWVGNNLQDQIARDLIVNYVNTNVGLKFRQILWFHISCLTFGRDTVIEYLVSFGVWIRTPTPYRPQNAPILSTLPENCVIRQRDRCRTPRRRTPSPRRRRSQSPRRRRSKSPAPQC

SEQ ID NO: 134 - protein, 212 aa, Synthetic construct
MQLFHLCLIISCSCPTVQASKLCLGWLWGMDIDPYKEFGASVELLSFLPTDFFPSIRDLLDTATALYREALESPEHCSPHHTALRQAILCWGELMNLATWVGVNLDDPTSRELVVGYVNVNMGLKLRQLLWFHISCLTFGRETVLEYLVSFGVWIRTPQAYRPPNAPILSTLPETTVVRCRGRSPRRRTPSPRRRRSQSPRRRRSQSRGSQC

SEQ ID NO: 135 - protein, 212 aa, Synthetic construct
MQLFHLCLIISCSCPTVQASKLCLGWLWGMDIDPYKEFGASVELLSFLPSDFFPSIRDLLDTASALYREALESPEHCSPHHTALRQAILCWGELMNLATWVGGNLEDPASRELVVSYVNVNMGLKIRQLLWFHISCLTFGRETVLEYLVSFGVWIRTPLAYRPPNAPILSTLPETTVVRRRGRSPRRRTPSPRRRRSQSPRRRRSQSRESQC

SEQ ID NO: 136 - protein, 212 aa, Synthetic construct
MQLFHLCLIISCSCPTVQASKLCLGWLWGMDIDPYKEFGASVELLSFLPSDFFPSIRDLLDTASALYREALESPEHCSPHHTALRQAILCWGELMNLATWVGTNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 137 - protein, 15 aa, Synthetic construct
HQLDPAFGANSTNPD

SEQ ID NO: 138 - protein, 23 aa, Synthetic construct
NSTTFHQALLDPRVRGLYFPAGG

SEQ ID NO: 139 - protein, 14 aa, Synthetic construct
ILPKVLHKRTLGLS

SEQ ID NO: 140 - protein, 26 aa, Synthetic construct
EYIKDCVFKDWEELGEEIRLKVFVLG

SEQ ID NO: 141 - protein, 9 aa, Synthetic construct
VCWGELMNL

SEQ ID NO: 142 - protein, 9 aa, Synthetic construct
STLPETTVV

SEQ ID NO: 143 - protein, 360 aa, Synthetic construct
MDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVMPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRL

SEQ ID NO: 144 - protein, 148 aa, Synthetic construct
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTV

SEQ ID NO: 145 - protein, 1051 aa, Synthetic construct
MDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVMPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHVLQAGFFLLTRILTIPQSLDSWWTSLSFLGGTTVCLGQWGLSPFLLAQFTSAICSVVRRAFPHCLAFSFPDHQLDPAFRANTANPDWDFNPNKDTWPDANKVGAGAGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSWASARFSWLSLLVPFVQWFVGLSPTVWLSVYKAFLCKQYLNLYPVARQRPGLCQVFAHATPTGWGLVMGHQRMRGTFLENITSGFLGPLLVLQAGFFLLTRILTIPQSSRKYTSFPWLLGCAANWILRGTSFVYYPSALNPYHDPSRGRLGLSRPLLRLPFRPTTGRTSLYADSPSVPSHLPDRVHFASPLHVAWRPP

SEQ ID NO: 146 - protein, 903 aa, synthetic construct
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHVLQAGFFLLTRILTIPQSLDSWWTSLSFLGGTTVCLGQWGLSPFLLAQFTSAICSVVRRAFPHCLAFSFPDHQLDPAFRANTANPDWDFNPNKDTWPDANKVGAGAGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSWASARFSWLSLLVPFVQWFVGLSPTVWLSVYKAFLCKQYLNLYPVARQRPGLCQVFADATPTGWGLVMGHQRMRGTFLENITSGFLGPLLVLQAGFFLLTRILTIPQSSRKYTSFPWLLGCAANWILRGTSFVYVPSALNPTDDPSRGRLGLSRPLLRLPFRPTTGRTSLYADSPSVPSHLPDRVHFASPLHVAWRPP

SEQ ID NO: 147 - protein, 1014 aa, synthetic construct; identical to SEQ ID NO: 106

SEQ ID NO: 148 - protein, 1054 aa, Synthetic construct
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYIMQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQMAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSAGPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSYMDDVVLGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSAPLPIHTAELLAACFARSRSGANILGTDNSVVLSRKYTSFPWLLGCAANWILRGTSFVYVPSALNPADDPSRGRLGPCRPLLHLPFRPTTGRTSLYADSPSVPSHLPDRVHFASPLHVAWRPP

(19) United States
(12) Patent Application Publication
(10) Pub. No.: US 2025/0223320 A1
(43) Pub. Date: Jul. 10, 2025

(54) HEPATITIS B VACCINES AND USES OF THE SAME

(71) Applicant: PRECIGEN, INC., Germantown, MD (US)

(72) Inventors: Douglas E. BROUGH, Germantown, MD (US); Cheryl G. BOLINGER, Germantown, MD (US); Ramya YARLAGADDA, Germantown, MD (US); Vinodhbabu KURELLA, Germantown, MD (US); Prabakaran PONRAJ, Germantown, MD (US); Simon METENOU, Germantown, MD (US); Kuan-Fu DING, Germantown, MD (US)

(73) Assignee: PRECIGEN, INC., Germantown, MD (US)

(21) Appl. No.: 19/023,657

(22) Filed: Jan. 16, 2025

Related U.S. Application Data
(63) Continuation of application No. 18/074,302, filed on Dec. 2, 2022, now Pat. No. 12,234,261.
Application No. 18/074,302 is a continuation of application No. 16/978,570, filed on Sep. 4, 2020, now Pat. No. 11,608,362.
(60) Provisional application No. 62/639,354, filed on Mar. 6, 2018.

(51) Int. Cl. C07K 14/005; A61K 39/12; A61K 39/29; A61K 48/00; C07K 14/02; C12N 7/00; C12N 15/861

(52) U.S. Cl. CPC C07K 14/005; A61K 39/12; A61K 39/292; A61K 48/0066; C07K 14/02; C12N 7/00; C12N 15/861; C07K 2319/33; C12N 2710/20022; C12N 2710/20034

(57) ABSTRACT

Provided herein are engineered hepatitis B virus (HBV) molecular vaccine constructs. Vaccine constructs can also include ligand-inducible engineered gene switch systems for modulating expression of heterologous genes, such as a cytokines, in host cells.

### REFERENCE TO SEQUENCE LISTING

The instant application contains a Sequence Listing that has been submitted electronically in XML format and is hereby incorporated by reference in its entirety. The XML copy was created on Jan. 10, 2025, is named 18074302CONSeqList.xml and is 403,425 bytes in size.

### FIELD OF THE DISCLOSURE

The present disclosure relates to improved, broad-spectrum HBV molecular vaccines designed via use of advanced principles in bioinformatics and protein engineering.

### BACKGROUND OF THE DISCLOSURE

Chronic hepatitis B is a disease involving multiple viral (HepB or HBV) genotypes. HBV genotypes/subgenotypes have been increasingly associated with differences in clinical and virological characteristics, such as severity of liver disease and response to antiviral therapies. Infection with HBV causes hepatitis that can result in cirrhosis, liver failure and hepatocellular carcinoma (HCC). The diagnosis of HBV is based on the serological findings. There is no cure for chronic HBV infection. Currently available treatment options are aimed at slowing the progression of cirrhosis and viral replication, reducing the incidence of HCC and liver failures.

The present disclosure relates to improved, broad-spectrum HBV molecular vaccines designed via use of advanced principles in bioinformatics and protein engineering. These novel HBV vaccines can be used as a therapeutic vaccine against HBV related diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

Provided herein is a non-naturally occurring polynucleotide encoding a polypeptide comprising at least one or more immune response-inducing hepatitis B virus (HBV) polypeptides.

In some embodiments, said non-naturally occurring polynucleotide encodes a polypeptide comprising said one or more HBV polypeptides. In some embodiments, said one or more HBV polypeptides comprises an HBV Core peptide. In some embodiments, said HBV Core peptide has any one of Core peptide sequences as shown in Table 3. In some embodiments, said one or more HBV peptides comprises an HBV Surface peptide. In some embodiments, said HBV Surface peptide has any one of Surface peptide sequences as shown in Table 3. In some embodiments, said one or more HBV peptides comprises an HBV Pol peptide. In some embodiments, said HBV Pol peptide has any one of Pol peptide sequences as shown in Table 3. In some embodiments, said one or more HBV peptides comprises an HBV HBSP/HBx peptide. In some embodiments, said HBV HBSP/HBx peptide has any one of HBSP/HBx peptide sequences as shown in Table 3. In some embodiments, said one or more HBV peptides comprises a KK linker. In some embodiments, said KK linker connects any one of peptide sequences as shown in Table 3 to any other peptide sequences as shown in Table 3.

Provided herein is a polynucleotide comprising any of the polynucleotides provided herein, further comprising one or more polynucleotides encoding a gene switch system for inducible control of heterologous gene expression, wherein heterologous gene expression is regulated by said gene switch system; and, wherein said heterologous gene comprises any of the polynucleotide described herein. In some embodiments, said gene switch system is an ecdysone receptor-based (EcR-based) gene switch system. In some embodiments, said one or more HBV polypeptides is for use in a vaccine.

Provided herein is a vector comprising any of the polynucleotides provided herein. In some embodiments, said vector is an adenoviral vector. In some embodiments, said adenoviral vector is a gorilla adenoviral vector.

Provided herein is a method of regulating the expression of a heterologous gene in a cell, the method comprising: introducing into said cell one or more polynucleotides that comprise (i) an repressible or inducible gene switch, and (ii) a heterologous immune response-inducing gene, wherein expression of said heterologous immune response-inducing gene is regulated by said gene switch, wherein said heterologous immune response-inducing gene encodes at least one of one or more HBV polypeptides; and exposing said cell to a compound in an amount sufficient to repress or induce expression of said heterologous immune response-inducing gene.

In some embodiments, said target cell is a mammalian cell in a method of regulating the expression of a heterologous gene in a cell described herein. In some embodiments, said gene switch comprises a ligand binding domain derived from at least one of an ecdysone receptor (EcR), a ubiquitous receptor, an orphan receptor 1, an NER-1, a steroid hormone nuclear receptor 1, a retinoid X receptor interacting protein-15, a liver X receptor 3, a steroid hormone receptor like protein, a liver X receptor, a liver X receptor α, a farnesoid X receptor, a receptor interacting protein 14, and a famesol receptor.

Provided herein is a polynucleotide encoding any of the presently described polypeptide constructs. Also provided herein is a vector comprising said polynucleotide. In some embodiments, said vector is an adenoviral vector. In some embodiments, said adenoviral vector is a gorilla adenoviral vector.

Provided herein is a vector, wherein said vector comprises a polynucleotide that encodes at least one HBV peptide, wherein said vector is an adenoviral vector.

Provided herein is a vector, wherein said vector comprises a polynucleotide that encodes at least one HBV peptide, wherein said vector is an adenoviral vector, wherein said adenoviral vector is a gorilla adenoviral vector.

Provided herein is a polypeptide construct, wherein said polypeptide construct comprises an HBV HBx domain and at least one of an HBV Pol domain, an HBV Core domain, an HBV pre-Core domain or an HBV Surface domain. Also provided herein is a polypeptide construct, wherein said polypeptide construct comprises a pre-Core domain and at least one of an HBV Pol domain, an HBV HBx domain or an HBV Surface domain. In some embodiments, said HBV HBx domain has a sequence as shown in SEQ ID NO: 98. In some embodiments, said HBV Pol domain comprises a deletion of at least one amino acid as compared to a wildtype HBV Pol domain. In some embodiments, said deletion comprises a deleted portion of said wildtype HBV Pol domain, wherein said deleted portion comprises at least one of amino acids 538-544 or amino acids 710-742. In some embodiments, said deleted portion comprises both of amino acids 538-544 and amino acids 710-742. In some embodiments, said HBV Pol domain has a sequence as shown in SEQ ID NO: 99. In some embodiments, said HBV Surface domain comprises at least one of a PreS1 domain, a PreS2 domain and an S domain. In some embodiments, said HBV Surface domain comprises an HBV S domain. In some embodiments, said Surface domain has a sequence as shown in SEQ ID NO: 100. In some embodiments, said polypeptide construct further comprises an HBV Core domain. In some embodiments, said polypeptide construct comprises a Core portion, wherein said Core portion comprises said HBV Core domain and said HBV pre-Core domain. In some embodiments, said Core portion has a sequence as shown in SEQ ID NO: 101. In some embodiments, said polypeptide construct comprises each of SHB (Env), HBeAg, HBx, and Pol domains. In some embodiments, said polypeptide construct comprises a structure, from N-terminus to C-terminus, of said SHB (Env), HBeAg, HBx, and Pol domains. In some embodiments, said SHB (Env) domain has a sequence as shown in SEQ ID NO: 102. In some embodiments, said HBeAg domain has a sequence as shown in SEQ ID NO: 103. In some embodiments, said HBx domain has a sequence as shown in SEQ ID NO: 104. In some embodiments, said Pol domain has a sequence as shown in SEQ ID NO: 105. In some embodiments, said polypeptide construct has a sequence as shown in SEQ ID NO: 106.

Provided herein is a polypeptide construct, wherein said polypeptide construct comprises one or more HBV HBx linkers and at least one of a Core domain, a Surface domain and a Pol domain, wherein one of said Core domain, said Surface domain and said Pol domain is connected to another of said Core domain, said Surface domain and said Pol domain by said one or more HBx linkers. In some embodiments, said Surface domain comprises at least one of an HBV PreS1 domain, an HBV PreS2 domain and an HBV S domain. In some embodiments, said one or more HBV HBx linkers comprises multiple HBV HBx linkers. In some embodiments, at least two of said multiple HBV HBx linkers differ in an amino acid sequence. In some embodiments, said HBV HBx linker has a sequence as shown in any one of HBx-1, HBx-2, HBx-3, HBx-4, HBx-5 or HBx-6 of Table 3. In some embodiments, said Core domain is adjacent to said Surface domain. In some embodiments, said Surface domain comprises a PreS1 domain. In some embodiments, said Surface domain is connected to said Core domain by one of said one or more HBx linkers. In some embodiments, said Pol domain is adjacent to a Surface domain. In some embodiments, said Surface domain comprises at least one of a PreS1 domain, a PreS2 domain and an S domain. In some embodiments, said Surface domain comprises said PreS1 domain, and an N-terminal portion of said Pol domain is adjacent to said PreS1 domain. In some embodiments, said N-terminal portion of said Pol domain is connected to said PreS1 domain by one of said one or more HBx linkers. In some embodiments, said Surface domain comprises said PreS2 domain, and an N-terminal portion of said Pol domain is adjacent to said PreS2 domain. In some embodiments, said N-terminal portion of said Pol domain is connected to said PreS2 domain by one of said one or more HBx linkers. In some embodiments, said Surface domain comprises said PreS2 domain, and a C-terminal portion of said Pol domain is adjacent to said PreS2 domain. In some embodiments, said C-terminal portion of said Pol domain is connected to said PreS2 domain by one of said one or more HBx linkers. In some embodiments, said Surface domain comprises said S domain, and a C-terminal portion of said Pol domain is adjacent to said S domain. In some embodiments, said C-terminal portion of said Pol domain is connected to said S domain by one of said one or more HBx linkers. In some embodiments, said polypeptide construct has a sequence as shown in SEQ ID NO: 107.

Provided herein is a polypeptide construct comprising an ankyrin-like repeat domain and one or more HBV peptides. In some embodiments, said ankyrin-like repeat protein is a human ankyrin-like repeat protein. In some embodiments, said one or more HBV peptides has a sequence as shown in any one of the amino acid sequences of Table 3. In some embodiments, said one or more HBV peptides comprises one or more of a Core peptide, a Surface peptide, a Pol peptide and an HBSP/HBx peptide. In some embodiments, said one or more HBV peptides comprises a Core peptide, and said Core peptide has a sequence as shown in any one of the Core amino acid sequences of Table 3. In some embodiments, said one or more HBV peptides comprises a Surface peptide, and said Surface peptide has a sequence as shown in any one of the Surface amino acid sequences of Table 3. In some embodiments, said one or more HBV peptides comprises a Pol peptide, and said Pol peptide has a sequence as shown in any one of the Pol amino acid sequences of Table 3. In some embodiments, said one or more HBV peptides comprises an HBSP/HBx peptide, and said HBSP/HBx peptide has a sequence as shown in any one of the HBSP/HBx amino acid sequences of Table 3. In some embodiments, said polypeptide construct has a sequence as shown in SEQ ID NO: 108.

Provided herein is a polypeptide construct, wherein said polypeptide construct comprises at least two HBV amino acid sequences as shown in Table 3, wherein said at least two HBV amino acid sequences are connected by a peptide linker, wherein said peptide linker is a KK linker. In some embodiments, said comprises at least two HBV amino acid sequences comprise at least one of a Core peptide, a Surface peptide, a Pol peptide and an HBSP/HBx peptide as shown in Table 3. In some embodiments, said at least two HBV amino acid sequences comprise each of the amino acid sequences as shown in Table 3. In some embodiments, said each of the amino acid sequences is connected to another of said each of the amino acid sequences by said KK linker. In some embodiments, said polypeptide construct has a sequence as shown in SEQ ID NO: 109. In some embodiments, any of the polypeptide constructs described herein is for use in a vaccine. Also provided herein is a polynucleotide encoding any of the polypeptide constructs presently described. Also provided herein is a vector comprising said polynucleotide. In some embodiments, said vector is an adenoviral vector. In some embodiments, said adenoviral vector is a gorilla adenoviral vector.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic of chronic HBV infection.

FIG. 2 is a schematic showing history of chronic HBV infection.

FIG. 3A is a schematic overview of hepatitis B virus. HBV DNA is 3.2 kb circular, enveloped, partially double strand DNA genome. HBV has four genes (S, C, P, and X). The S gene codes for envelope (lipid bilayer) surface protein (HBsAg) consisting of small surface protein (S), medium surface protein (S+PreS2), and large surface protein (S+PreS2+PreS1). The C gene codes for the capsid or core proteins. The C gene has a precore and a core region. If translation is initiated at the precore region, the protein is HBeAg. If translation is initiated at the core region, the protein is HBcAg. The P gene codes for the DNA polymerase (Pol). The X gene codes for the x protein (HBxAg). FIG. 3B is an overview of hepatitis B virus genome. The HBV genome comprises Pol (832 amino acids) comprising TP, SP, RT and RH; PreS1 (108 amino acids); PreS2 (55 amino acids); S (226 amino acids); PreC (29 amino acids); C (183 amino acids); and HBx (154 amino acids). FIG. 3C shows a schematic of the HBV genome encoding several overlapping viral proteins, including the polymerase core, core envelop (Pre-S1, S2, S), HBe, and HBx proteins. FIG. 3D shows a schematic of the HBV genome encoding several overlapping viral proteins, including the polymerase core, core envelop (Pre-S1, S2, S), HBe, and HBx proteins.

FIG. 4 shows a schematic overview of HBV infection mechanism.

FIG. 5 is schematic overall workflow implemented for designing HBV vaccine antigens.

FIG. 6 shows schematic structural diagrams of HBV designs 1-5. HBV designs 1 and 2 were designed based on clade D consensus. For HBV design 3, human ankyrin-like repeat (ALR) protein scaffold (PDB code 1QYM) peptides were grafted at the helical and loop regions in a tandem manner. Two ALR scaffolds were used, connected by a cleavable linker (VSQTSKLTR) (SEQ ID NO: 111). HBV design 4 epitopes were separated by KK linkers. Different linkers, such as EAAAK linkers (SEQ ID NO: 131) were used in HBV design 5 to connect the peptides.

FIG. 7 shows an overview of IL-12 promoting immune response by activating NK cells and T cells.

FIG. 8 shows various structural components of diverse IL-12 ligand-inducible gene switch vector systems.

FIG. 9A shows NetMHC4.0 antigenicity predictions for HBe. Predicted strong and weak binding peptides indices were plated against peptide locations. FIG. 9B shows NetMHC4.0 antigenicity predictions for HBe. First and second order differentials were employed on density plots in order to identify peaks. FIG. 9C shows amino acid sequences aligned against consensus sequences in order to determine coverage across HBV subtypes (SEQ ID NOs: 132-136).

FIG. 10 is a schematic representation of TG1050 and HBV design 1 highlighting the fused domains of different HBV protein. Homology models were used to further assess the design.

FIG. 11 is a schematic representation of the HBV design 2 consisting of all three major proteins (core, surface splice variants, and polymerase) linked with HBx peptides (HBx-1 (SEQ ID NO: 122), HBx-2 (SEQ ID NO: 123), HBx-3 (SEQ ID NO: 124), HBx-4 (SEQ ID NO: 125), HBx-5 (SEQ ID NO: 126), and HBx-6 (SEQ ID NO: 127).

FIG. 12A shows a structure of core HBV protein domain. FIG. 12B shows a structure of HBV preS1 (SEQ ID NO: 137) and S2 peptides (SEQ ID NO: 138). FIG. 12C shows structures of HBV HBx (SEQ ID NOs: 139 and 140). FIG. 12D shows structures of four core peptides-MHC complexes (SEQ ID NOs: 141 and 142).

FIG. 13 shows a homology model of HBV design 4 based on multi-epitope antigens.

FIG. 14 is a schematic illustration showing short and long primer and probe sets generated for RNA qPCR relative expression assay. Specific primers were designed for each HBV antigen design.

FIG. 15A shows core sequence comparisons of HBV design 1 and TG1050 control (SEQ ID NOs: 143 and 144). FIG. 15B shows sequence comparisons of HBV design 1 and TG1050 control (SEQ ID NOs: 145 and 146). FIG. 15C shows sequence comparisons of HBV design 1 and TG1050 control (SEQ ID NOs: 147 and 148).

FIG. 16 is a schematic representation of HBV design 5.

### DETAILED DESCRIPTION OF THE DISCLOSURE

The following description and examples illustrate embodiments of the present disclosure in detail.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “isolated” and its grammatical equivalents as used herein refer to the removal of a nucleic acid from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Polynucleotide”, “oligonucleotide”, “polynucleotide construct”, “gene”, “gene construct”, “heterologous gene” and their grammatical equivalents as used herein refer to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). Phage or viral vectors can be introduced into host cells, after growth of infectious particles in suitable packaging cells, many of which are commercially available.

“Polypeptide”, “peptide” “polypeptide construct” and “peptide construct” and their grammatical equivalents as used herein refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and α-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The term “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. Vector also can comprise a selectable marker gene.

The term “adenovirus,” as used herein, refers to an adenovirus that retains the ability to participate in the adenovirus life cycle and has not been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5^{th} ed., Knipe et al. (eds.), Lippincott Williams & Wilkins, Philadelphia, PA (2006)). The term “adenoviral vector,” as used herein, refers to an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

The term “selectable marker gene” as used herein refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150:1 (1981); Santerre et al., Gene, 30: 147 (1984); Kent et al., Science, 237: 901-903 (1987); Wigler et al., Cell, 11: 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

The term “coding sequence” as used herein refers to a segment of a polynucleotide that codes for protein. The region or sequence is bounded nearer the 5′ end by a start codon and nearer the 3′ end with a stop codon. Coding sequences can also be referred to as open reading frames.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The terms “induce”, “induction” and its grammatical equivalents as used herein refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch. The inducible promoter can be a gene switch ligand inducible promoter. In some cases, an inducible promoter can be a small molecule ligand-inducible two polypeptide ecdysone receptor-based gene switch. In some cases, a gene switch can be selected from ecdysone-based receptor components as described in, but without limitation to, any of the systems described in: International Patent Applications WO 2001/070816; WO 2002/029075; WO 2002/066613; WO 2002/066614; WO 2002/066612; WO 2002/066615; WO 2003/027266; WO 2003/027289; WO 2005/108617; WO 2009/045370; WO 2009/048560; WO 2010/042189; WO 2010/042189; WO 2011/119773; and WO 2012/122025; and U.S. Pat. Nos. 7,091,038; 7,776,587; 7,807,417; 8,202,718; 8,105,825; 8,168,426; 7,531,326; 8,236,556; 8,598,409; 8,715,959; 7,601,508; 7,829,676; 7,919,269; 8,030,067; 7,563,879; 8,021,878; 8,497,093; 7,935,510; 8,076,454; 9,402,919; 9,493,540; 9,249,207; and 9,492,482, each of which is incorporated by reference in its entirety).

The term “gene switch” or “genetic switch” refers to the combination of a response element associated with a promoter, and for instance, an EcR based system, which, in the presence of one or more ligands, modulates the expression of a gene into which the response element and promoter are incorporated. Tightly regulated inducible gene expression systems or gene switches are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

“Sleeping Beauty (SB) Transposon System” refers a synthetic DNA transposon system for to introducing DNA sequences into the chromosomes of vertebrates. Some exemplary embodiments of the system are described, for example, in U.S. Pat. Nos. 6,489,458, 8,227,432, 9,228,180 and WO/2016/145146. The Sleeping Beauty transposon system is composed of a Sleeping Beauty (SB) transposase and a SB transposon. In embodiments, the Sleeping Beauty transposon system can include the SB11 transposon system, the SB100X transposon system, or the SB110 transposon system.

“Transposon” or “transposable element” (TE) is a vector DNA sequence that can change its position within the genome, sometimes creating or reversing mutations and altering the cell's genome size. Transposition often results in duplication of the TE. Class I TEs are copied in two stages: first they are transcribed from DNA to RNA, and the RNA produced is then reverse transcribed to DNA. This copied DNA is then inserted at a new position into the genome. The reverse transcription step is catalyzed by a reverse transcriptase, which can be encoded by the TE itself. The characteristics of retrotransposons are similar to retroviruses, such as HIV. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons can be identified by short direct repeats which can be created by the staggered cut in the target DNA and filling in by DNA polymerase, followed by a series of inverted repeats important for the TE excision by transposase. Cut-and-paste TEs can be duplicated if their transposition takes place during S phase of the cell cycle when a donor site has already been replicated, but a target site has not yet been replicated. Transposition can be classified as either autonomous or non-autonomous in both Class I and Class II TEs. Autonomous TEs can move by themselves while non-autonomous TEs require the presence of another TE to move. This is often because non-autonomous TEs lack transposase (for class II) or reverse transcriptase (for class I).

“Transposase” refers an enzyme that binds to the end of a transposon and catalyzes the movement of the transposon to another part of the genome by a cut and paste mechanism or a replicative transposition mechanism.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

“T helper cells” (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}9, T_{H}17, T_{H}22 or T_{FH} (T follicular helper cells), which secrete different cytokines to facilitate different types of immune responses. Signaling from the APCs directs T cells into particular subtypes.

“Cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

“Memory T cells” are a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (T_{CM} cells) and two types of effector memory T cells (T_{EM} cells and T_{EMRA} cells). Memory cells can be either CD4+ or CD8+. Memory T cells typically express the cell surface proteins CD45RO, CD45RA and/or CCR7.

“Regulatory T cells” (Treg cells), formerly known as suppressor T cells, play a role in the maintenance of immunological tolerance. Their major role is to shut down T cell-mediated immunity toward the end of an immune reaction and to suppress autoreactive T cells that escaped the process of negative selection in the thymus.

“Natural killer T cells” (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both T helper (T_{H}) and cytotoxic T (TC) cells (i.e., cytokine production and release of cytolytic/cell killing molecules). They are also able to recognize and eliminate some tumor cells and cells infected with herpes viruses.

“Adoptive T cell transfer” refers to the isolation and ex vivo expansion of tumor specific T cells to achieve greater number of T cells than what could be obtained by vaccination alone or the patient's natural tumor response. The tumor specific T cells are then infused into patients with cancer in an attempt to give their immune system the ability to overwhelm remaining tumor via T cells which can attack and kill cancer. There are many forms of adoptive T cell therapy being used for cancer treatment; culturing tumor infiltrating lymphocytes or TIL, isolating and expanding one particular T cell or clone, and even using T cells that have been engineered to potently recognize and attack tumors.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” can be for example proteins that are members of the Ig-superfamily which are able to selectively bind a partner. MHC molecules and T cell receptors are such molecules. In one embodiment, the antibody-like molecule is an TCR. In one embodiment, the TCR has been modified to increase its MHC binding affinity.

The terms “fragment of an antibody,” “antibody fragment,” “functional fragment of an antibody,” “antigen-binding portion” or its grammatical equivalents are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Non-limiting examples of antibody fragments include (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Pat. No. 8,603,950.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Alternatively or additionally, the functional variants can comprise the amino acid sequence of the reference protein with at least one non-conservative amino acid substitution.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein.

The term “ankyrin” refers to a family of adaptor proteins that mediate the attachment of integral membrane proteins to the spectrin-actin based membrane cytoskeleton. Ankyrins have binding sites for the beta subunit of spectrin and at least 12 families of integral membrane proteins. This linkage is required to maintain the integrity of the plasma membranes and to anchor specific ion channels, ion exchangers and ion transporters in the plasma membrane. Ankyrins contain four functional domains: an N-terminal domain that contains 24 tandem ankyrin repeats, a central domain that binds to spectrin, a death domain that binds to proteins involved in apoptosis, and a C-terminal regulatory domain that is highly variable between different ankyrin proteins. The 24 tandem ankyrin repeats are responsible for the recognition of a wide range of membrane proteins. These 24 repeats contain 3 structurally distinct binding sites ranging from repeat 1-14. These binding sites are quasi-independent of each other and can be used in combination. The interactions the sites use to bind to membrane proteins are non-specific and consist of: hydrogen bonding, hydrophobic interactions and electrostatic interactions. These non-specific interactions gives ankyrin the property to recognize a large range of proteins as the sequence doesn't have to be conserved just the properties of the amino acids. The quasi-independence means that if a binding site is not used, it won't have a large effect on the overall binding. These two properties in combination give rise to large repertoire of proteins ankyrin can recognize. Ankyrins are encoded by three genes (ANK1, ANK2 and ANK3) in mammals. Each gene in turn produces multiple proteins through alternative splicing.

The term “proliferative disease” as referred to herein refers to a unifying concept in which excessive proliferation of cells and/or turnover of cellular matrix contributes significantly to the pathogenesis of the disease, including cancer.

“Patient” or “subject” as used herein refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to chronic hepatitis B infection.

“Administering” is referred to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise a target cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment”, “treating”, or its grammatical equivalents refers to obtaining a desired pharmacologic and/or physiologic effect. In embodiments, the effect is therapeutic, i.e., the effect partially or completely cures a disease and/or adverse symptom attributable to the disease. To this end, the inventive method comprises administering a therapeutically effective amount of the composition comprising the host cells expressing the inventive nucleic acid sequence, or a vector comprising the inventive nucleic acid sequences.

The term “therapeutically effective amount”, therapeutic amount”, “immunologically effective amount”, “anti-tumor effective amount”, “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease onset).

### HBV Molecular Vaccine

Hepatitis B (HepB) is a potentially life-threatening liver infection caused by the hepatitis B virus (HBV). HBV can cause chronic infection and puts people at high risk of death from cirrhosis and liver cancer. The evolution of HBV is strikingly highlighted by the geographical distribution of the genotypes. HBV genotypes/subgenotypes have been increasingly associated with differences in clinical and virological characteristics, such as severity of liver disease and response to antiviral therapies. When comparing sequences, HBV is classified into eight genotypes (A to H), each with a distinct geographic distribution. Researchers have correlated an association between the different HBV genotypes and the severity and outcome of HBV disease.

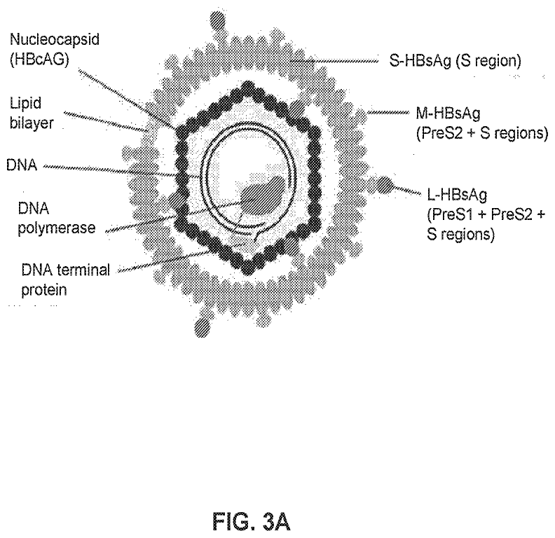

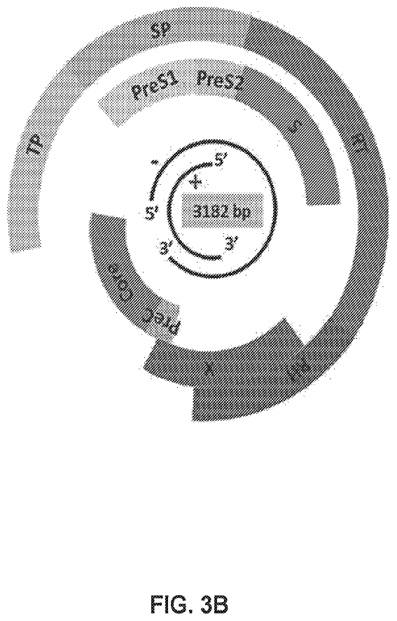

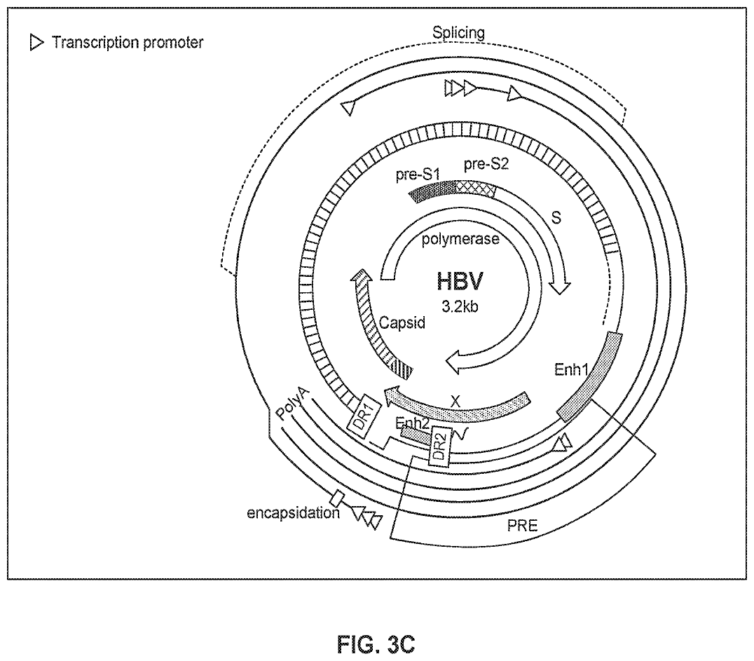

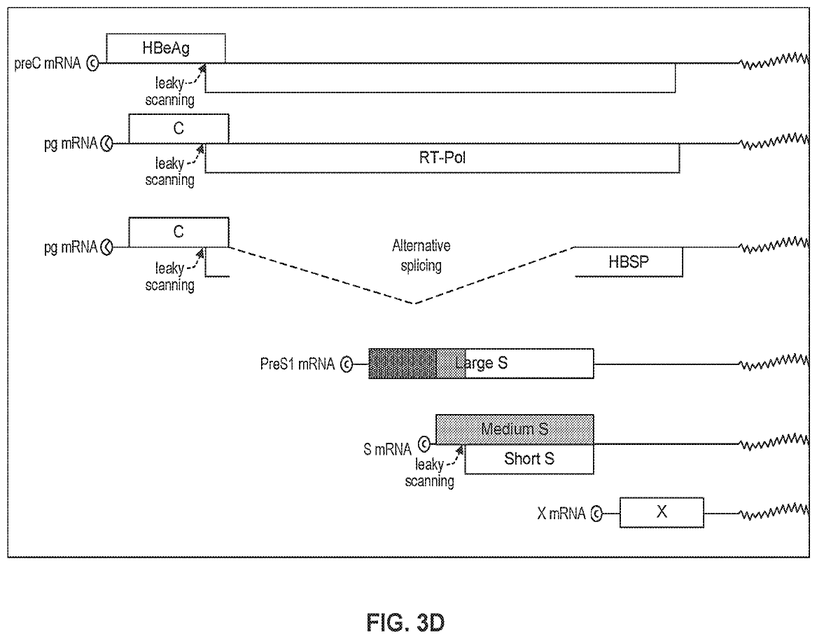

HBV is a double stranded DNA virus with high liver tropism. HBV DNA is 3.2 kb circular, enveloped, partially double strand DNA genome (FIGS. 3A-3D). HBV has four genes (S, C, P, and X). The S gene codes for envelope (lipid bilayer) surface protein (HBsAg) consisting of small surface protein (S), medium surface protein (S+PreS2), and large surface protein (S+PreS2+PreS1). The C gene codes for the capsid or core proteins. The C gene has a precore and a core region. If translation is initiated at the precore region, the protein is HBeAg. If translation is initiated at the core region, the protein is HBcAg. The P gene codes for the DNA polymerase (Pol). The X gene codes for the x protein (HBxAg) (FIGS. 3A-3D). The HBV genome comprises Pol (832 amino acids) comprising TP, SP, RT and RH; PreS1 (108 amino acids); PreS2 (55 amino acids); S (226 amino acids); PreC (29 amino acids); C (183 amino acids); and HBx (154 amino acids) (FIGS. 3A-3D).

Acute HBV infection is characterized by the presence of hepatitis B surface antigen (HBsAg) and immunoglobulin M (IgM) antibody to the core antigen, HBcAg. During the initial phase of infection, patients are also seropositive for hepatitis B e antigen (HBeAg). HBeAg is usually a marker of high levels of replication of the virus. The presence of HBeAg indicates that the blood and body fluids of the infected individual are highly infectious. Chronic infection is characterized by the persistence of HBsAg for at least 6 months (with or without concurrent HBeAg). Persistence of HBsAg is the principal marker of risk for developing chronic liver disease and liver cancer (hepatocellular carcinoma) later in life.

The hepatitis B virus can survive outside the body for at least 7 days. During this time, the virus can still cause infection if it enters the body of a person who is not protected by the vaccine. The incubation period of the hepatitis B virus is 75 days on average, but can vary from 30 to 180 days. The virus can be detected within 30 to 60 days after infection and can persist and develop into chronic hepatitis B. In highly endemic areas, hepatitis B is most commonly spread from mother to child at birth (perinatal transmission), or through horizontal transmission (exposure to infected blood), especially from an infected child to an uninfected child during the first 5 years of life. The development of chronic infection is very common in infants infected from their mothers or before the age of 5 years. Hepatitis B is also spread by percutaneous or mucosal exposure to infected blood and various body fluids, as well as through saliva, menstrual, vaginal, and seminal fluids. Sexual transmission of hepatitis B can also commonly occur. Infection in adulthood leads to chronic hepatitis in less than 5% of cases. Transmission of the virus can also occur through the reuse of needles and syringes either in health-care settings or among persons who inject drugs. In addition, infection can occur during medical, surgical and dental procedures, through tattooing, or through the use of razors and similar objects that are contaminated with infected blood.

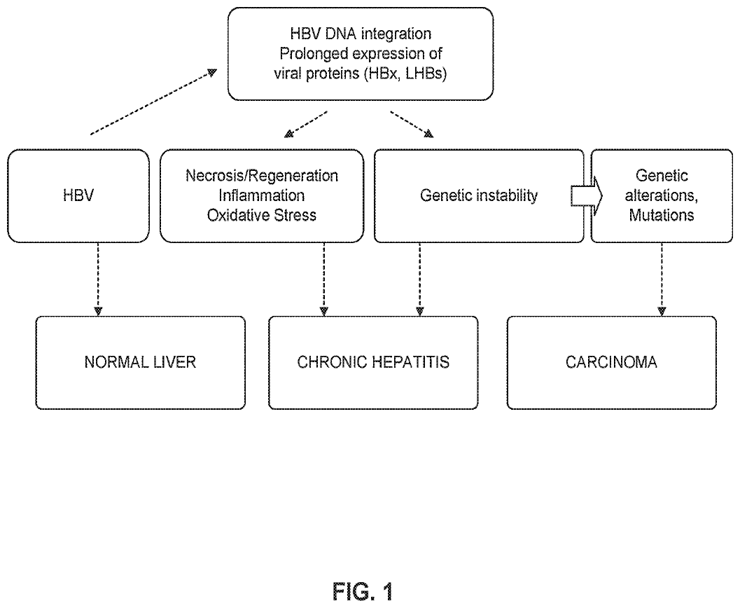

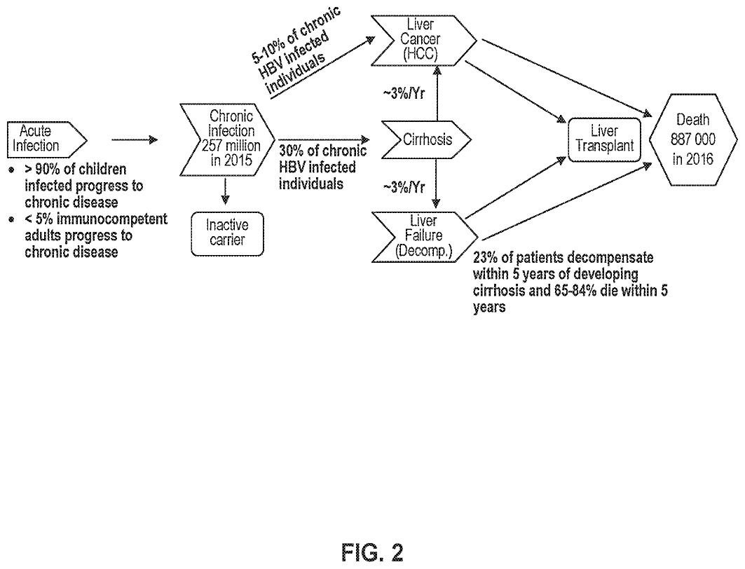

Most people do not experience any symptoms during the acute infection phase. However, some people have acute illness with symptoms that last several weeks, including yellowing of the skin and eyes (jaundice), dark urine, extreme fatigue, nausea, vomiting and abdominal pain. A small subset of persons with acute hepatitis can develop acute liver failure, which can lead to death. In some people, the hepatitis B virus can also cause a chronic liver infection that can later develop into cirrhosis (a scarring of the liver) or liver cancer. The likelihood that infection becomes chronic depends upon the age at which a person becomes infected. Children less than 6 years of age who become infected with the hepatitis B virus are the most likely to develop chronic infections. 80-90% of infants infected during the first year of life develop chronic infections; and 30-50% of children infected before the age of 6 years develop chronic infections. In adults, less than 5% of otherwise healthy persons who are infected as adults will develop chronic infection; and 20-30% of adults who are chronically infected will develop cirrhosis and/or liver cancer (FIG. 1 and FIG. 2).

There is no specific treatment for acute hepatitis B. Therefore, care is aimed at maintaining comfort and adequate nutritional balance, including replacement of fluids lost from vomiting and diarrhea. Chronic hepatitis B infection can be treated with medicines, including oral antiviral agents. Treatment (e.g., liver transplant or IFN-α nucleotide analogs) can slow the progression of cirrhosis, reduce incidence of liver cancer and improve long term survival. Recent more advanced therapeutic vaccines (i.e., GS-4777, Gilead; ABX203, Abivax) have failed in clinical trial phase 2/3. In most people, however, the treatment does not cure hepatitis B infection, but only suppresses the replication of the virus. Therefore, most people who start hepatitis B treatment must continue it for life. There is still limited access to diagnosis and treatment of hepatitis B in many resource-constrained settings.

An estimated 257 million people are living with chronic hepatitis B virus (HBV) infection (positive confirmation through surface antigen detection). In 2015, hepatitis B infection resulted in 887,000 deaths, with liver failure or liver cancer as the leading cause of death. Currently, HBV vaccines prevent infection in 95% of the cases, thereby preventing infection, liver cancer, and chronic diseases due to hepatitis B. However, there is still a large need for developing a HBV vaccine with broad coverage against multiple HBV subtypes and functionality against liver cancer. The present disclosure relates to novel HBV vaccine antigens based on bioinformatics methods and protein engineering approaches. These novel HBV vaccine candidates can be used as therapeutic vaccines against HBV and HBV-related diseases.

### Treatment Options

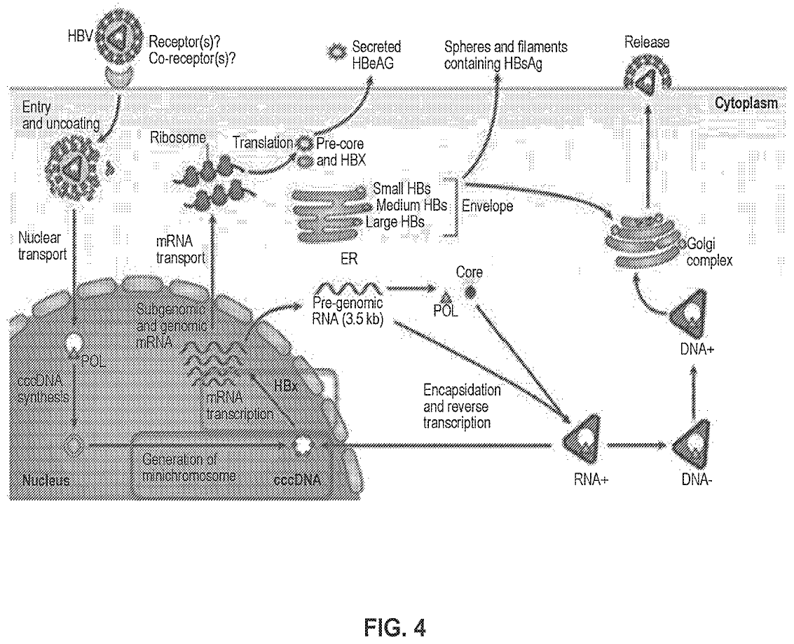

Standard of care (SOC) for chronic hepatitis B infection include PEG-IFN, and/or nucleotide analogues. However, cccDNA and immune tolerance and exhaustion can persist (FIG. 4). Infection with HBV causes hepatitis that can result in cirrhosis, liver failure and hepatocellular carcinoma (HCC). The diagnosis of HBV is based on the serological findings. In fact, viral DNA, antigens and their respective antibodies can be found in the serum. HBV is subdivided into four major serotypes (adr, adw, ayr, ayw) based on a based on a common antigenic determinant (a) and two mutually exclusive determinant pairs (d/y and w/r) found on the HBsAg. There exist ten known genotypes (A-J) and forty known subgenotypes according to overall nucleotide sequence variation of the genome. The genotypes have a distinct geographical distribution and different genotypes are associated with different disease severity, course and likelihood of complications, and response to treatment and possibly vaccination. The serotypes and genotypes do not necessarily correspond (e.g., genotype D has 10 subgenotypes).

Currently, there is no cure for chronic HBV infection. Treatment options are aimed at slowing the progression of cirrhosis and viral replication, reducing the incidence of HCC and liver failures. Current treatments are divided into two major categories: (1) immune modulator drugs, i.e., mainly type I interferon (interferon alpha and pegylated interferon alpha) designed to boost the immune system to fight viral infected cells; and (2) antiviral drugs, which include nucleoside analogues (lamivudine, entecavir and telbivudine) and nucleotide analogues (adefovir, dipivoxil and tenofovir), and aimed at interfering with viral replication. The death toll to HBV infection currently is almost 0.7 million/year on par with HIV and tuberculosis. Although the rate of new HBV infection is decreasing, the increase in overall death from hepatitis requires urgent need of the development of new treatment options.

### Therapeutic Approaches

HBV epitopes from surface (S), core (C), and polymerase (Pol) proteins are targeted by T cells during infection which mediate cellular immune responses to HBV. The HBV X protein (HBx), which includes MHC-I and MHC-II epitopes, is a multifunctional regulatory protein involved in viral pathogenesis and carcinogenesis. The HBV vaccine designs described herein include all major components that have potential T cell epitopes. Specifically, provided herein are the HBV vaccines comprising two different unique designs with genetic modifications (in the form of point mutations) and truncations in Pol, Core, Env and HBx. Also provided herein are uniquely designed multi-epitope constructs (i.e., cytotoxic T lymphocytes) with specific peptides grafted onto a human protein scaffold and linked by charged dipeptide that can stimulate the cellular immune responses required for controlling and clearing HBV infection.

Provided herein are various gene therapeutic approaches to treat HBV infection. For example, described in Table 1 are some of the strategies used to improve the immune system-mediated control of HBV. Also provided herein is an HBV therapeutic vaccine, intelligent design of a prime/boost DNA-based vaccine encompassing the combination of immunogenic regions of the main component of the HBV (HBsAg, HBcAg, HBxAg, HBeAg and HB PolAg) for priming and a combination of agonist CD8 T cell epitope for boosting.

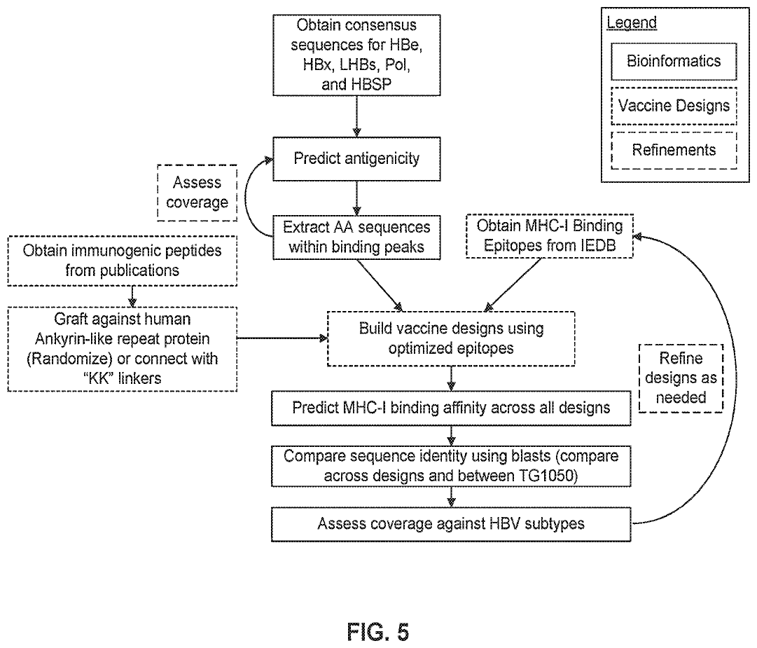

Provided herein are compositions, kits, and systems comprising and methods of making HBV recombinant vaccines. The HBV recombinant vaccines (e.g., HBV designs 1-5) in the present disclosure are engineered through protein engineering of HBe, HBx, LHBs, Pol, and HBSP. Each HBV vaccine antigen design was inspired via inventor-selected combinatorial guidance using bioinformatics analyses and in silico protein engineering methods (e.g., selection of antigenic sequences that match closer to consensus sequences, antigenicity predictions, and T cell epitope mapping, which could lead to MHC-I binding and cytokine production following T cell activation). The overall workflow of the presently disclosed HBV vaccine designs is shown in FIG. 5, and further detailed in Example 1. The present disclosure provides five HBV antigen designs (HBV designs 1-5) constructed in a multi-deleted gorilla adenovector (GC46).

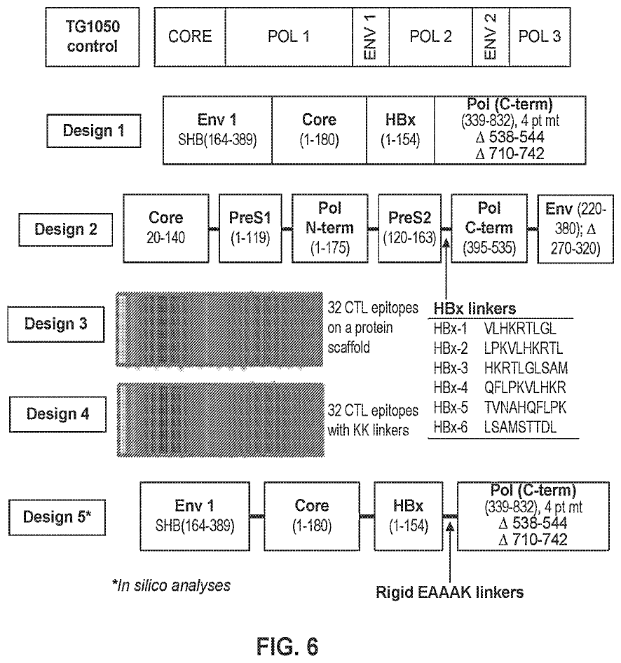

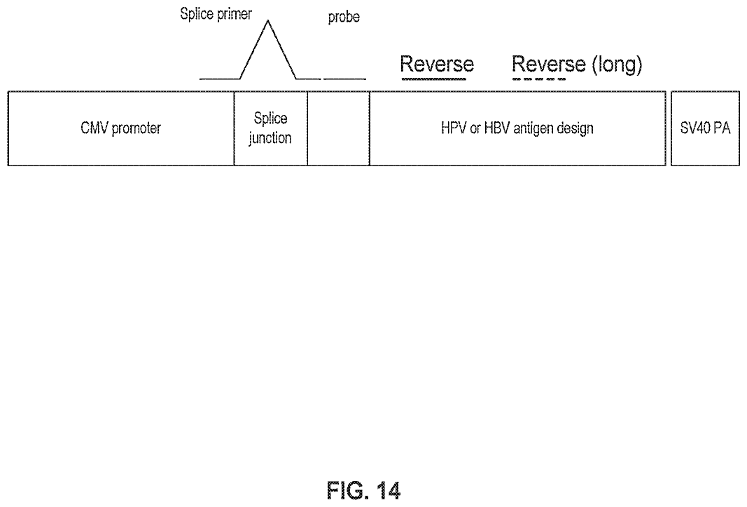

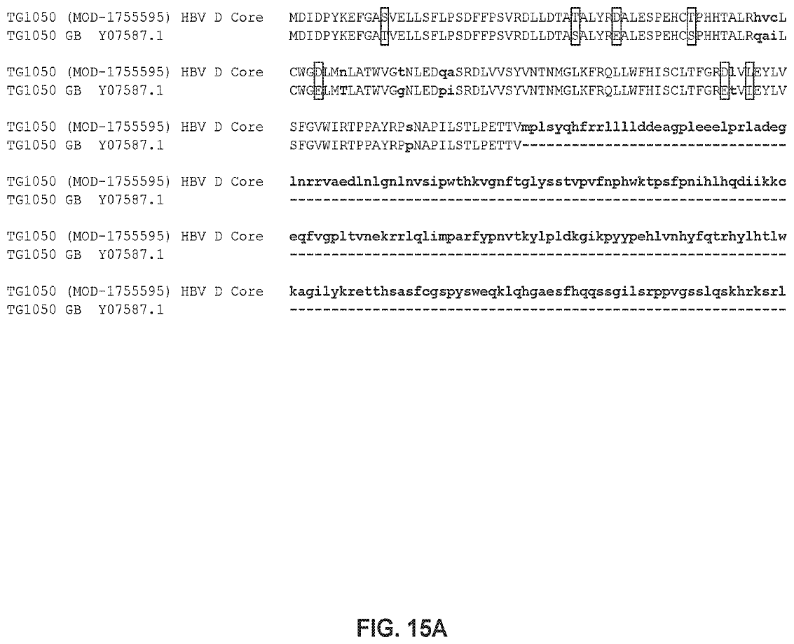

Four antigen designs (HBV designs 1-4) and a control antigen were synthesized and cloned into expression plasmids—pAdShuttles for adenovector construction (FIG. 6). HBV design was analyzed to assess whether different linkers cause prediction of different epitopes. Initial antigen screening evaluated in vitro antigen expression in transient transfection, in vitro antigen processing and presentation in transient transfection studies of monocyte-derived dendritic cells. As shown in FIG. 6, HBV designs 1 and 2 were designed based on clade D consensus. 32 HBV peptides from Core (8), Surface (8), Polymerase (8), HBx (6) and HBPS (2) were curated from literature that have experimental and functional data, such as immunogenicity data, Mass-spec. etc. (Table 4). For HBV design 3, human ankyrin-like repeat (ALR) protein scaffold (PDB code 1QYM) peptides were grafted at the helical and loop regions in a tandem manner. Two ALR scaffolds were used, connected by a cleavable linker VSQTSKLTR (SEQ ID NO: 111). ALR proteins have generally high expression and high stability. Thus, ALR proteins were used as a scaffold for the HBV peptides to create novel CTLs. HBV design 4 epitopes were separatedby KK linkers (FIG. 6). For RNA qPCR relative expression assay, 5′-TGCCAAGAGTGACGTGTCCA-3′ (SEQ ID NO: 110) was used as a splice primer, and 5′-CCCAGGTCCAACTGCAGCCGG-3′ (SEQ TD NO: 128) was used as a splice probe. Specific primers designed for each antigen were used as reverse primers (FIG. 14).

TABLE 1
Strategies to Improve the Immune Mediated Control of HBV

| Characteristics of | | | |
|---|---|---|---|
| chronic HBV | | | |
| infection | Therapeutic goals | Tools | Expected effects |
| High viral load | To decrease viral | Nucleoside analogs | Transient restoration |
| | and antigen loads | | of T-cell functions |
| High levels of PD-1 | To decrease the | Anti-PD-L1 | Restoration of |
| expression on | inhibitory signal | antibody | T-cell functions |
| HBV-specific T cells | | | |
| HBV-specific T- | To reactivate/activate | Vaccines | Reactivation of Th1 responses |
| cell exhaustion | functional HBV- | Novel adjuvants | De novo activation of CD4 |
| | specific T cells in vivo | DNA vaccines | and CD8 T cells |
| | To activate T cells ex vivo | Viral vaccines | Improvement of antigen |
| | | Ag/Ab IC | presentation |
| | | Cytokines | T-cell expansion |
| | | Modified DCs | Improvement of antigen |
| | | Stimulation of PBMCs | presentation |
| | | | Activation of killer cells |
| HBV-specific T- | To engineer new | TCR-redirected T | Non exhausted, functional |
| cell deletion | HBV-specific T cells | cells | HBV-specific T cells |
| | | | Recognition of HBV-infected cells |
| Suppressive | To target | Liver gene therapy | Elimination of HBV-infected |
| environment | functional non | | hepatocytes |
| | HBV-specific T | | |
| | cells to the liver | | |

### Delivery System

### Gorilla Adenovirus Shuttle Vector

Certain aspects of the present disclosure are directed to a vector comprising a polynucleotide encoding a polypeptide comprising one or more immune response-inducing HBV polypeptides. In certain embodiments, the vector is a viral vector. In particular embodiments, the vector is an adenoviral vector. Adenoviruses are generally associated with benign pathologies in humans, and the genomes of adenoviruses isolated from a variety of species, including humans, have been extensively studied. Adenovirus is a medium-sized (90-100 nm), non-enveloped icosahedral virus containing approximately 36 kb of double-stranded DNA. The adenovirus capsid mediates the key interactions of the early stages of the infection of a cell by the virus, and is required for packaging adenovirus genomes at the end of the adenovirus life cycle. The capsid comprises 252 capsomeres, which includes 240 hexons, 12 penton base proteins, and 12 fibers (Ginsberg et al., Virology, 28: 782-83 (1966)). The hexon comprises three identical proteins, namely polypeptide II (Roberts et al., Science, 232: 1148-51 (1986)). The penton base comprises five identical proteins and the fiber comprises three identical proteins. Proteins IIIa, VI, and IX are present in the adenoviral coat and are believed to stabilize the viral capsid (Stewart et al., Cell, 67: 145-54 (1991), and Stewart et al., EMBO J., 12(7): 2589-99 (1993)). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications (i.e., “gene therapy”), or for use as antigen delivery systems for vaccine applications. For example, adenoviruses can be produced in high titers (e.g., about 10¹³ particle units (pu)), and can transfer genetic material to non-replicating and replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3: 147-154 (1992)). Additionally, adenoviruses generally do not integrate into the host cell chromosome, but rather are maintained as a linear episome, thereby minimizing the likelihood that a recombinant adenovirus will interfere with normal cell function.

In some embodiments, the adenovirus described herein is isolated from a gorilla. There are four widely recognized gorilla subspecies within the two species of Eastern Gorilla (Gorilla beringei) and Western Gorilla (Gorilla gorilla). The Western Gorilla species includes the subspecies Western Lowland Gorilla (Gorilla gorilla gorilla) and Cross River Gorilla (Gorilla gorilla diehli). The Eastern Gorilla species includes the subspecies Mountain Gorilla (Gorilla beringei beringei) and Eastern Lowland Gorilla (Gorilla beringei graueri) (see, e.g., Wilson and Reeder, eds., Mammalian Species of the World, 3^{rd} ed., Johns Hopkins University Press, Baltimore, Maryland (2005)). In some embodiments, the adenovirus of the present disclosure is isolated from Mountain Gorilla (Gorilla beringei beringei).

Various Gorilla adenoviruses or adenoviral vectors are described in International Patent Application Publications WO 2013/052832; WO 2013/052811; and WO 2013 052799, each of which is herein incorporated by reference in its entirety.

The genomes of several such adenoviruses have been analyzed, and it has been determined that the adenovirus can have the nucleic acid sequence of, for example, SEQ ID NO: 21, SEQ ID NO: 22, SEQ ID NO: 23, SEQ ID NO: 24, or SEQ ID NO: 25, each of which includes a number of sub-sequences that serve to uniquely define the adenovirus, namely the nucleic acid sequences SEQ ID NOs: 1-10, and amino acid sequences SEQ ID NOs: 11-20. SEQ ID NOs: 6-10 encode the amino acid sequences of SEQ ID NOs: 16-20, respectively. SEQ ID NOs: 1-5 are a subset of the nucleic acid sequences of SEQ ID NOs: 6-10, respectively. SEQ ID NOs: 11-15 are a subset of the amino acid sequences of SEQ ID NOs: 16-20, respectively.

The adenovirus can be modified in the same manner as previously known adenoviruses to be used as an adenoviral vector, e.g., a gene delivery vehicle. The adenovirus and adenoviral vector can be replication-competent, conditionally replication-competent, or replication-deficient.

A replication-competent adenovirus or adenoviral vector can replicate in typical host cells, i.e., cells typically capable of being infected by an adenovirus. A replication-competent adenovirus or adenoviral vector can have one or more mutations as compared to the wild-type adenovirus (e.g., one or more deletions, insertions, and/or substitutions) in the adenoviral genome that do not inhibit viral replication in host cells. For example, the adenovirus or adenoviral vector can have a partial or entire deletion of the adenoviral early region known as the E3 region, which is not essential for propagation of the adenovirus or adenoviral genome.

A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., promoter. In such an embodiment, replication requires the presence or absence of specific factors that interact with the transcription control sequence. Conditionally-replicating adenoviral vectors are further described in U.S. Pat. No. 5,998,205.

A replication-deficient adenovirus or adenoviral vector is an adenovirus or adenoviral vector that requires complementation of one or more gene functions or regions of the adenoviral genome that are required for replication, as a result of, for example, a deficiency in one or more replication-essential gene function or regions, such that the adenovirus or adenoviral vector does not replicate in typical host cells, especially those in a human to be infected by the adenovirus or adenoviral vector.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions can be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

Whether the adenovirus or adenoviral vector is replication-competent or replication-deficient, the adenovirus or adenoviral vector retains at least a portion of the adenoviral genome. The adenovirus or adenoviral vector can comprise any portion of the adenoviral genome, including protein coding and non-protein coding regions. Desirably, the adenovirus or adenoviral vector comprises at least one nucleic acid sequence that encodes an adenovirus protein. The adenovirus or adenoviral vector can comprise a nucleic acid sequence that encodes any suitable adenovirus protein, such as, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions).

The adenovirus or adenoviral vector desirably comprises one or more nucleic acid sequences that encode the pIX protein, the DNA polymerase protein, the penton protein, the hexon protein, and/or the fiber protein. The adenovirus or adenoviral vector can comprise a full-length nucleic acid sequence that encodes a full-length amino acid sequence of an adenovirus protein. Alternatively, the adenovirus or adenoviral vector can comprise a portion of a full-length nucleic acid sequence that encodes a portion of a full-length amino acid sequence of an adenovirus protein.

A “portion” of a nucleic acid sequence comprises at least ten nucleotides (e.g., about 10 to about 5000 nucleotides). Preferably, a “portion” of a nucleic acid sequence comprises 10 or more (e.g., 15 or more, 20 or more, 25 or more, 30 or more, 35 or more, 40 or more, 45 or more, 50 or more, or 100 or more) nucleotides, but less than 5,000 (e.g., 4900 or less, 4000 or less, 3000 or less, 2000 or less, 1000 or less, 800 or less, 500 or less, 300 or less, or 100 or less) nucleotides. Preferably, a portion of a nucleic acid sequence is about 10 to about 3500 nucleotides (e.g., about 10, 20, 30, 50, 100, 300, 500, 700, 1000, 1500, 2000, 2500, or 3000 nucleotides), about 10 to about 1000 nucleotides (e.g., about 25, 55, 125, 325, 525, 725, or 925 nucleotides), or about 10 to about 500 nucleotides (e.g., about 15, 30, 40, 50, 60, 70, 80, 90, 150, 175, 250, 275, 350, 375, 450, 475, 480, 490, 495, or 499 nucleotides), or a range defined by any two of the foregoing values. More preferably, a “portion” of a nucleic acid sequence comprises no more than about 3200 nucleotides (e.g., about 10 to about 3200 nucleotides, about 10 to about 3000 nucleotides, or about 30 to about 500 nucleotides, or a range defined by any two of the foregoing values).

A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The adenovirus pIX protein is present in the adenovirus capsid, has been shown to strengthen hexon nonamer interactions, and is essential for the packaging of full-length genomes (see, e.g., Boulanger et al., J. Gen. Virol., 44: 783-800 (1979); Horwitz M. S., “Adenoviridae and their replication” in Virology, 2^{nd} ed., B. N. Fields et al. (eds.), Raven Press, Ltd., New York, pp. 1679-1721 (1990), Ghosh-Choudhury et al., EMBO J., 6: 1733-1739 (1987), and van Oostrum et al, J. Virol., 56: 439-448 (1985)). In addition to its contribution to adenovirus structure, pIX also has been shown to exhibit transcriptional properties, such as stimulation of adenovirus major late promoter (MLP) activity (see, e.g., Lutz et al., J. Virol., 71(7): 5102-5109 (1997)). Nucleic acid sequences that encode all or a portion of an adenovirus pIX protein include, for example, SEQ ID NO: 6 and SEQ ID NO: 1. Amino acid sequences that comprise a full-length pIX protein, or a portion thereof, include, for example, SEQ ID NO: 16 and SEQ ID NO: 11.

The adenovirus DNA polymerase protein is essential for viral DNA replication both in vitro and in vivo. The polymerase co-purifies in a complex with the precursor (pTP) of the terminal protein (TP), which is covalently attached to the 5′ ends of adenovirus DNA (Field et al., J. Biol. Chem., 259: 9487-9495 (1984)). Both the adenovirus DNA polymerase and pTP are encoded by the E2 region. The polymerase protein is required for the expression of all the structural proteins except for pIX. Without the gene sequence for polymerase protein, polymerase protein is not produced. As a result, the viral genome is not replicated, the Major Late Promoter is not activated, and the capsid proteins are not expressed. Nucleic acid sequences that encode all or a portion of an adenovirus DNA polymerase protein include, for example, SEQ ID NO: 7 and SEQ ID NO: 2. Amino acid sequences that comprise a full-length adenovirus DNA polymerase, or a portion thereof, include, for example, SEQ ID NO: 17 and SEQ ID NO: 12.

The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, hexon is a primary target for modification in order to reduce neutralization of adenoviral vectors (see, e.g., Gall et al., J. Virol., 72: 10260-264 (1998), and Rux et al., J. Virol., 77(17): 9553-9566 (2003)). The major structural features of the hexon protein are shared by adenoviruses across serotypes, but the hexon protein differs in size and immunological properties between serotypes (Jornvall et al., J. Biol. Chem., 256(12): 6181-6186 (1981)). A comparison of 15 adenovirus hexon proteins revealed that the predominant antigenic and serotype-specific regions of the hexon appear to be in loops 1 and 2 (i.e., L1 or 11, and L11 or 12, respectively), within which are seven discrete hypervariable regions (HVR1 to HVR7) varying in length and sequence between adenoviral serotypes (Crawford-Miksza et al., J. Virol., 70(3): 1836-1844 (1996)). Nucleic acid sequences that encode all or a portion of an adenovirus hexon protein include, for example, SEQ ID NO: 9 and SEQ ID NO: 4. Amino acid sequences that comprise a full-length adenovirus hexon protein, or a portion thereof, include, for example, SEQ ID NO: 19 and SEQ ID NO: 14.

The adenovirus fiber protein is a homotrimer of the adenoviral polypeptide IV that has three domains: the tail, shaft, and knob. (Devaux et al., J. Molec. Biol., 215: 567-88 (1990), Yeh et al., Virus Res., 33: 179-98 (1991)). The fiber protein mediates primary viral binding to receptors on the cell surface via the knob and the shaft domains (Henry et al., J. Virol., 68(8): 5239-46 (1994)). The amino acid sequences for trimerization are located in the knob, which appears necessary for the amino terminus of the fiber (the tail) to properly associate with the penton base (Novelli et al., Virology, 185: 365-76 (1991)). In addition to recognizing cell receptors and binding the penton base, the fiber contributes to serotype identity. Fiber proteins from different adenoviral serotypes differ considerably (see, e.g., Green et al., EMBO J., 2: 1357-65 (1983), Chroboczek et al., Virology, 186: 280-85 (1992), and Signas et al., J. Virol., 53: 672-78 (1985)). Thus, the fiber protein has multiple functions key to the life cycle of adenovirus. Nucleic acid sequences that encode all or a portion of an adenovirus fiber protein include, for example, SEQ ID NO: 10 and SEQ ID NO: 5. Amino acid sequences that comprise a full-length adenovirus fiber protein, or a portion thereof, include, for example, SEQ ID NO: 20 and SEQ ID NO: 15.

The adenovirus penton base protein is located at the vertices of the icosahedral capsid and comprises five identical monomers. The penton base protein provides a structure for bridging the hexon proteins on multiple facets of the icosahedral capsid, and provides the essential interface for the fiber protein to be incorporated in the capsid. Each monomer of the penton base contains an RGD tripeptide motif (Neumann et al., Gene, 69: 153-157 (1988)). The RGD tripeptide mediates binding to αv integrins and adenoviruses that have point mutations in the RGD sequence of the penton base are restricted in their ability to infect cells (Bai et al., J. Virol., 67: 5198-5205 (1993)). Thus, the penton base protein is essential for the architecture of the capsid and for maximum efficiency of virus-cell interaction. Nucleic acid sequences that encode all or a portion of an adenovirus penton base protein include, for example, SEQ ID NO: 8 and SEQ ID NO: 3. Amino acid sequences that comprise a full-length adenovirus penton base protein, or a portion thereof, include, for example, SEQ ID NO: 18 and SEQ ID NO: 13.

Nucleic acid or amino acid sequence “identity,” as described herein, can be determined by comparing a nucleic acid or amino acid sequence of interest to a reference nucleic acid or amino acid sequence. The numbers of nucleotides or amino acid residues that have been changed and/or modified (such as, e.g., by point mutations, insertions, or deletions) in the reference sequence so as to result in the sequence of interest are counted. The total number of such changes is subtracted from the total length of the sequence of interest, and the difference is divided by the length of the sequence of interest and expressed as a percentage. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The adenovirus or adenoviral vector can comprise one, two, three, four, or all five of the aforementioned sequences alone or in any combination. In this respect, the adenovirus or adenoviral vector can comprise any combination of any two of the aforementioned sequences, any combination of any three of the aforementioned sequences, any combination of any four of the aforementioned sequences, or all five of the aforementioned sequences.

As discussed herein, the adenovirus or adenoviral vector can be replication-competent, conditionally-replicating, or replication-deficient. Preferably, the adenovirus or adenoviral vector is replication-deficient, such that the replication-deficient adenovirus or adenoviral vector requires complementation of at least one replication-essential gene function of one or more regions of the adenoviral genome for propagation (e.g., to form adenoviral vector particles).

The replication-deficient adenovirus or adenoviral vector can be modified in any suitable manner to cause the deficiencies in the one or more replication-essential gene functions in one or more regions of the adenoviral genome for propagation. The complementation of the deficiencies in the one or more replication-essential gene functions of one or more regions of the adenoviral genome refers to the use of exogenous means to provide the deficient replication-essential gene functions. Such complementation can be effected in any suitable manner, for example, by using complementing cells and/or exogenous DNA (e.g., helper adenovirus) encoding the disrupted replication-essential gene functions.

The adenovirus or adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad). See Morsy et al., Proc. Natl. Acad. Sci. USA, 95: 965-976 (1998); Chen et al., Proc. Natl. Acad. Sci. USA, 94: 1645-1650 (1997); and Kochanek et al., Hum. Gene Ther., 10: 2451-2459 (1999). Examples of replication-deficient adenoviral vectors are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the E1B subregion of the adenoviral genome for the adenovirus or adenoviral vector to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus or adenoviral vector to propagate (e.g., to form adenoviral vector particles).

The E3 region does not include any replication-essential gene functions, such that a deletion of the E3 region in part or in whole does not require complementation of any gene functions in the E3 region for the adenovirus or adenoviral vector to propagate (e.g., to form adenoviral vector particles). In the context of the present disclosure, the E3 region is defined as the region that initiates with the open reading frame that encodes a protein with high homology to the 12.5K protein from the E3 region of human adenovirus 5 (NCBI reference sequence AP_000218) and ends with the open reading frame that encodes a protein with high homology to the 14.7K protein from the E3 region of human adenovirus 5 (NCBI reference sequence AP_000224.1). The E3 region can be deleted in whole or in part, or retained in whole or in part. The size of the deletion can be tailored so as to retain an adenovirus or adenoviral vector whose genome closely matches the optimum genome packaging size. A larger deletion will accommodate the insertion of larger heterologous nucleic acid sequences in the adenovirus or adenoviral genome. In one embodiment of the present disclosure, the L4 polyadenylation signal sequences, which reside in the E3 region, are retained.

The E4 region comprises multiple open reading frames (ORFs). An adenovirus or adenoviral vector with a deletion of all of the open reading frames of the E4 region except ORF6, and in some cases ORF3, does not require complementation of any gene functions in the E4 region for the adenovirus or adenoviral vector to propagate (e.g., to form adenoviral vector particles). Conversely, an adenovirus or adenoviral vector with a disruption or deletion of ORF6, and in some cases ORF3, of the E4 region (e.g., with a deficiency in a replication-essential gene function based in ORF6 and/or ORF3 of the E4 region), with or without a disruption or deletion of any of the other open reading frames of the E4 region or the native E4 promoter, polyadenylation sequence, and/or the right-side inverted terminal repeat (ITR), requires complementation of the E4 region (specifically, of ORF6 and/or ORF3 of the E4 region) for the adenovirus or adenoviral vector to propagate (e.g., to form adenoviral vector particles). The late regions of the adenoviral genome include the L1, L2, L3, L4, and L5 regions. The adenovirus or adenoviral vector also can have a mutation in the major late promoter (MLP), as discussed in International Patent Application Publication WO 2000/000628, which can render the adenovirus or adenoviral vector replication-deficient if desired.

The one or more regions of the adenoviral genome that contain one or more deficiencies in replication-essential gene functions desirably are one or more early regions of the adenoviral genome, i.e., the E1, E2, and/or E4 regions, optionally with the deletion in part or in whole of the E3 region.

The replication-deficient adenovirus or adenoviral vector also can have one or more mutations as compared to the wild-type adenovirus (e.g., one or more deletions, insertions, and/or substitutions) in the adenoviral genome that do not inhibit viral replication in host cells. Thus, in addition to one or more deficiencies in replication-essential gene functions, the adenovirus or adenoviral vector can be deficient in other respects that are not replication-essential. For example, the adenovirus or adenoviral vector can have a partial or entire deletion of the adenoviral early region known as the E3 region, which is not essential for propagation of the adenovirus or adenoviral genome.

In one embodiment, the adenovirus or adenoviral vector is replication-deficient and requires, at most, complementation of the E1 region or the E4 region of the adenoviral genome, for propagation (e.g., to form adenoviral vector particles). Thus, the replication-deficient adenovirus or adenoviral vector requires complementation of at least one replication-essential gene function of the E1A subregion and/or the E1B region of the adenoviral genome (denoted an E1-deficient adenoviral vector) or the E4 region of the adenoviral genome (denoted an E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome and at least one gene function of the nonessential E3 region of the adenoviral genome (denoted an E1/E3-deficient adenoviral vector). The adenovirus or adenoviral vector can be deficient in at least one replication-essential gene function (desirably all replication-essential gene functions) of the E4 region of the adenoviral genome and at least one gene function of the nonessential E3 region of the adenoviral genome (denoted an E3/E4-deficient adenoviral vector).

In one embodiment, the adenovirus or adenoviral vector is replication-deficient and requires, at most, complementation of the E2 region, preferably the E2A subregion, of the adenoviral genome, for propagation (e.g., to form adenoviral vector particles). Thus, the replication-deficient adenovirus or adenoviral vector requires complementation of at least one replication-essential gene function of the E2A subregion of the adenoviral genome (denoted an E2A-deficient adenoviral vector) for propagation (e.g., to form adenoviral vector particles). The adenovirus or adenoviral vector can be deficient in at least one replication-essential gene function (desirably all replication-essential gene functions) of the E2A region of the adenoviral genome and at least one gene function of the nonessential E3 region of the adenoviral genome (denoted an E2A/E3-deficient adenoviral vector).

In one embodiment, the adenovirus or adenoviral vector is replication-deficient and requires, at most, complementation of the E1 and E4 regions of the adenoviral genome for propagation (e.g., to form adenoviral vector particles). Thus, the replication-deficient adenovirus or adenoviral vector requires complementation of at least one replication-essential gene function of both the E1 and E4 regions of the adenoviral genome (denoted an E1/E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, at least one replication-essential gene function of the E4 region of the adenoviral genome, and at least one gene function of the nonessential E3 region of the adenoviral genome (denoted an E1/E3/E4-deficient adenoviral vector). The adenovirus or adenoviral vector preferably requires, at most, complementation of the E1 region of the adenoviral genome for propagation, and does not require complementation of any other deficiency of the adenoviral genome for propagation. More preferably, the adenovirus or adenoviral vector requires, at most, complementation of the E1 and E4 regions of the adenoviral genome for propagation, and does not require complementation of any other deficiency of the adenoviral genome for propagation.

The adenovirus or adenoviral vector, when deficient in multiple replication-essential gene functions of the adenoviral genome (e.g., an E1/E4-deficient adenoviral vector), can include a spacer sequence to provide viral growth in a complementing cell line similar to that achieved by adenoviruses or adenoviral vectors deficient in a single replication-essential gene function (e.g., an E1-deficient adenoviral vector). The spacer sequence can contain any nucleotide sequence or sequences which are of a desired length, such as sequences at least about 15 base pairs (e.g., between about 15 nucleotides and about 12,000 nucleotides), preferably about 100 nucleotides to about 10,000 nucleotides, more preferably about 500 nucleotides to about 8,000 nucleotides, even more preferably about 1,500 nucleotides to about 6,000 nucleotides, and most preferably about 2,000 to about 3,000 nucleotides in length, or a range defined by any two of the foregoing values. The spacer sequence can be coding or non-coding and native or non-native with respect to the adenoviral genome, but does not restore the replication-essential function to the deficient region. The spacer also can contain an expression cassette. More preferably, the spacer comprises a polyadenylation sequence and/or a gene that is non-native with respect to the adenovirus or adenoviral vector. The use of a spacer in an adenoviral vector is further described in, for example, U.S. Pat. No. 5,851,806 and International Patent Application Publication WO 1997/021826.

By removing all or part of the adenoviral genome, for example, the E1, E3, and E4 regions of the adenoviral genome, the resulting adenovirus or adenoviral vector is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of adenovirus or the adenoviral vector particle. The exogenous nucleic acid sequence preferably is positioned in the E1 region, the E3 region, or the E4 region of the adenoviral genome.

The replication-deficient adenovirus or adenoviral vector of the present disclosure can be produced in complementing cell lines that provide gene functions not present in the replication-deficient adenovirus or adenoviral vector, but required for viral propagation, at appropriate levels in order to generate high titers of viral vector stock. Such complementing cell lines are known and include, but are not limited to, 293 cells (described in, e.g., Graham et al., J. Gen. Virol., 36: 59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 95/34671 and Brough et al., J. Virol., 71: 9206-9213 (1997)). Other suitable complementing cell lines to produce the replication-deficient adenovirus or adenoviral vector of the present disclosure include complementing cells that have been generated to propagate adenoviral vectors encoding transgenes whose expression inhibits viral growth in host cells (see, e.g., U.S. Patent Application Publication No. 2008/0233650). Additional suitable complementing cells are described in, for example, U.S. Pat. Nos. 6,677,156 and 6,682,929, and International Patent Application Publication WO 2003/020879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the replication-deficient adenovirus or adenoviral vector. Alternatively, the inventive adenovirus or adenoviral vector can comprise a non-native replication-essential gene that complements for the one or more replication-essential gene functions lacking in the inventive replication-deficient adenovirus or adenoviral vector. For example, an E1/E4-deficient adenoviral vector can be engineered to contain a nucleic acid sequence encoding E4 ORF 6 that is obtained or derived from a different adenovirus (e.g., an adenovirus of a different serotype than the inventive adenovirus or adenoviral vector, or an adenovirus of a different species than the inventive adenovirus or adenoviral vector).

The adenovirus or adenoviral vector can further comprise a transgene. The term “transgene” is defined herein as a non-native nucleic acid sequence that is operably linked to appropriate regulatory elements (e.g., a promoter), such that the non-native nucleic acid sequence can be expressed to produce a protein (e.g., peptide or polypeptide). The regulatory elements (e.g., promoter) can be native or non-native to the adenovirus or adenoviral vector.

A “non-native” nucleic acid sequence is any nucleic acid sequence (e.g., DNA, RNA, or cDNA sequence) that is not a naturally occurring nucleic acid sequence of an adenovirus in a naturally occurring position. Thus, the non-native nucleic acid sequence can be naturally found in an adenovirus, but located at a non-native position within the adenoviral genome and/or operably linked to a non-native promoter. The terms “non-native nucleic acid sequence,” “heterologous nucleic acid sequence,” and “exogenous nucleic acid sequence” are synonymous and can be used interchangeably in the context of the present disclosure. The non-native nucleic acid sequence preferably is DNA and preferably encodes a protein (i.e., one or more nucleic acid sequences encoding one or more proteins).

The non-native nucleic acid sequence can encode a therapeutic protein that can be used to prophylactically or therapeutically treat a mammal for a disease. Examples of suitable therapeutic proteins include cytokines, toxins, tumor suppressor proteins, growth factors, hormones, receptors, mitogens, immunoglobulins, neuropeptides, neurotransmitters, and enzymes. Alternatively, the non-native nucleic acid sequence can encode an antigen of a pathogen (e.g., a bacterium or a virus), and the adenovirus or adenoviral vector can be used as a vaccine.

### Viral Based Delivery System

The present disclosure also provides delivery systems, such as viral-based systems, in which a nucleic acid described herein is inserted. Representative viral expression vectors include, but are not limited to, adeno-associated viral vectors, adenovirus-based vectors, lentivirus-based vectors, retroviral vectors, and herpes virus-based vectors. In an embodiment, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In an additional embodiment, the viral vector is an adeno-associated viral vector. In a further embodiment, the viral vector is a retroviral vector. In general, and in embodiments, a suitable vector contains an origin of replication functional in at least one organism, a promoter sequence, convenient restriction endonuclease sites, and one or more selectable markers.

Additional suitable vectors include integrating expression vectors, which can randomly integrate into the host cell's DNA, or can include a recombination site to enable the specific recombination between the expression vector and the host cell's chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNATM5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes can include genes encoding luciferase, beta-galactosidase, chloramphenicol acetyl transferase, secreted alkaline phosphatase, or the green fluorescent protein gene (e.g., Ui-Tei et al., FEBS Letters 479: 79-82 (2000)). Suitable expression systems are well known and can be prepared using known techniques or obtained commercially. In general, the construct with the minimal 5′ flanking region showing the highest level of expression of reporter gene is identified as the promoter. Such promoter regions can be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (Molecular Cloning: A Laboratory Manual, Cold Spring Harbor Laboratory, New York (2001)). In embodiments, a method for the introduction of a polynucleotide into a host cell is calcium phosphate transfection or polyethylenimine (PEI) Transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### Non-Viral Based Delivery System

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

In some instances, polynucleotides encoding polypeptides can also be introduced into cells using non-viral based delivery systems, such as the “Sleeping Beauty (SB) Transposon System,” which refers a synthetic DNA transposon system for introducing DNA sequences into the chromosomes of vertebrates. Some exemplary embodiments of the system are described, for example, in U.S. Pat. Nos. 6,489,458 and 8,227,432. The Sleeping Beauty transposon system is composed of a Sleeping Beauty (SB) transposase and a SB transposon. In embodiments, the Sleeping Beauty transposon system can include the SB11 transposon system, the SB100X transposon system, or the SB110 transposon system.

DNA transposons translocate from one DNA site to another in a simple, cut-and-paste manner. Transposition is a precise process in which a defined DNA segment is excised from one DNA molecule and moved to another site in the same or different DNA molecule or genome. As do other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, for instance a DNA or mRNA source, in which case the transposon becomes a non-autonomous element. Non-autonomous transposons are most useful as genetic tools because after insertion they cannot independently continue to excise and re-insert. SB transposons envisaged to be used as non-viral vectors for introduction of genes into genomes of vertebrate animals and for gene therapy.

Regardless of the method used to introduce exogenous nucleic acids into a host cell or otherwise expose a cell to the inhibitor of the present disclosure, in order to confirm the presence of the recombinant DNA sequence in the host cell, a variety of assays can be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

In embodiments, a modified effector cell described herein and other genetic elements are delivered to a cell using the SB11 transposon system, the SB100X transposon system, the SB110 transposon system, the piggyBac transposon system (see, e.g., Wilson et al, “PiggyBac Transposon-mediated Gene Transfer in Human Cells,” Molecular Therapy 15:139-145 (2007), incorporated herein by reference in its entirety) and/or the piggyBac transposon system (see, e.g., Mitra et al., “Functional characterization of piggyBac from the bat Myotis lucifugus unveils an active mammalian DNA transposon,” Proc. Natl. Acad. Sci USA 110:234-239 (2013). Additional transposases and transposon systems are provided in U.S. Pat. Nos. 6,489,458; 6,613,752, 7,148,203; 7,985,739; 8,227,432; 9,228,180; U.S. Patent Publn. No. 2011/0117072; Mates et al., Nat Genet., 41(6):753-61 (2009). doi: 10.1038/ng.343. Epub 2009 May 3, Gene Ther., 18(9):849-56 (2011). doi: 10.1038/gt.2011.40. Epub 2011 Mar. 31 and in Ivics et al., Cell. 91(4):501-10, (1997), each of which is incorporated herein by reference in their entirety.

Additional suitable non-viral systems can include integrating expression vectors, which can randomly integrate into the host cell's DNA, or can include a recombination site to enable the specific recombination between the expression vector and the host cell's chromosome. Targeted integration of transgenes into predefined genetic loci is a desirable goal for many applications. First, a first recombination site for a site-specific recombinase is inserted at a genomic site, either at a random or at a predetermined location. Subsequently, the cells are transfected with a plasmid carrying the gene or DNA of interest and the second recombination site and a source for recombinase (expression plasmid, RNA, protein, or virus-expressing recombinase). Recombination between the first and second recombination sites leads to integration of plasmid DNA.

Such integrating expression vectors can utilize the endogenous expression control sequences of the host cell's chromosomes to effect expression of the desired protein. In some embodiments, targeted integration is promoted by the presence of sequences on the donor polynucleotide that are homologous to sequences flanking the integration site. For example, targeted integration using the donor polynucleotides described herein can be achieved following conventional transfection techniques, e.g. techniques used to create gene knockouts or knockins by homologous recombination. In other embodiments, targeted integration is promoted both by the presence of sequences on the donor polynucleotide that are homologous to sequences flanking the integration site, and by contacting the cells with donor polynucleotide in the presence of a site-specific recombinase. By a site-specific recombinase, or simply a recombinase, it is meant is a polypeptide that catalyzes conservative site-specific recombination between its compatible recombination sites. As used herein, a site-specific recombinase includes native polypeptides as well as derivatives, variants and/or fragments that retain activity, and native polynucleotides, derivatives, variants, and/or fragments that encode a recombinase that retains activity.

Also provided herein is a system for integrating heterologous genes in a host cell, said system comprising one or more gene expression cassettes. In some instances, the system includes a first gene expression cassette comprising a first polynucleotide encoding a first polypeptide construct. In other instances, the system can include a second gene expression cassette comprising a second polynucleotide encoding a second polypeptide construct. In yet other instances, the system can include a third gene expression cassette. In one embodiment, one of the gene expression cassettes can comprise a gene switch polynucleotide encoding one or more of: (i) a transactivation domain; (ii) nuclear receptor ligand binding domain; (iii) a DNA-binding domain; and (iv) ecdysone receptor binding domain. In another embodiment, the system further includes recombinant attachment sites; and a serine recombinase; such that upon contacting said host cell with at least said first gene expression cassette, in the presence of said serine recombinase, said heterologous genes are integrated in said host cell.

In some instances, the system further comprises a ligand; such that upon contacting said host cell, in the presence of said ligand, said heterologous gene are expressed in said host cell. In one instance, the system also includes recombinant attachment sites. In some instances, one recombination attachment site is a phage genomic recombination attachment site (attP) or a bacterial genomic recombination attachment site (attB). In one instance, the host cell is an eukaryotic cell. In another instance, the host cell is a human cell. In further instances, the host cell is a T cell or NK cell.

### Promoters

“Promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters are alcohol-regulated promoters, tetracycline-regulated promoters, steroid-regulated promoters, metal-regulated promoters, pathogenesis-regulated promoters, temperature-regulated promoters and light-regulated promoters. In one embodiment, the inducible promoter is part of a genetic switch. The inducible promoter can be a gene switch ligand inducible promoter. In some cases, an inducible promoter can be a small molecule ligand-inducible two polypeptide ecdysone receptor-based gene switch, such as RHEOSWITCH® gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919 each of which is incorporated by reference in its entirety).

Provided herein are methods comprising administering to a subject at least one non-viral vector comprising a polynucleotide encoding a polypeptide sequence described herein comprising at least two functional proteins or portions thereof; at least one promoter; and at least one engineered recombination site; wherein said at least one promoter drives expression of said at least two functional proteins. In some cases, at least one promoter can be constitutive. In some cases, at least one promoter can be tissue-specific. In some cases, at least one promoter can be inducible. In some cases, an inducible promoter is a small molecule ligand-inducible two polypeptide ecdysone receptor-based gene switch. In other cases, a combination of promoters wherein at least one promoter can be inducible and at least one promoter can be activation specific can be utilized.

An inducible promoter utilizes a ligand for dose-regulated control of expression of said at least two genes. In some cases, a ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-buty1)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof.

In some embodiments, a promoter is an inducible promoter. In some embodiments, a promoter is a non-inducible promoter. In some cases, a promoter can be a tissue-specific promoter. Herein “tissue-specific” refers to regulated expression of a gene in a subset of tissues or cell types. In some cases, a tissue-specific promoter can be regulated spatially such that the promoter drives expression only in certain tissues or cell types of an organism. In other cases, a tissue-specific promoter can be regulated temporally such that the promoter drives expression in a cell type or tissue differently across time, including during development of an organism. In some cases, a tissue-specific promoter is regulated both spatially and temporally. In certain embodiments, a tissue-specific promoter is activated in certain cell types either constitutively or intermittently at particular times or stages of the cell type. For example, a tissue-specific promoter can be a promoter that is activated when a specific cell such as a T cell or a NK cell is activated. T cells can be activated in a variety of ways, for example, when presented with peptide antigens by MHC class II molecules.

In one case, at least one promoter is an engineered promoter or variants thereof. As described herein, the promoter can incorporate minimal promoter sequences from IL-2 and one or more of the following: nuclear factor of activated T-cells (NFAT) response element(s); NFIL2D response element, NFkB/TCF response element, NF_AT/NFIL2B response element or NFIL2A/OCT response element. Examples of response elements are described in Mattila et al., EMBO J. 9(13):4425-33 (1990); incorporated herein in its entirety.

In some embodiments, at least one promoter comprises IL-2 core promoter (SEQ ID NO: 26). In one embodiment, at least one promoter comprises IL-2 minimal promoter (SEQ ID NO: 27). In another embodiment, at least one promoter comprises IL-2 enhancer and promoter variant (SEQ ID NOS: 26-28). In yet another embodiment, at least one promoter comprises NF-κB binding site (SEQ ID NOS: 30-32). In some embodiments, at least one promoter comprises (NF-κB)₁-IL2 promoter variant (SEQ ID NO: 30). In some embodiments, at least one promoter comprises (NF-κB)₃—IL2 promoter variant (SEQ ID NO: 31). In some embodiments, at least one promoter comprises (NF-κB)₆-IL2 promoter variant (SEQ ID NO: 32). In one embodiment, at least one promoter comprises 1X nuclear factor of activated T-cells (NFAT) response elements-IL2 promoter variant (SEQ ID NO: 33). In another embodiment, at least one promoter comprises 3X NFAT response element (SEQ ID NOS: 34-35). In yet another embodiment, at least one promoter comprises 6X NFAT response elements-IL2 promoter variant (SEQ ID NOS: 36-39). In some embodiments, at least one promoter comprises human EF1A1 promoter variant (SEQ ID NOS: 40-41). In some embodiment, at least one promoter comprises human EF1A1 promoter and enhancer (SEQ ID NO: 42). In some embodiments, at least one promoter comprises human UBC promoter (SEQ ID NO: 43). In some embodiments, at least one promoter comprises 6 site GAL4-inducible proximal factor binding element (PFB). In some embodiment, at least one promoter comprises synthetic minimal promoter 1 (inducible promoter) (SEQ ID NO: 44).

Use of gene switch for ligand inducible control of IL-12 expression described herein can improve the safety profile of IL-12 by for example allowing for regulated expression and improving therapeutic index. However, a condition for ligand dose dependent expression of IL-12 using gene switch(es) is the presence or absence of activator ligand (e.g. veledimex). In certain embodiments, an additional conditional control for induction of IL-12 expression is contemplated. Gene switch components under the control of T cell activated specific promoters are provided. This results in conditional expression (e.g., T cell activation) of gene switch components necessary for veledimex controlled expression of transgene(s) under control of a gene switch. In some embodiments, this results in preferential expression of cytokines such as IL-12 or IL-15 by tumor specific T cells when veledimex is present and T cells are activated. This can lead to increased localized levels of gene switch controlled transgene expression.

For example, T cell activation specific expression of gene switch components can be controlled by promoter comprising Nuclear Factor of Activated T-cells (NFAT) response element(s). NFAT transcription factors are key modulators of effector T-cell states. NFATs are early transcriptional checkpoint progressively driving exhaustion. NFATs are quickly activated in T cells following TCR stimulation and form a protein complex with AP-1 induced by appropriate co-stimulation signaling and regulate effector genes and T-cell functions. NFAT response element(s) can be fused with other minimal promoter sequences (e.g. IL2 minimal promoter) to drive expression of transgenes in response to T cell activation.

Other examples of activation specific promoters include but are not limited to interleukin-2 (IL2) promoter and Programmed Death (PD)-1 (CD279) promoter. Gene switch components can also be conditionally expressed upon immune cell activation by fusing binding sites for other nuclear factors like NF-κB of proinflammatory signaling pathway to minimal promoter sequence (e.g. IL2).

In certain embodiments, the promoter can be any one or more of: IL-2 core promoter, IL-2 minimal promoter, IL-2 enhancer and promoter variant, (NF-κB)₁-IL2 promoter variant, (NF-κB)₃—IL2 promoter variant, (NF-κB)₆-IL2 promoter variant, 1X NFAT response elements-IL2 promoter variant, 3X NFAT response elements-IL2 promoter variant, 6X NFAT response elements-IL2 promoter variant, human EEF1A1 promoter variant, human EEF1A1 promoter and enhancer, human UBC promoter and synthetic minimal promoter 1. In certain embodiments, the promoter nucleotides can comprise SEQ ID NOs: 26-44.

### Gene Switch

Provided herein are gene switch polypeptides, polynucleotides encoding ligand-inducible gene switch polypeptides, and methods and systems incorporating these polypeptides and/or polynucleotides. In certain aspects, the present disclosure is directed to a polynucleotide comprising one or more polynucleotides encoding a gene switch system for inducible control of heterologous gene expression, wherein the heterologous gene expression is regulated by said gene switch system; and, wherein said heterologous gene comprises a polynucleotide encoding a polypeptide comprising one or more immune response-inducing hepatitis B virus (HBV) polypeptides, disclosed herein.

The term “gene switch” refers to the combination of a response element associated with a promoter, and for instance, an EcR based system which, in the presence of one or more ligands, modulates the expression of a gene into which the response element and promoter are incorporated. Tightly regulated inducible gene expression systems or gene switches are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

An early version of EcR-based gene switch used Drosophila melanogaster EcR (DmEcR) and Mus musculus RXR (MmRXR) polypeptides and showed that these receptors in the presence of steroid, ponasteroneA, transactivate reporter genes in mammalian cell lines and transgenic mice (Christopherson et al., Proc. Natl. Acad. Sci. USA 89(14):6314-18 (1992); No et al., Proc. Natl. Acad. Sci. USA 93(8):3346-51 (1996)). Later, Suhr et al. (Proc. Natl. Acad. Sci. USA 95(14):7999-8004 (1998)) showed that non-steroidal ecdysone agonist, tebufenozide, induced high level of transactivation of reporter genes in mammalian cells through Bombyx mori EcR (BmEcR) in the absence of exogenous heterodimer partner.

International Patent Applications No. PCT/US97/05330 (WO 97/38117) and PCT/US99/08381 (WO99/58155) disclose methods for modulating the expression of an exogenous gene in which a DNA construct comprising the exogenous gene and an ecdysone response element is activated by a second DNA construct comprising an ecdysone receptor that, in the presence of a ligand therefor, and optionally in the presence of a receptor capable of acting as a silent partner, binds to the ecdysone response element to induce gene expression. In this example, the ecdysone receptor was isolated from Drosophila melanogaster. Typically, such systems require the presence of the silent partner, preferably retinoid X receptor (RXR), in order to provide optimum activation. In mammalian cells, insect ecdysone receptor (EcR) is capable of heterodimerizing with mammalian retinoid X receptor (RXR) and, thereby, be used to regulate expression of target genes or heterologous genes in a ligand dependent manner. International Patent Application No. PCT/US98/14215 (WO 99/02683) discloses that the ecdysone receptor isolated from the silk moth Bombyx mori is functional in mammalian systems without the need for an exogenous dimer partner.

U.S. Pat. No. 6,265,173 discloses that various members of the steroid/thyroid superfamily of receptors can combine with Drosophila melanogaster ultraspiracle receptor (USP) or fragments thereof comprising at least the dimerization domain of USP for use in a gene expression system. U.S. Pat. No. 5,880,333 discloses a Drosophila melanogaster EcR and ultraspiracle (USP) heterodimer system used in plants in which the transactivation domain and the DNA binding domain are positioned on two different hybrid proteins. In each of these cases, the transactivation domain and the DNA binding domain (either as native EcR as in International Patent Application No. PCT/US98/14215 or as modified EcR as in International Patent Application No. PCT/US97/05330) were incorporated into a single molecule and the other heterodimeric partners, either USP or RXR, were used in their native state.

International Patent Application No. PCT/US01/0905 discloses an ecdysone receptor-based inducible gene expression system in which the transactivation and DNA binding domains are separated from each other by placing them on two different proteins results in greatly reduced background activity in the absence of a ligand and significantly increased activity over background in the presence of a ligand. This two-hybrid system is a significantly improved inducible gene expression modulation system compared to the two systems disclosed in applications PCT/US97/05330 and PCT/US98/14215. The two-hybrid system is believed to exploit the ability of a pair of interacting proteins to bring the transcription activation domain into a more favorable position relative to the DNA binding domain such that when the DNA binding domain binds to the DNA binding site on the gene, the transactivation domain more effectively activates the promoter (see, for example, U.S. Pat. No. 5,283,173). The two-hybrid gene expression system comprises two gene expression cassettes; the first encoding a DNA binding domain fused to a nuclear receptor polypeptide, and the second encoding a transactivation domain fused to a different nuclear receptor polypeptide. In the presence of ligand, it is believed that a conformational change is induced which promotes interaction of the first polypeptide with the second polypeptide thereby resulting in dimerization of the DNA binding domain and the transactivation domain. Since the DNA binding and transactivation domains reside on two different molecules, the background activity in the absence of ligand is greatly reduced.

Another surprising discovery was that certain modifications of the two-hybrid system could also provide improved sensitivity to non-steroidal ligands for example, diacylhydrazines, when compared to steroidal ligands for example, ponasterone A (“PonA”) or muristerone A (“MurA”). That is, when compared to steroids, the non-steroidal ligands provided higher gene transcription activity at a lower ligand concentration. Furthermore, the two-hybrid system avoids some side effects due to overexpression of RXR that can occur when unmodified RXR is used as a switching partner. In a preferred two-hybrid system, native DNA binding and transactivation domains of EcR or RXR are eliminated and as a result, these hybrid molecules have less chance of interacting with other steroid hormone receptors present in the cell, thereby resulting in reduced side effects.

The ecdysone receptor (EcR) is a member of the nuclear receptor superfamily and is classified into subfamily 1, group H (referred to herein as “Group H nuclear receptors”). The members of each group share 40-60% amino acid identity in the E (ligand binding) domain (Laudet et al., A Unified Nomenclature System for the Nuclear Receptor Subfamily, 1999; Cell 97: 161-163). In addition to the ecdysone receptor, other members of this nuclear receptor subfamily 1, group H include: ubiquitous receptor (UR), Orphan receptor 1 (OR-1), steroid hormone nuclear receptor 1 (NER-1), RXR interacting protein-15 (RIP-15), liver x receptor β (LXRβ), steroid hormone receptor like protein (RLD-1), liver x receptor (LXR), liver x receptor α (LXRα), farnesoid x receptor (FXR), receptor interacting protein 14 (RIP-14), and farnesol receptor (HRR-1).

In some cases, an inducible promoter can be a small molecule ligand-inducible two polypeptide ecdysone receptor-based gene switch, such as Intrexon Corporation's RHEOSWITCH® gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/1J52002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919 each of which is incorporated by reference in its entirety.

Provided are systems for modulating the expression of a heterologous gene and an interleukin in a host cell, comprising polynucleotides expressing gene-switch polypeptides disclosed herein.

In some embodiments are systems for modulating the expression of a heterologous gene and a cytokine in a host cell, comprising a first gene expression cassette comprising a first polynucleotide encoding a first polypeptide; a second gene expression cassette comprising a second polynucleotide encoding a second polypeptide; and a ligand; wherein said first and second polypeptides comprise one or more of: (i) a transactivation domain; (ii) a DNA-binding domain; and (iii) a ligand binding domain; (iv) said heterologous gene; and (vi) said cytokine such that upon contacting said host cell with said first gene expression cassette and said second gene expression cassette in the presence of said ligand, said heterologous gene and said cytokine are expressed in said host cell. In some cases, the heterologous gene comprises an antigen binding polypeptide described herein. In some cases, the cytokine comprises at least one chemokine, interferon, interleukin, lymphokine, tumor necrosis factor, or variant or combination thereof. In some cases, the cytokine is an interleukin. In some cases the interleukin is at least one of IL12, IL2, IL15, IL21, and functional variants and fragments thereof. In some embodiments, the cytokines can be membrane bound or secreted. In other embodiments, the cytokines can be intracellular. The interleukin can comprise membrane bound IL-15 (mbIL-15) or a fusion of IL-15 and IL-15Rα. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the I-15 is indirectly linked to the IL-15Rα through a linker, In some instances, the mbIL-15 is as described in Hurton et a., “Tethered IL-15 augments antitumor activity and promotes a stem-cell memory subset in tumor-specific T cells,” Proc. Nat. Acad. Sci. USA 113(48):E7788-E7797 (2016). In another aspect, the interleukin can comprise IL-12. In some embodiments, the IL-12 is a single chain IL-12 (scIL-12), protease sensitive IL-12, destabilized IL-L12, membrane bound IL-12, intercalated IL-12. In some instances, the IL-12 variants are as described in WO 2015/095249, WO 2016/048903, WO 2017/062953, all of which is incorporated by reference in their entireties.

Provided herein are polynucleotides encoding gene switch polypeptides, wherein said gene switch polypeptides comprise: a) a first gene switch polypeptide comprising a DNA-binding domain fused to a nuclear receptor ligand binding domain, and b) a second gene switch polypeptide comprising a transactivation domain fused to a nuclear receptor ligand binding domain, wherein the first gene switch polypeptide and the second gene switch polypeptide are connected by a linker. In some cases, the linker can be a linker described herein, for instance GSG linker, furinlink, a 2A linker such as F/T2A, T2A, p2A, GSG-p2A, variants and derivatives thereof. In other instances, the linker can be an IRES.

In some cases, the DNA binding domain (DBD) comprises a DBD described herein, for instance at least one of GAL4 (GAL4 DBD), a LexA DBD, a transcription factor DBD, a steroid/thyroid hormone nuclear receptor superfamily member DBD, a bacterial LacZ DBD, and a yeast DBD. The transactivation domain can comprise a transactivation domain described herein, for instance one of a VP16 transactivation domain, a p53 transactivation domain and a B42 acidic activator transactivation domain. The Nuclear receptor ligand binding domain can comprise at least one of a ecdysone receptor (EcR), a ubiquitous receptor, an orphan receptor 1, a NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor.

In some cases, the gene switch polypeptides connected by a polypeptide linker or ribosome-skipping sequence exhibit improved dose-dependent ligand-inducible control of gene expression compared to a ligand-inducible gene switch wherein the gene switch polypeptides are connected by non-coding sequences, such as an IRES. In some cases, the gene switch polypeptides connected by a 2A linker can exhibit improved dose-dependent ligand-inducible control of heterologous gene expression compared to a gene switch wherein said gene switch polypeptides are separated by an IRES.

In some embodiments, the gene switch comprises a VP16 transactivation domain. In one embodiment, the gene switch comprises at least one of an ecdysone receptor (EcR), a ubiquitous receptor, an orphan receptor 1, a NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor. In another embodiment, a DNA-binding domain (DBD) of the gene switch comprises at least one of GAL4 (GAL4 DBD), a LexA DBD, a transcription factor DBD, a steroid/thyroid hormone nuclear receptor superfamily member DBD, a bacterial LacZ DBD, and a yeast DBD. In yet another case, the gene switch further comprises at least one of ultraspiracle protein (USP), retinoid receptor X (RXR), functional fragments and variants thereof wherein said functional fragments and variants are capable of binding to an EcR.

The polypeptides and polynucleotides as described herein can be expressed in an engineered cell. Herein an engineered cell is a cell which has been modified from its natural or endogenous state. An example of an engineered cell is a cell described herein which has been modified (e.g., by transfection of a polynucleotide into the cell) to encode for example, gene switch polypeptides, gene of interest (GOI), cell tags, heterologous genes and any other polypeptides and polynucleotides described herein.

### Ligands

In some embodiments, a ligand used for inducible gene switch regulation can be selected from any of, but without limitation to, following: N-[(1R)-1-(1,1-dimethylethyl)butyl]-N′-(2-ethyl-3-methoxybenzoyl)-3,5-dimethylbenzohydrazide (also referred to as veledimex), (2S,3R,5R,9R,10R,13R,14S,17R)-17-[(2S,3R)-3,6-dihydroxy-6-methylheptan-2-yl]-2,3,14-trihydroxy-10,13-dimethyl-2,3,4,5,9,11,12,15,16,17-decahydro-1H-cyclopenta[a]phenanthren-6-one; N′-(3,5-Dimethylbenzoyl)-N′-[(3R)-2,2-dimethyl-3-hexanyl]-2-ethyl-3-methoxybenzohydrazide; 5-Methyl-2,3-dihydro-benzo[1,4]dioxine-6-carboxylic acid N′-(3,5-dimethyl-benzoyl)-N′-(1-ethyl-2,2-dimethyl-propyl)-hydrazide; 5-Methyl-2,3-dihydro-benzo[1,4]dioxine-6-carboxylic acid N′-(3,5-dimethoxy-4-methyl-benzoyl)-N′-(1-ethyl-2,2-dimethyl-propyl)-hydrazide; 5-Methyl-2,3-dihydro-benzo[1,4]dioxine-6-carboxylic acid N′-(1-tert-butyl-butyl)-N′-(3,5-dimethyl-benzoyl)-hydrazide; 5-Methyl-2,3-dihydro-benzo[1,4]dioxine-6-carboxylic acid N′-(1-tert-butyl-butyl)-N′-(3,5-dimethoxy-4-methyl-benzoyl)-hydrazide; 5-Ethyl-2,3-dihydro-benzo[1,4]dioxine-6-carboxylic acid N′-(3,5-dimethyl-benzoyl)-N′-(1-ethyl-2,2-dimethyl-propyl)-hydrazide; 5-Ethyl-2,3-dihydro-benzo[1,4]dioxine-6-carboxylic acid N′-(3,5-dimethoxy-4-methyl-benzoyl)-N′-(1-ethyl-2,2-dimethyl-propyl)-hydrazide; 5-Ethyl-2,3-dihydro-benzo[1,4]dioxine-6-carboxylic acid N′-(1-tert-butyl-butyl)-N′-(3,5-dimethyl-benzoyl)-hydrazide; 5-Ethyl-2,3-dihydro-benzo[1,4]dioxine-6-carboxylic acid N′-(1-tert-butyl-butyl)-N′-(3,5-dimethoxy-4-methyl-benzoyl)-hydrazide; 3,5-Dimethyl-benzoic acid N-(1-ethyl-2,2-dimethyl-propyl)-N′-(3-methoxy-2-methyl-benzoyl)-hydrazide; 3,5-Dimethoxy-4-methyl-benzoic acid N-(1-ethyl-2,2-dimethyl-propyl)-N′-(3-methoxy-2-methyl-benzoyl)-hydrazide; 3,5-Dimethyl-benzoic acid N-(1-tert-butyl-butyl)-N′-(3-methoxy-2-methyl-benzoyl)-hydrazide; 3,5-Dimethoxy-4-methyl-benzoic acid N-(1-tert-butyl-butyl)-N′-(3-methoxy-2-methyl-benzoyl)-hydrazide; 3,5-Dimethyl-benzoic acid N-(1-ethyl-2,2-dimethyl-propyl)-N′-(2-ethyl-3-methoxy-benzoyl)-hydrazide; 3,5-Dimethoxy-4-methyl-benzoic acid N-(1-ethyl-2,2-dimethyl-propyl)-N′-(2-ethyl-3-methoxy-benzoyl)-hydrazide; 3,5-Dimethyl-benzoic acid N-(1-tert-butyl-butyl)-N′-(2-ethyl-3-methoxy-benzoyl)-hydrazide; 3,5-Dimethoxy-4-methyl-benzoic acid N-(1-tert-butyl-butyl)-N′-(2-ethyl-3-methoxy-benzoyl)-hydrazide; 2-Methoxy-nicotinic acid N-(1-tert-butyl-pentyl)-N′-(4-ethyl-benzoyl)-hydrazide; 3,5-Dimethyl-benzoic acid N-(2,2-dimethyl-1-phenyl-propyl)-N′-(4-ethyl-benzoyl)-hydrazide; 3,5-Dimethyl-benzoic acid N-(1-tert-butyl-pentyl)-N′-(3-methoxy-2-methyl-benzoyl)-hydrazide; and 3,5-Dimethoxy-4-methyl-benzoic acid N-(1-tert-butyl-pentyl)-N′-(3-methoxy-2-methyl-benzoyl)-hydrazide.

In some cases, a ligand used for dose-regulated control of ecdysone receptor-based inducible gene switch can be selected from any of, but without limitation to, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; arnidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present disclosure include RG-115819 (3,5-Dimethyl-benzoic acid N-(1-ethyl-2,2-dimethyl-propy1)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-buty1)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

### Cytokines

In certain embodiments, HBV vaccine antigens provided herein may be co-delivered and/or co-expressed (e.g., as part of the same HBV antigen delivery vector or via a separate vector) along with other cytokines. Provided herein are polynucleotides encoding gene-switch polypeptides and a cytokine, or variant or derivative thereof, and methods and systems incorporating the same. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily: XCL1 and XCL2; and the CX3C subfamily CX3CL1.

In certain embodiments, HBV vaccine antigens provided herein may be co-delivered and/or co-expressed (e.g., as part of the same HBV antigen delivery vector or via a separate vector) along with other interferons. Interferons (IFNs) comprise interferon type I (e.g. IFN-α, IFN-β, IFN-ε, IFN-κ, and IFN-ω), interferon type II (e.g. IFN-γ), and interferon type III. In some embodiments, IFN-α is further classified into about 13 subtypes including IFNA1, IFNA2, IFNA4, IFNA5, IFNA6, IFNA7, IFNA8, IFNA10, IFNA13, IFNA14, IFNA16, IFNA17, and IFNA21.

In certain embodiments, HBV vaccine antigens provided herein may be co-delivered and/or co-expressed (e.g., as part of the same HBV antigen delivery vector or via a separate vector) along with other interleukins. Interleukins are expressed by leukocytes or white blood cells and they promote the development and differentiation of T and B lymphocytes and hematopoietic cells. Exemplary interleukines include IL-1, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36. In some embodiments, interleukins are IL-2, IL-12, IL-15, IL-21 or a fusion of IL-15 and IL-15a.

In some aspects, the interleukin can comprise IL-12. In some embodiments, the IL-12 is a single chain IL-12 (scIL-12), protease sensitive IL-12, destabilized IL-12, membrane bound IL-12, intercalated IL-12. In some instances, the IL-12 variants are as described in WO 2015/095249, WO 2016/048903, WO 2017/062953, all of which is incorporated by reference in their entireties.

In some embodiments, an interleukin comprises mbIL-15. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the IL-15 is indirectly linked to the IL-15Rα through a linker. In some instances, the mbIL-15 is as described in Hurton et al., 2016.

In certain embodiments, HBV vaccine antigens provided herein may be co-delivered and/or co-expressed (e.g., as part of the same HBV antigen delivery vector or via a separate vector) along with other tumor necrosis factors. Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

In certain embodiments, HBV vaccine antigens provided herein may be co-delivered and/or co-expressed (e.g., as part of the same HBV antigen delivery vector or via a separate vector) along with other colony stimulating factors. Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In some embodiments, the cytokine is a membrane-bound cytokine, which is co-expressed with a chimeric antigen receptor described herein. In some embodiments, one or more methods described herein further comprise administration of a cytokine. In some instances, the cytokine comprises a chemokine, an interferon, an interleukin, a colony-stimulating factor or a tumor necrosis factor. In some instances, one or more methods described herein further comprise administration of a cytokine selected from a chemokine, an interferon, an interleukin, a colony-stimulating factor or a tumor necrosis factor. In some instances, one or more methods described herein further comprise administration of a cytokine selected from IL2, IL7, IL12, IL15, a fusion of IL-15 and IL-15Rα, IL21, IFNγ or TNF-α.

### Interleukin-12

In particular embodiments, HBV vaccine antigens provided herein may be co-delivered and/or co-expressed (e.g., as part of the same HBV antigen delivery vector or via a separate vector) along with Interleukin-12. Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as p70), and a homodimer of p40 are formed following protein synthesis. IL-12 is the master regulator of the immune system. IL-12 promotes immune response by activating NK cells and T cells (FIG. 18).

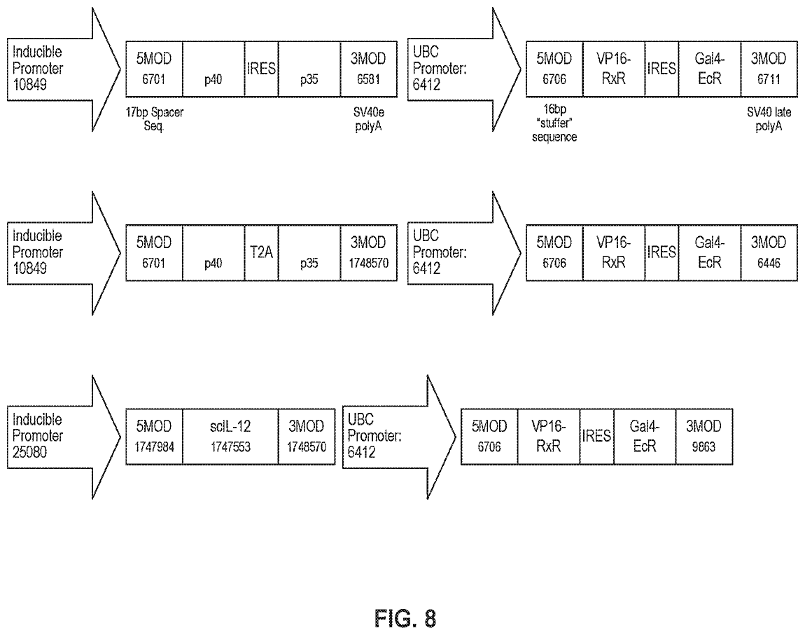

Provided herein are compositions, kits, and system comprising and methods of making HBV recombinant vaccines. The present disclosure provides HBV antigen designs (HBV designs 1-5) constructed in a multi-deleted gorilla adenovector (GC46) (SEQ ID NOS: 61-63). Also provided herein are polynucleotides encoding gene-switch polypeptides and IL-12 or variant or derivative thereof, and methods and systems incorporating the same (FIG. 8).

### Linkers

Also disclosed are constructs comprising a linker to facilitate the expression and functionality of the polynucleotides and polypeptides described herein. In some embodiments, a polynucleotide linker can be utilized in a polynucleotide described herein. A polynucleotide linker can be a double-stranded segment of DNA containing desired restriction sites that can be added to create end structures that are compatible with a vector comprising a polynucleotide described herein. In some cases, a polynucleotide linker can be useful for modifying vectors comprising polynucleotides described herein. For example, a vector modification comprising a polynucleotide linker can be a change in a multiple cloning site, or the addition of a poly-histidine tail. Polynucleotide linkers can also be used to adapt the ends of blunt insert DNA for cloning into a vector cleaved with a restriction enzyme with cohesive end termini. The use of polynucleotide linkers can be more efficient than a blunt ligation into a vector and can provide a method of releasing an insert from a vector in downstream applications. In some cases an insert can be a polynucleotide sequence encoding polypeptides useful for therapeutic applications. In some cases, a linker can be a cleavable linker.

A polynucleotide linker can be an oligomer. A polynucleotide linker can be a DNA double strand, single strand, or a combination thereof. In some cases, a linker can be RNA. A polynucleotide linker can be ligated into a vector comprising a polynucleotide described herein by a T4 ligase in some cases. To facilitate a ligation an excess of polynucleotide linkers can be added to a composition comprising an insert and a vector. In some cases, an insert and vector are pre-treated before a linker is introduced. For example, pre-treatment with a methylase can prevent unwanted cleavage of insert DNA.

In certain embodiments, two or more polypeptides encoded by a polynucleotide described herein can be separated by an intervening sequence encoding an intervening linker polypeptide. Herein the term “intervening linker polypeptide” referring to an amino acid sequence separating two or more polypeptides encoded by a polynucleotide is distinguished from the term “peptide linker” which refers to the sequence of amino acids which is optionally included in a polypeptide construct disclosed herein to connect the transmembrane domain to the cell surface polypeptide (e.g., comprising a truncated variant of a natural polypeptide). In certain cases, the intervening linker is a cleavage-susceptible intervening linker polypeptide. In some embodiments, the linker is a cleavable or ribosome skipping linker. In some embodiments, the cleavable linker or ribosome skipping linker sequence is selected from the group consisting of 2A, GSG-2A, GSG linker, SGSG linker, furinlink variants and derivatives thereof. In some embodiments, the 2A linker is a p2A linker, a T2A linker, F2A linker or E2A linker. In some embodiments, polypeptides of interest are expressed as fusion proteins linked by a cleavage-susceptible intervening linker polypeptide. In certain embodiments, cleavage-susceptible intervening linker polypeptide(s) can be any one or more of: F/T2A, T2A, p2A, 2A, GSG-p2A, GSG linker, and furinlink variants. Linkers (polynucleotide and polypeptide sequences) as disclosed in PCT/US2016/061668 (WO2017083750) published 18 May 2017 are incorporated by reference herein. In certain embodiments, the linker polypeptide comprises disclosed in the table below:

TABLE 2
Linker amino acid sequences and polynucleotide sequences

| | SEQ | | SEQ | |
|---|---|---|---|---|
| | ID | Polynucleotide Sequence | ID | Amino Acids Sequence |
| Linker Name | NO: | (5′ to 3′ where applicable) | NO: | (5′ to 3′ where applicable) |
| Whitlow Linker | 64 | GGCAGCACCTCCGGCAGCG | 81 | GSTSGSGKPGSGEGSTKG |
| | | GCAAGCCTGGCAGCGGCGA | | |
| | | GGGCAGCACCAAGGGC | | |
| Linker | 65 | TCTGGCGGAGGATCTGGAG | 82 | SGGGSGGGGSGGGGSGG |
| | | GAGGCGGATCTGGAGGAGG | | GGSGGGSLQ |
| | | AGGCAGTGGAGGCGGAGGA | | |
| | | TCTGGCGGAGGATCTCTGC | | |
| | | AG | | |
| GSG linker | 66 | GGAAGCGGA | 83 | GSG |
| SGSG linker | 67 | AGTGGCAGCGGC | 84 | SGSG |
| (G4S)3 linker | 68 | GGTGGCGGTGGCTCGGGCG | 85 | GGGGSGGGGSGGGGS |
| | | GTGGTGGGTCGGGTGGCGG | | |
| | | CGGATCT | | |
| Furin cleavage | 69 | CGTGCAAAGCGT | 86 | RAKR |
| site/Furinlink1 | | | | |
| Fmdv | 70 | AGAGCCAAGAGGGCACCGG | 87 | RAKRAPVKQTLNFDLLKL |
| | | TGAAACAGACTTTGAATTTT | | AGDVESNPGP |
| | | GACCTTCTGAAGTTGGCAG | | |
| | | GAGACGTTGAGTCCAACCC | | |
| | | TGGGCCC | | |
| Thosea asigna | 71 | GAGGGCAGAGGAAGTCTGC | 88 | EGRGSLLTCGDVEENPGP |
| virus 2A region | | TAACATGCGGTGACGTCGA | | |
| (T2A) | | GGAGAATCCTGGACCT | | |
| Furin-GSG-T2A | 72 | AGAGCTAAGAGGGGAAGCG | 89 | RAKRGSGEGRGSLLTCGD |
| | | GAGAGGGCAGAGGAAGTCT | | VEENPGP |
| | | GCTAACATGCGGTGACGTC | | |
| | | GAGGAGAATCCTGGACCT | | |
| Furin-SGSG- | 73 | AGGGCCAAGAGGAGTGGCA | 90 | RAKRSGSGEGRGSLLTCG |
| T2A | | GCGGCGAGGGCAGAGGAA | | DVEENPGP |
| | | GTCTTCTAACATGCGGTGAC | | |
| | | GTGGAGGAGAATCCCGGCC | | |
| | | CT | | |
| Porcine | 74 | GCAACGAACTTCTCTCTCCT | 91 | ATNFSLLKQAGDVEENPG |
| teschovirus-1 | | AAAACAGGCTGGTGATGTG | | P |
| 2A region | | GAGGAGAATCCTGGTCCA | | |
| (P2A) | | | | |
| GSG-P2A | 75 | GGAAGCGGAGCTACTAACT | 92 | GSGATNFSLLKQAGDVEE |
| | | TCAGCCTGCTGAAGCAGGC | | NPGP |
| | | TGGAGACGTGGAGGAGAAC | | |
| | | CCTGGACCT | | |
| Equine rhinitis | 76 | CAGTGTACTAATTATGCTCT | 93 | QCTNYALLKLAGDVESNP |
| A virus 2A | | CTTGAAATTGGCTGGAGAT | | GP |
| region (E2A) | | GTTGAGAGCAACCCTGGAC | | |
| | | CT | | |
| Foot-and-mouth | 77 | GTCAAACAGACCCTAAACT | 94 | VKQTLNFDLLKLAGDVES |
| disease virus 2A | | TTGATCTGCTAAAACTGGCC | | NPGP |
| region (F2A) | | GGGGATGTGGAAAGTAATC | | |
| | | CCGGCCCC | | |
| FP2A | 78 | CGTGCAAAGCGTGCACCGG | 95 | RAKRAPVKQGSGATNFSL |
| | | TGAAACAGGGAAGCGGAGC | | LKQAGDVEENPGP |
| | | TACTAACTTCAGCCTGCTGA | | |
| | | AGCAGGCTGGAGACGTGGA | | |
| | | GGAGAACCCTGGACCT | | |
| Linker-GSG | 79 | GCACCGGTGAAACAGGGAA | 96 | APVKQGSG |
| | | GCGGA | | |
| Linker | 80 | GCACCGGTGAAACAG | 97 | APVKQ |

In some embodiments, an intervening linker polypeptide comprises an amino acid sequence having at least 70%, 75%, 80%, 85%, 90%, 95%, 99%, 99.5% or 100% identity with the amino acid sequence of Whitlow linker (SEQ ID NO: 64), GSG linker (SEQ ID NO: 66), SGSG linker (SEQ ID NO: 67), (G4S)3 linker (SEQ ID NO: 68), Furin cleavage site/Furlink1 (SEQ ID NO: 69), Fmdv linker (SEQ ID NO: 70), Thosea asigna virus 2A region (T2A) (SEQ ID NO: 71), Furin-GSG-T2A (SEQ ID NO: 72), Furin-SGSG-T2A (SEQ ID NO: 73), porcine teschovirus-1 2A region (P2A) (SEQ ID NO: 74), GSG-P2A (SEQ ID NO: 75), equine rhinitis A virus 2A region (E2A) (SEQ ID NO: 76), or foot-and-mouth disease virus 2A region (F2A) (SEQ ID NO: 78) (Table 2). In some cases, an intervening linker polypeptide comprises an amino acid sequence having at least 70%, 75%, 80%, 85%, 90%, 95%, 99%, 99.5% or 100% identity with the amino acid sequence of linkers (SEQ ID NOS: 65, 79 80) In some cases, a viral 2A sequence can be used. 2A elements can be shorter than IRES, having from 5 to 100 base pairs. In some cases, a 2A sequence can have 5, 10, 15, 20, 25, 30, 35, 40, 45, 50, 55, 60, 65, 70, 75, 80, 85, 90, or 100 nucleotides in length. 2A linked genes can be expressed in one single open reading frame and “self-cleavage” can occur co-translationally between the last two amino acids, GP, at the C-terminus of the 2A polypeptide, giving rise to equal amounts of co-expressed proteins.

A viral 2A sequence can be about 20 amino acids. In some cases, a viral 2A sequence can contain a consensus motif Asp-Val/Ile-Glu-X-Asn-Pro-Gly-Pro. A consensus motif sequence can act co-translationally. For example, formation of a normal peptide bond between a glycine and proline residue can be prevented, which can result in ribosomal skipping and cleavage of a nascent polypeptide. This effect can produce multiple genes at equimolar levels.

A 2A peptide can allow translation of multiple proteins in a single open reading frame into a polypeptide that can be subsequently cleaved into individual polypeptide through a ribosome-skipping mechanism (Funston et al., J. Gen. Virol. 89 (Pt 2):389-96 (2008)). In some embodiments, a 2A sequence can include: F/T2A, T2A, p2A, 2A, T2A, E2A, F2A, and BmCPV2A, BmIFV2A, and any combination thereof.

In some cases, a vector can comprise an IRES sequence and a 2A linker sequence. In other cases, expression of multiple genes linked with 2A peptides can be facilitated by a spacer sequence (GSG) ahead of the 2A peptides. In some cases, constructs can combine a spacers, linkers, adaptors, promoters, or combinations thereof. For example, a linker can have a spacer (SGSG or GSG or Whitlow linker) and furin linker (R-A-K-R) cleavage site with different 2A peptides. A spacer can be an I-Ceui. In some cases, a linker can be engineered. For example, a linker can be designed to comprise chemical characteristics such as hydrophobicity. In some cases, at least two linker sequences can produce the same protein. In other cases, multiple linkers can be used in a vector. For example, genes of interest can be separated by at least two linkers.

In certain embodiments, two or more polypeptides encoded by a polynucleotide described herein can be separated by an intervening sequence encoding a linker polypeptide. In certain cases, the linker is a cleavage-susceptible linker. In some embodiments, polypeptides of interest are expressed as fusion proteins linked by a cleavage-susceptible linker polypeptide. In certain embodiments, cleavage-susceptible linker polypeptide(s) can be any one or two of: Furinlink, fmdv, p2a, GSG-p2a, and/or fp2a described below. In some cases, a linker is APVKQGSG (SEQ ID NO: 96).

In certain cases, a linker polypeptide can comprise an amino acid sequence “RAKR” (SEQ ID NO: 86). In certain cases, a Furin linker polypeptide can be encoded by a polynucleotide sequence polynucleotide sequence comprising “CGTGCAAAGCGT” (SEQ ID NO: 69) or “AGAGCTAAGAGG.” (SEQ ID NO: 130).

In some embodiments, a linker can be utilized in a polynucleotide described herein. A linker can be a flexible linker, a rigid linker, an in vivo cleavable linker, or any combination thereof. In some cases, a linker can link functional domains together (as in flexible and rigid linkers) or releasing free functional domain in vivo as in in vivo cleavable linkers.

Linkers can improve biological activity, increase expression yield, and achieving desirable pharmacokinetic profiles. A linker can also comprise hydrazone, peptide, disulfide, or thioesther.

In some cases, a linker sequence described herein can include a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n (SEQ ID NO: 85). By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers included in linker sequences described herein, can be rich in small or polar amino acids such as Gly and Ser to provide good flexibility and solubility. Flexible linkers can be suitable choices when certain movements or interactions are desired for fusion protein domains. In addition, although flexible linkers cannot have rigid structures, they can serve as a passive linker to keep a distance between functional domains. The length of flexible linkers can be adjusted to allow for proper folding or to achieve optimal biological activity of the fusion proteins.

A linker described herein can further include a rigid linker in some cases. A rigid linker can be utilized to maintain a fixed distance between domains of a polypeptide. Examples of rigid linkers can be: Alpha helix-forming linkers, Pro-rich sequence, (XP)n, X-Pro backbone, A (EAAAK)nA (n=2-5), to name a few. Rigid linkers can exhibit relatively stiff structures by adopting α-helical structures or by containing multiple Pro residues in some cases.

A linker described herein can be cleavable in some cases. In other cases a linker is not cleavable. Linkers that are not cleavable can covalently join functional domains together to act as one molecule throughout an in vivo processes or an ex vivo process. A linker can also be cleavable in vivo. A cleavable linker can be introduced to release free functional domains in vivo. A cleavable linker can be cleaved by the presence of reducing reagents, proteases, to name a few. For example, a reduction of a disulfide bond can be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein can also be carried out by proteases that can be expressed in vivo under pathological conditions (e.g. cancer or inflammation), in specific cells or tissues, or constrained within certain cellular compartments. In some cases, a cleavable linker can allow for targeted cleavage. For example, the specificity of many proteases can offer slower cleavage of a linker in constrained compartments. A cleavable linker can also comprise hydrazone, peptides, disulfide, or thioesther. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation.

In certain embodiments, an fmdv linker polypeptide comprises a sequence that can be at least about 45%, 50%, 55%, 60%, 65%, 70%, 75%, 80%, 85%, 90%, 95%, 97%, 98%, 99% or 100% identical to SEQ ID NO: 87. In certain embodiments, an fmdv linker polypeptide is one or more of the linkers encoded in a single vector linking two or more fusion proteins. In certain cases, an fmdv linker polypeptide can be encoded by a polynucleotide open reading frame (ORF) nucleic acid sequence. In some cases, an ORF encoding fmdv comprises or consists of a sequence of SEQ ID NO: 70). In certain embodiments, a polynucleotide encoding fmdv is at least 45%, 50%, 55%, 60%, 65%, 70%, 75%, 80%, 85%, 90%, 95%, 97%, 98%, 99% or 100% identical to SEQ ID NO: 70).

In certain cases, a linker polypeptide can be a “p2a” linker. In certain embodiments, a p2a polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 91). In certain embodiments, the p2a linker polypeptide can be one or more of the linkers encoded in a single vector linking two or more fusion proteins. In some cases, a p2a linker polypeptide can be encoded by a polynucleotide open reading frame (ORF) nucleic acid sequence. In certain embodiments, an ORF encoding p2a comprises or consists of the sequence of SEQ ID NO: 74). In certain cases, a polynucleotide encoding p2a can be or can be about at least 45%, 50%, 55%, 60%, 65%, 70%, 75%, 80%, 85%, 90%, 95%, 97%, 98%, 99% or 100% identical to SEQ ID NO: 74).

In some cases, a linker polypeptide can be a “GSG-p2a” linker. In certain embodiments, a GSG-p2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 92). In certain embodiments, a GSG-p2a linker polypeptide can be one or more of the linkers encoded in a single vector linking two or more fusion proteins. In some cases, a GSG-p2a linker polypeptide can be encoded by a polynucleotide open-reading frame (ORF) nucleic acid sequence. An ORF encoding GSG p2a can comprise the sequence of SEQ ID NO: 75). In some cases, a polynucleotide encoding GSG-p2a can be or can be about at least 45%, 50%, 55%, 60%, 65%, 70%, 75%, 80%, 85%, 90%, 95%, 97%, 98%, 99% or 100% identical to SEQ ID NO: 75).

A linker polypeptide can be an “fp2a” linker as provided herein. In certain embodiments, a fp2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 95). In certain cases, an fp2a linker polypeptide can be one or more of the linkers encoded in a single vector linking two or more fusion proteins. In some cases, a fp2a linker polypeptide can be encoded by a polynucleotide open reading frame (ORF) nucleic acid sequence. In certain embodiments, a polynucleotide encoding an fp2a linker can be or can be about at least 45%, 50%, 55%, 60%, 65%, 70%, 75%, 80%, 85%, 90%, 95%, 97%, 98%, 99% or 100% identical to SEQ ID NO: 78).

In some cases, a linker can be engineered. For example, a linker can be designed to comprise chemical characteristics such as hydrophobicity. In some cases, at least two linker sequences can produce the same protein. A sequence can be or can be about 45%, 50%, 55%, 60%, 65%, 70%, 75%, 80%, 85%, 90%, 95%, 96%, 97%, 98%, 99%, or 100% identical to a polypeptide sequence of SEQ ID NOS: 82, 96 or 97. In other cases, multiple linkers can be used in a vector. For example, genes of interest, and one or more gene switch polypeptide sequences described herein can be separated by at least two linkers. In some cases, genes can be separated by 2, 3, 4, 5, 6, 7, 8, 9, or up to 10 linkers.

A linker can be an engineered linker. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

In some embodiments are polynucleotides encoding a polypeptide construct comprising a furin polypeptide and a 2A polypeptide, wherein the furin polypeptide and the 2A polypeptide are connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), proline (Pro)(P), phenylalanine (Phe)(F), methionine (Met)(M), tryptophan (Trp)(W). In some cases, a polypeptide linker can also include one or more GS linker sequences, for instance (GS)n, (SG)n, (GSG)n, and (SGSG)n, wherein n can be any number from zero to fifteen.

Provided are methods of obtaining an improved expression of a polypeptide construct comprising: providing a polynucleotide encoding said polypeptide construct comprising a first functional polypeptide and a second functional polypeptide, wherein said first functional polypeptide and second functional polypeptide are connected by a linker polypeptide comprising a sequence with at least 60% identity to the sequence APVKQ; and expressing said polynucleotide in a host cell, wherein said expressing results in an improved expression of the polypeptide construct as compared to a corresponding polypeptide construct that does not have a linker polypeptide comprising a sequence with at least 60% identity to the sequence APVKQ.

### IRES Elements

Also disclosed herein are constructs comprising an IRES element to facilitate the expression and functionality of the polynucleotides and polypeptides described herein. The term “internal ribosome entry site (IRES)” as used herein can be intended to mean internal ribosomal entry site. In a vector comprising an IRES sequence, a first gene can be translated by a cap-dependent, ribosome scanning, mechanism with its own 5′-UTR, whereas translation of a subsequent gene can be accomplished by direct recruitment of a ribosome to an IRES in a cap-independent manner. An IRES sequence can allow eukaryotic ribosomes to bind and begin translation without binding to a 5′ capped end. An IRES sequence can allow expression of multiple genes from one transcript (Mountford and Smith 1995).

The term “CAP” or “cap” as used herein refers to a modified nucleotide, generally a 7-methyl guanosine, linked 3′ to 5′ (7meG-ppp-G), to the 5′ end of a eukaryotic mRNA, that serves as a required element in the normal translation initiation pathway during expression of protein from that mRNA.

In certain cases, an IRES region can be derived from a virus, such as picornavirus, encephalomyocarditis virus, hepatitis C virus IRES sequence. In other cases, an IRES sequence can be derived from an encephalomyocarditis virus. The term “EMCV” or “encephalomyocarditis virus” as used herein refers to any member isolate or strain of the encephalomyocarditis virus species of the genus of the family Picornaviridae. Examples are: EMCV-R (Rueckert) strain virus, Columbia-SK virus. In some cases, a cellular IRES element, such as eukaryotic initiation factor 4G, immunoglobulin heavy chain binding protein, c-myc proto-oncogene, vascular endothelial growth factor, fibroblast growth factor-1 IRES, or any combination or modification thereof can be used. In some cases, a cellular IRES can have increased gene expression when compared to a viral IRES.

An IRES sequence of viral, cellular or a combination thereof can be utilized in a vector. An IRES can be from encephalomyocarditis (EMCV) or poliovirus (PV). In some cases, an IRES element is selected from a group consisting of Poliovirus (PV), Encephalomyelitis virus (EMCV), Foot-and-mouth disease virus (FMDV), Porcine teschovirus-1 (PTV-1), Aichivirus (AiV), Seneca Valley virus (SVV), Hepatitis C virus (HCV), Classical swine fever virus (CSFV), Human immunodeficiency virus-2 (HIV-2), Human immunodeficiency virus-1 (HIV-1), Moloney murine leukemia virus (MoMLV), Feline immunodeficiency virus (FIV), Mouse mammary tumor virus (MMTV), Human cytomegalovirus latency (pUL138), Epstein-Barr virus (EBNA-1), Herpes virus Marek's disease (MDV RLORF9), SV40 polycistronic 19S (SV40 19S), Rhopalosiphum padi virus (RhPV), Cricket paralysis virus (CrPV), Ectropis obliqua picorna-like virus (EoPV), Plautia stali intestine virus (PSIV), Triatoma virus (TrV), Bee paralysis dicistrovirus (IAPV, KBV), Black currant reversion virus (BRV), Pelargonium flower break virus (PFBV), Hibiscus chlorotic ringspot virus (HCRSV), Crucifer-infecting tobamovirus (CrTMV), Potato leaf roll polerovirus (PLRV), Tobacco etch virus (TEV), Giardiavirus (GLV), Leishmania RNA virus-1 (LRV-1), and combinations or modifications thereof. In some cases, an IRES is selected from a group consisting of Apaf-1, XIAP, HIAP2/c-IAP1, DAP5, Bcl-2, c-myc, CAT-1, INR, Differentiation LEF-1, PDGF2, HIF-1a, VEGF, FGF2, BiP, BAG-1, CIRP, p53, SHMT1, PITSLREp58, CDK1, Rpr, hid, hsp70, grim, skl, Antennapedia, dFoxO, dInR, Adh-Adhr, HSP101, ADH, URE-2, GPR1, NCE102, YMR181a, MSN1, BOIl, FLO8, GIC1, and any combination or modification thereof. When an IRES element is included between two open reading frames (ORFs), initiation of translation can occur by a canonical 5′-m7GpppN cap-dependent mechanism in a first ORF and a cap-independent mechanism in a second ORF downstream of the IRES element.

In some cases, genes can be linked by an internal ribosomal entry site (IRES). An IRES can allow simultaneous expression of multiple genes. For example, an IRES sequence can permit production of multiple proteins from a single mRNA transcript. A ribosome can bind to an IRES in a 5′-cap independent manner and initiate translation.

In some cases, an IRES sequence can be or can be about 500 base pairs. An IRES sequence can be from 300 base pairs to 1000 base pairs. For example, an IRES can be 300, 350, 400, 450, 500, 550, 600, 650, 700, 750, 800, 850, 900, 950, or 1000 base pairs long.

In some cases, expression of a downstream gene within a vector comprising an IRES sequence can be reduced. For example, a gene following an IRES sequence can have reduced expression over a gene preceding an IRES sequence. Reduced expression can be from 1% to 99% reduction over a preceding gene.

### Methods of Regulating Expression

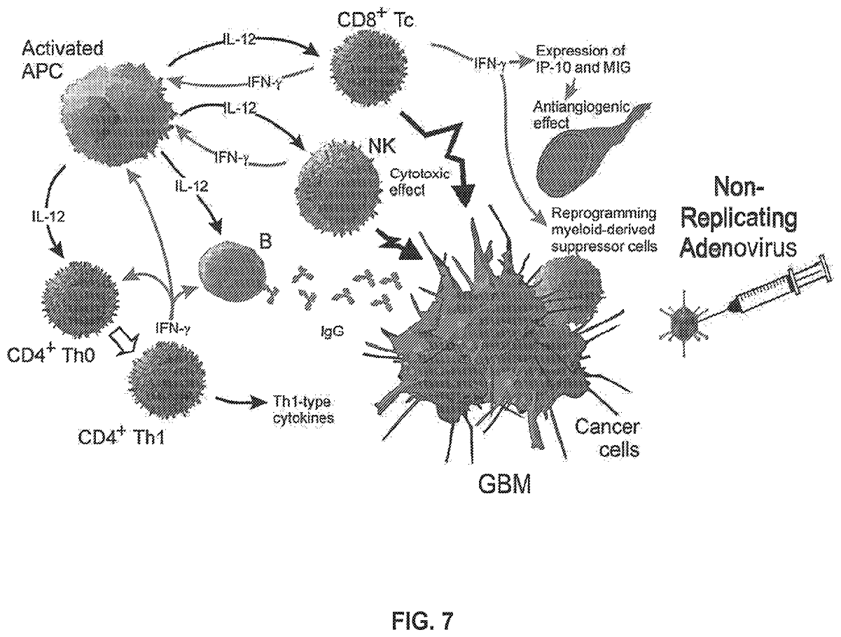

In one embodiment, a method of regulating the expression of a heterologous gene in an engineered cell is provided. Polynucleotides encoding for gene switch polypeptides for ligand inducible control of a heterologous gene expression, an antigen binding polypeptide and a heterologous gene is provided. In some instances, the polynucleotides are in one or more gene expression cassettes as depicted in any one of FIGS. 1 through 16. In another instance, the polynucleotides are incorporated into an engineered cell via viral or non-viral vectors. Viral vectors can include lentiviral vectors, retroviral vectors or adenoviral vectors. Non-viral vectors can include Sleeping Beauty transposons. In other instances, the polynucleotides are incorporated into an engineered cell via recombinases or gene editing techniques. Examples of recombinases are serine recombinases as described herein. Examples of gene editing techniques can include CRISPR or Argonaute systems. Herein a “CRISPR gene editing system” of “CRISPR system” refers to any RNA-guided Cas protein-mediated process for targeting a change in DNA sequence to a specific region of a genome. Herein “Argonaute gene editing system” refers to any single-stranded DNA guided Argonaute endonuclease-mediated process for targeting a change in DNA sequence to a specific region of a genome.

### Pharmaceutical Compositions and Dosage

The present disclosure provides a composition comprising the adenovirus or adenoviral vector described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the adenovirus or adenoviral vector. Any suitable carrier can be used within the context of the present disclosure, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the adenovirus or adenoviral vector is part of a composition formulated to protect the adenovirus or adenoviral vector from damage prior to administration. For example, the composition can be formulated to reduce loss of the adenovirus or adenoviral vector on devices used to prepare, store, or administer the adenovirus or adenoviral vector, such as glassware, syringes, or needles. The composition can be formulated to decrease the light sensitivity and/or temperature sensitivity of the adenovirus or adenoviral vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the adenovirus or adenoviral vector, and facilitate its administration. Formulations for adenovirus or adenoviral vector-containing compositions are further described in, for example, U.S. Pat. Nos. 6,225,289, 6,514,943, and International Patent Application Publication WO 2000/034444.

The composition also can be formulated to enhance transduction efficiency. In addition, one of ordinary skill in the art will appreciate that the adenovirus or adenoviral vector can be present in a composition with other therapeutic or biologically-active agents. For example, factors that control inflammation, such as ibuprofen or steroids, can be part of the composition to reduce swelling and inflammation associated with in vivo administration of the adenovirus or adenoviral vector. If the adenovirus or adenoviral vector is used to deliver an antigen-encoding nucleic acid sequence to a host, immune system stimulators or adjuvants, e.g., interleukins, lipopolysaccharide, or double-stranded RNA, can be administered to enhance or modify any immune response to the antigen. Antibiotics, i.e., microbicides and fungicides, can be present to treat existing infection and/or reduce the risk of future infection, such as infection associated with gene transfer procedures.

In some embodiments, disclosed herein are compositions comprising a polynucleotide or polypeptide disclosed herein for administration in a subject. In some instances, are modified effector cell compositions encoding a polynucleotide or polypeptide disclosed herein, and optionally containing a cytokine and/or an additional therapeutic agent. In some instances, also included herein are vectors encoding gene-switch polypeptides for regulating expression of a chimeric antigen receptor for modification of an effector cell.

In some instances, pharmaceutical compositions of a modified effector cell or a vector encoding gene-switch polypeptides and a chimeric antigen receptor are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein can also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein can benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and An anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” can include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants can be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

These examples are provided for illustrative purposes only and not to limit the scope of the claims provided herein.

### Example 1

### Antigenicity Bioinformatics Workflow for HBV Vaccine Designs

The HBV vaccine antigen designs provided herein were inspired via inventor-selected combinatorial guidance provided via use of bioinformatics analysis and in silico protein engineering methods. HBV antigen sequences were selected based on genotype D protein sequences, antigenicity predictions and T cell epitope mapping with broad coverages, which could lead to MHC-I binding and cytokine production following T cell activation. The overall workflow of the HBV vaccine designs provided herein is shown in FIG. 5 and is further detailed below.

### Obtaining Consensus Sequences

The HBV genome encodes several overlapping viral proteins, including the polymerase, core, envelope (Pre-S1, S2, S), HBe, and HBx proteins (FIGS. 3A-3D). Consensus AA sequences were obtained from the Hepatitis B Virus database (HBVdb) for A, C, and D subtypes.

### Predicting Binding Affinity

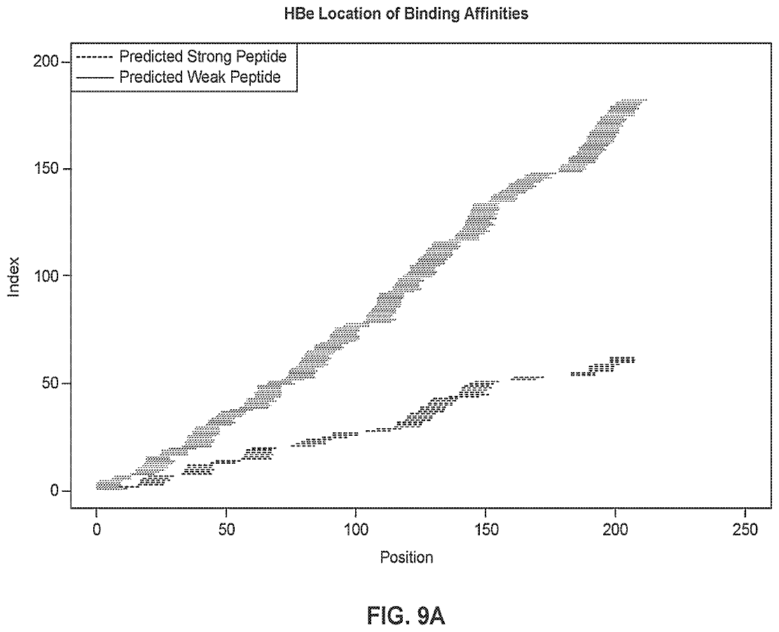

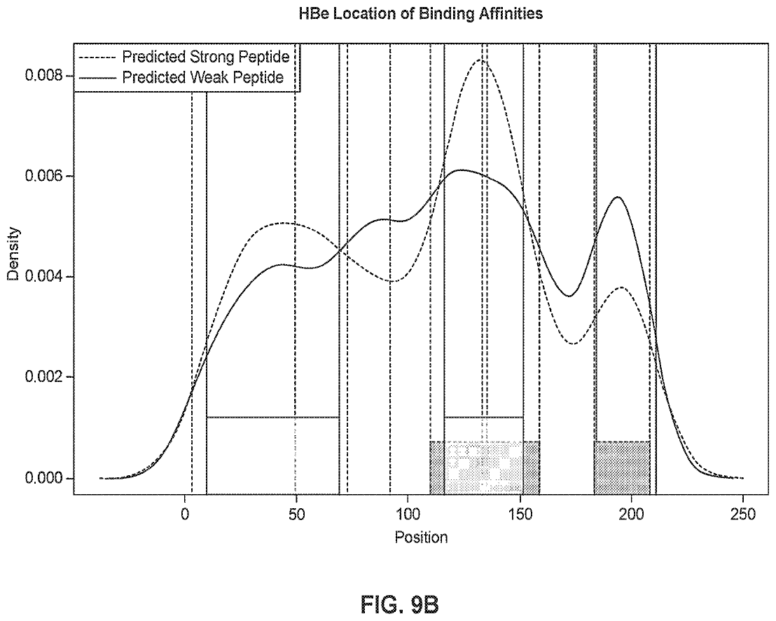

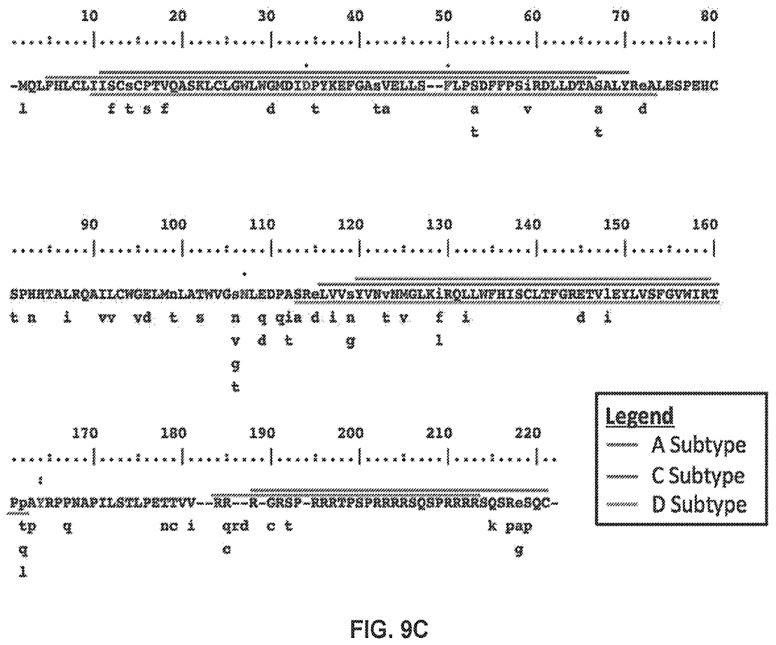

NetMHC 4.0 was applied to each consensus sequence to predict binding affinity against all major MHC-I alleles (HLA-A0101, HLA-A0201, HLA-A0301, HLA-A2402, HLA-A2601, HLA-B0702, HLA-B0801, HLA-B2705, HLA-B3901, HLA-B4001, HLA-B5801, and HLA-B1501). NetMHC 4.0 uses artificial neural networks to predict the binding affinity of peptide sequences. This analysis was performed across HBV genotypes A, C, and D. Thresholds were arbitrarily established at 0.5% (strong binders) and 2% (weak binders) ranks. Peptides with predicted binding affinity greater than 99.5% were classified as strong binders and peptides with predicted binding affinity greater than 98% were classified as weak binders. The position of each AA within the peptide sequences were extracted and used to generate density curves (FIG. 9A). Using these density curves, first and second order differentials were calculated to determine peaks for strong and weak binders (FIG. 9B). Finally, the union of these positions was used to extract AA sequences likely to elicit a response (FIG. 9C).

### Example 2

### HBV Molecular Vaccine Designs

The HBV vaccines described herein include the following HBV designs 1-5 engineered proteins (peptides with genetic modifications). Once the designs were finalized the entire sequence for each design was subjected to NetMHC prediction to assess antigenicity and coverage against A, C, and D genotypes (Example 1). The vaccines described herein were compared against TG1050, an adenovirus-based immunotherapeutic HBV vaccine currently in clinical trials, that encodes a unique large fusion protein composed of: (a) a truncated core, (b) a modified polymerase, and (c) two envelope domains. The core region of TG1050 lacked the pre-core and its polymerase was split into three segments along with four point mutations to improve the vaccine construct stability (Poll, Plo2 and Pol3; ΔAA 538-544 and ΔAA 710-742 and mutations D689H, V769Y, T776Y, D777H). The two selected envelope domains were inserted in between those polymerase segments as shown in FIG. 5. TG1050 polymerase, Env1 and Env2 sequences were obtained from Genbank (Y07587.1), and the TG1050 core sequences were obtained from HBV DB (AB048701).

Five antigen designs (HBV designs 1-5) and a control antigen were synthesized and cloned into expression plasmids—pAdShuttles for adenovector construction (FIG. 6). Initial antigen screening evaluated in vitro antigen expression in transient transfection, in vitro antigen processing and presentation in transient transfection studies of monocyte-derived dendritic cells. As shown in FIG. 17, HBV designs 1 and 2 were designed based on clade D consensus. 32 HBV peptides from Core (8), Surface (8), Polymerase (8), HBx (6) and HBPS (2) were curated from literature that have experimental and functional data, such as immunogenicity data, Mass-spec. etc. (Table 3). For HBV design 3, human ankyrin-like repeat (ALR) protein scaffold (PDB code 1QYM) peptides were grafted at the helical and loop regions in a tandem manner. Two ALR scaffolds were used, connected by a cleavable linker (VSQTSKLTR; SEQ ID NO:111). ALR proteins have generally high expression and high stability. Thus, ALR proteins were used as a scaffold for the HBV peptides to create novel CTLs. HBV design 4 epitopes were separated by KK linkers (FIG. 6).

### HBV Design 1

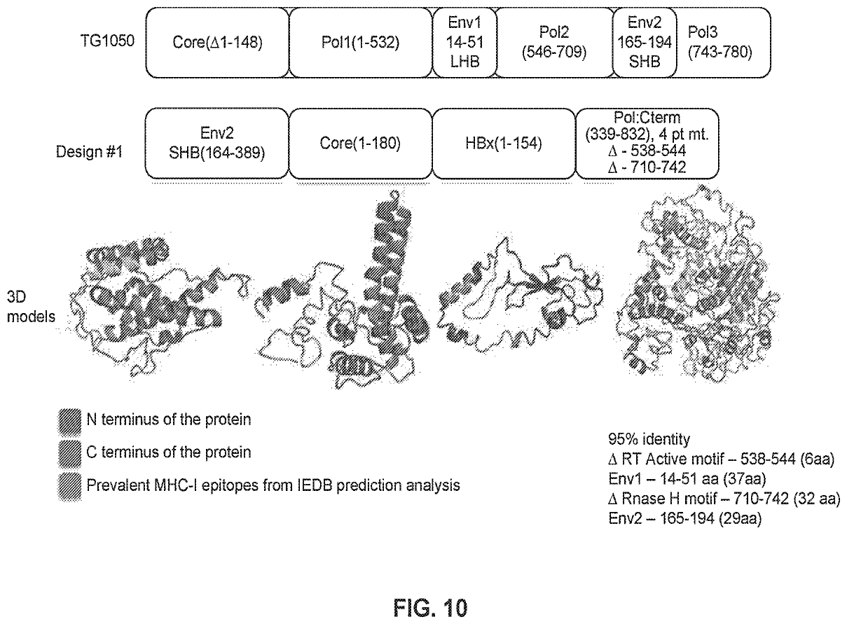

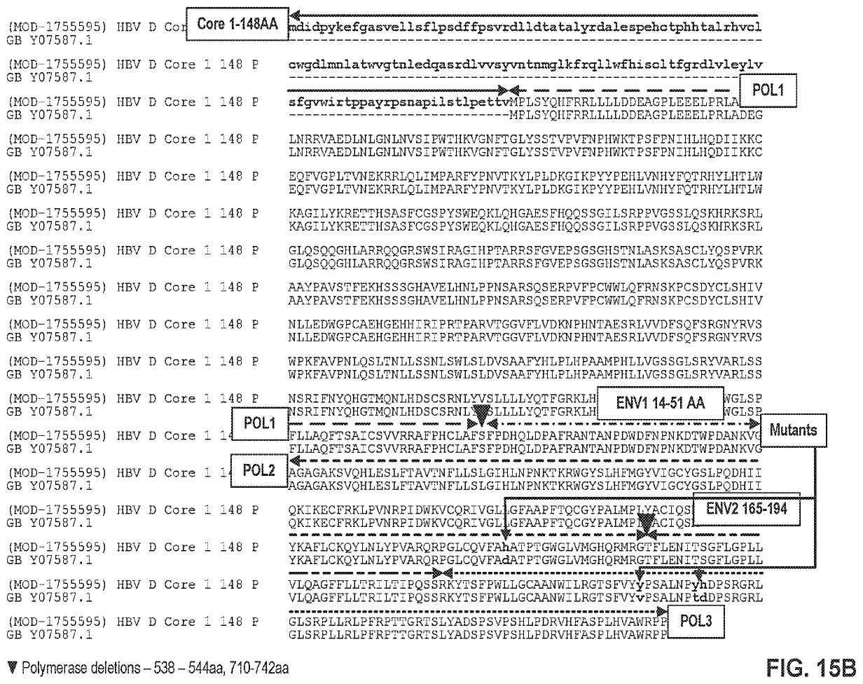

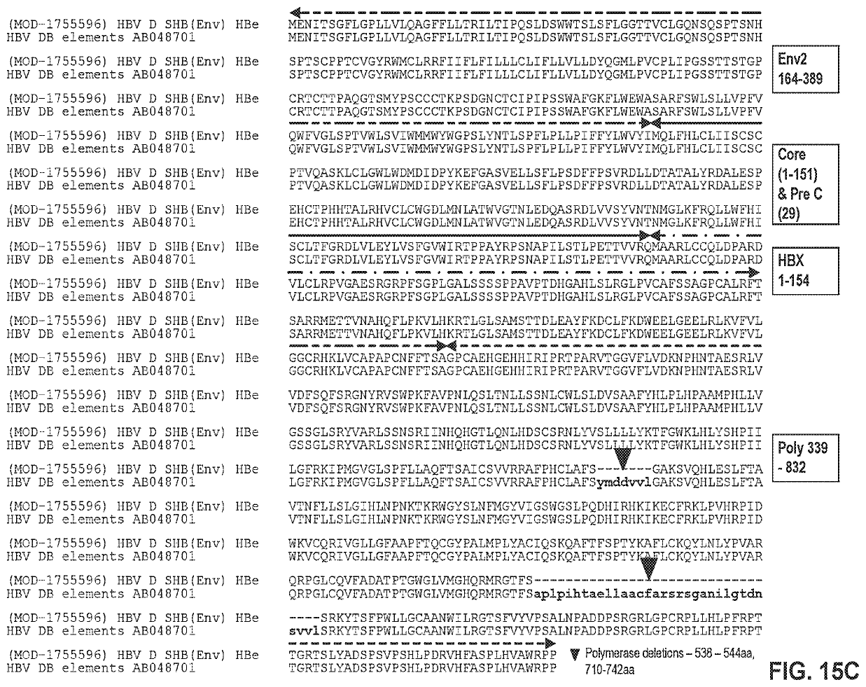

HBV D genotype protein sequence (HBV DB: AB048701) was used in this design. Antigenicity prediction analysis (Example 1) and homology models of HBV proteins guided this Design. Comparison of HBV Design 1 and TG1050 along with homology models of protein antigens is shown in FIG. 10, and sequence comparisons are shown in FIGS. 15A-15C. This design contained four native HBV antigens: (1) extended Env2/S region 165-382 AA; (2) core region 1-151 AA and pre-core region; (3) HBx region 1-154 AA; and (4) Polymerase (339-832 AA), del 538-544, 710-742 AA. The TG1050 comparator had two HBV antigens with a third envelope peptide fused to polymerase.

This design encompassed entire protein domains from envelope, core, HBx and the reverse transcriptase domain of polymerase. The TG1050 comparator had truncated core and envelope peptides fused into the modified polymerase domain, but did not encode any HBx sequences.

Different HBV domains were seamlessly fused together as a one long open reading frame. The TG1050 comparator had only two domains (core and polymerase) with truncations as well as deletions. The TG1050 comparator had random insertion of envelope peptides within the polymerase domain.

Truncations were made in the envelope protein and polymerase domains. Only the C terminus region of the envelope protein was used in this design. Only the C terminus region of reverse transcriptase was included. The entire RNaseH domain was included. Truncations were made to inactivate the reverse transcriptase function in ΔRT active motif (538-544: 6aa) and in ΔRNase H motif (710-742: 32 aa).

HBV design 1 novelty: Overall, HBV design 1 had an extended Env2/S region along with both pre-core and core regions compared to TG1050. Unlike TG1050, it also contained the HBx domain. The N-term truncated polymerase along with deletions similar to TG1050 was also used.

### HBV Design 2

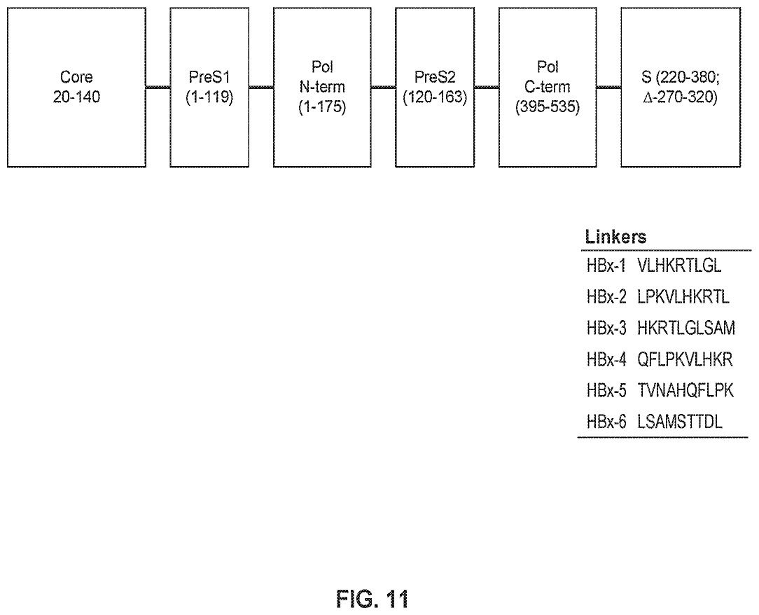

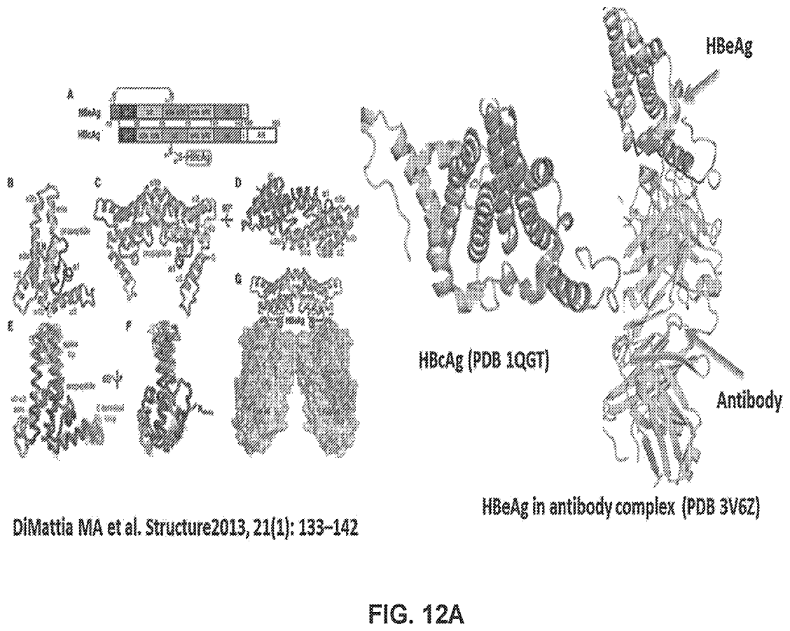

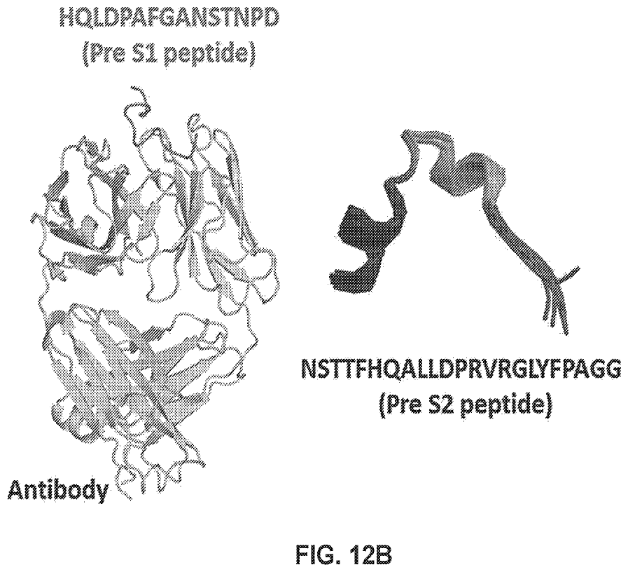

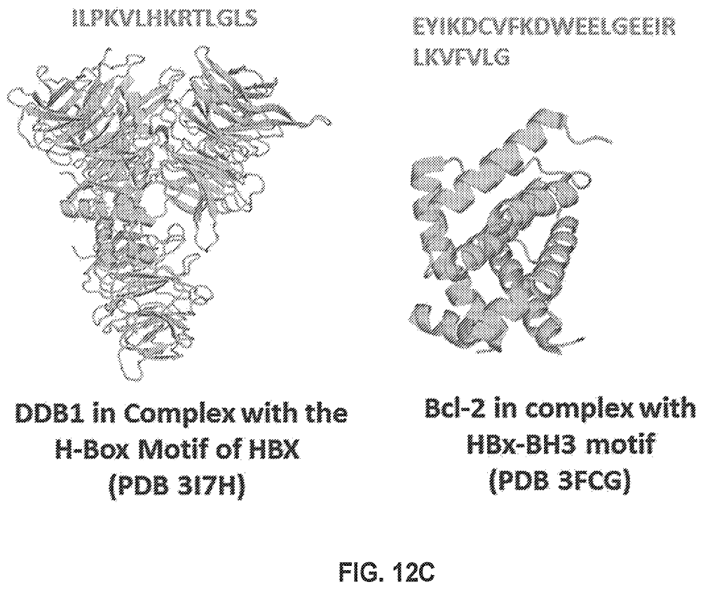

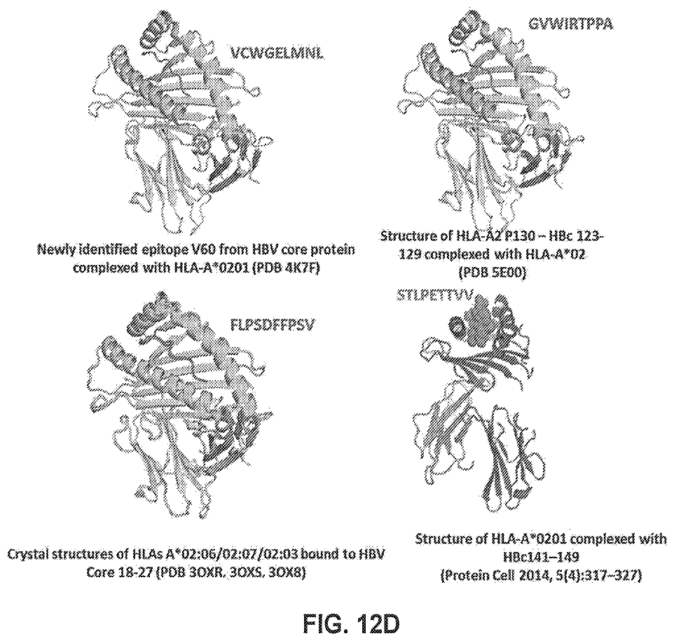

This design consisted of all three major proteins including Core, Surface splice variants, and Polymerase that were linked with HBx peptides as depicted in FIG. 11. Selected protein components were fused together to make a one long open reading frame. This design was based on the following: (a) functional genomics data (HBVdb: AB048701); (b) T cell epitope predictions (Example 1); and (c) analysis of available HBV protein structures as illustrated in FIGS. 12A-12D. When compared to the TG1050 comparator and HBV design design 1, this design has additional and/or modified protein domains as follows: (a) truncated C terminus; (b) spliced variants of surface proteins (PreS1 and PreS2, S); (c) split Pol N- and C-terminus fragments; and (d) six HBx peptides. The protein domains were shuffled to avoid any HBV infectivity, and HBV design 2 used a different ordering scheme than the TG1050 comparator and HBV design 1.

### HBV Design 3

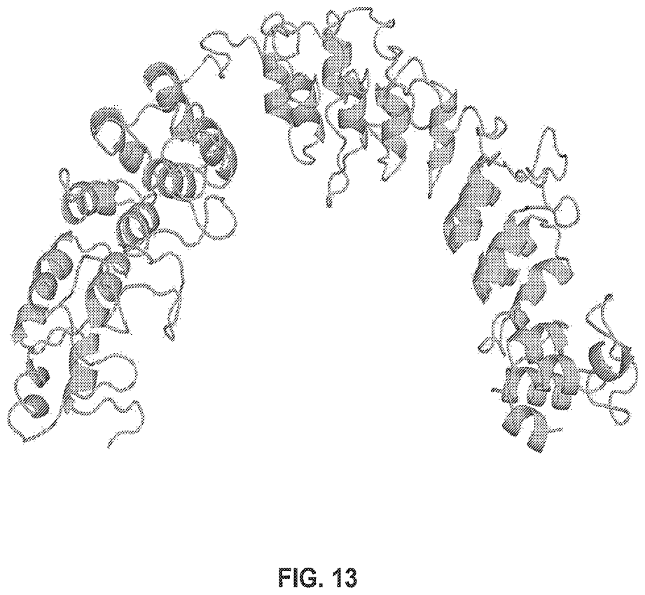

This design was based on multi-epitopes containing a total of 32 CTL specific epitopes (Table 3) and was selected from the following: (1) literature review based on multiple immunological assays and immunoproteomics; and (2) the IEDB; (3) NetMHC 4.0 epitope predictions. Human Ankyrin-like repeat protein (PDB code 1QYM) was selected as a scaffold on to which the 32 peptides were grafted replacing the repeat regions. A homology model of the this design (FIG. 13) was generated, which suggested that ankyrin scaffold with grafted peptides can retain the overall tertiary structure. Scaffold backbone amino acids connecting the 32 grafted CTL epitopes can serve as adequate linkers.

### HBV Design 4

This design was comprised of the same 32 CTL epitopes from Core, Surface, Pol, HBx and HBSP as Design 3 (Table 3). The peptides were assembled with charged dipeptide KK residue linkers instead of grafting onto a scaffold.

TABLE 3
32 HBV Peptides from Core, Surface, Polymerase, HBx, and HBPS

| Source | Sequence | SEQ | Length | Source | Sequence | SEQ | Length |
|---|---|---|---|---|---|---|---|
| Core-1 | FLPSDFFPSV | #45 | 10 | Surface-1 | GGPNLDNIL | #112 | 9 |
| Core-2 | CWGELMTL | #46 | 8 | Surface-2 | LTTVPAASLLA | #113 | 11 |
| Core-3 | GVWIRTPPA | #47 | 9 | Surface-3 | ILRSFIPLL | #114 | 9 |
| Core-4 | STLPETTVVRR | #48 | 11 | Surface-4 | FLGGPPVCL | #115 | 9 |
| Core-5 | LTFGRETVLEY | #49 | 11 | Surface-5 | FLLTRILTI | #116 | 9 |
| Core-6 | DLLDTASALY | #50 | 10 | Surface-6 | WLSLLVPFV | #117 | 9 |
| Core-7 | LWFHISCLTF | #51 | 10 | Surface-7 | GLSPTVWLSV | #118 | 10 |
| Core-8 | EYLVSFGVW | #52 | 9 | Surface-8 | LLVPFVQWFV | #119 | 10 |
| Pol-1 | FLKQQYMNL | #53 | 9 | HBSP-1 | LLLKATLCI | #120 | 9 |
| Pol-2 | FLSKQYMDL | #54 | 9 | HBSP-2 | TLCIPHVAV | #121 | 9 |
| Pol-3 | TVSTKLCKI | #55 | 9 | HBx-1 | VLHKRTLGL | #122 | 9 |
| Pol-4 | GLSRYVARL | #56 | 9 | HBx-2 | LPKVLHKRTL | #123 | 10 |
| Pol-5 | KLHLYSHPI | #57 | 9 | HBx-3 | HKRTLGLSAM | #124 | 10 |
| Pol-6 | FLLSLGIHL | #58 | 9 | HBx-4 | QFLPKVLHKR | #25 | 10 |
| Pol-7 | SLYADSPSV | #59 | 9 | HBx-5 | TVNAHQFLPK | #126 | 10 |
| Pol-8 | ALMPLYACI | #60 | 9 | HBx-6 | LSAMSTTDL | #127 | 9 |

### HBVO Design 5

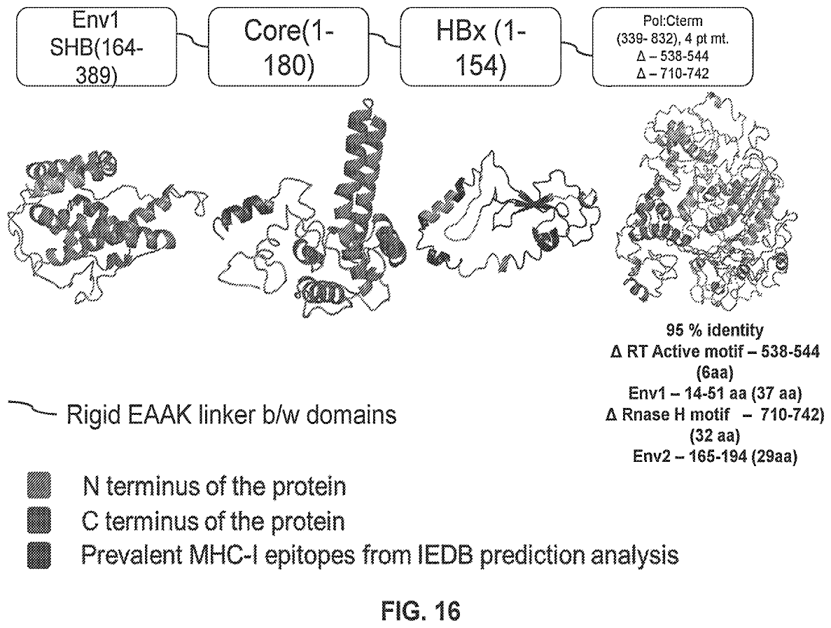

This design is similar to design 1 and has rigid linker (EAAAK)2 (SEQ ID NO: 129) between the fusion components (SEQ ID NOs: 112 and 113; Table 6; FIG. 16).

For RNA qPCR relative expression assay, 5′-TGCCAAGAGTGACGTGTCCA-3′ (SEQ ID NO: 110) was used as a splice primer, and 5′-CCCAGGTCCAACTGCAGCCGG-3′ (SEQ TD NO: 128) was used as a splice probe. Specific primers designed for each antigen were used as reverse primers (FIG. 14). A549 cells were lipotransfected (Table 4), and HT1080 cells were nucleofected (Table 5).

TABLE 4
qPCR Expression Assay (A549 Cells)

| | One-step qPCR 0.1 ng total RNA | | | | Two-step qPCR 0.2 ng cDNA | | | |
|---|---|---|---|---|---|---|---|---|
| | | | | Fold | | | | Fold |
| | | | | difference | | | | difference |
| | Short | | | in GOI | long | | | in GOI |
| | Amplicon | GOI | HPRT1 | relative to | Amplicon | GOI | HPRT1 | relative to |
| Sample | bp | Ave. Ct | Ave. Ct | untreated | bp | Ave. Ct | Ave. Ct | untreated |
| Mock | | 40.0 | 31.1 | 1 | | 40.0 | 31.3 | 1 |
| 4825853 | 141 | 23.1 | 31.3 | 145618 | 993 | 32.0 | 31.6 | 300 |
| Mock | | 40.0 | 31.1 | 1 | | 40.0 | 31.3 | 1 |
| 4825811 | 162 | 23.2 | 31.4 | 138449 | 948 | 31.2 | 31.6 | 531 |
| Mock | | 40.0 | 31.1 | 1 | | 40.0 | 31.3 | 1 |
| 4825790 | 125 | 22.5 | 31.5 | 241891 | 839 | 29.0 | 31.3 | 2016 |
| Mock | | 40.0 | 31.1 | 1 | | 40.0 | 31.3 | 1 |
| 4825791 | 127 | 22.3 | 31.6 | 304806 | 895 | 28.1 | 31.4 | 3882 |
| Mock | | 40.0 | 31.1 | 1 | | 40.0 | 31.3 | 1 |
| 4825852 | 143 | 20.8 | 31.6 | 827615 | 777 | 24.5 | 31.7 | 58169 |

TABLE 5
qPCR Expression Assay (HT1080 Cells)

| | One-step qPCR 5 ng total RNA | | | | Two-step qPCR 5 ng cDNA | | | |
|---|---|---|---|---|---|---|---|---|
| | | | | Fold | | | | Fold |
| | | | | difference | | | | difference |
| | Short | | | in GOI | long | | | in GOI |
| | Amplicon | GOI | HPRT1 | relative to | Amplicon | GOI | HPRT1 | relative to |
| Sample | bp | Ave. Ct | Ave. Ct | untreated | bp | Ave. Ct | Ave. Ct | untreated |
| Mock | | 40.0 | 25.5 | 1 | | 40.0 | 25.5 | 1 |
| 4825853 | 141 | 26.5 | 25.7 | 13981 | 993 | 32.4 | 25.1 | 143 |
| Mock | | 40.0 | 25.5 | 1 | | 40.0 | 25.5 | 1 |
| 4825811 | 162 | 23.9 | 25.5 | 71179 | 948 | 27.5 | 25.5 | 5564 |
| Mock | | 40.0 | 25.5 | 1 | | 40.0 | 25.5 | 1 |
| 4825790 | 125 | 25.5 | 25.6 | 24556 | 839 | 29.6 | 25.2 | 1114 |
| Mock | | 40.0 | 25.5 | 1 | | 40.0 | 25.5 | 1 |
| 4825791 | 127 | 24.4 | 26.1 | 78118 | 895 | 26.0 | 25.4 | 15042 |
| Mock | | 40.0 | 25.5 | 1 | | 40.0 | 25.5 | 1 |
| 4825852 | 143 | 21.8 | 25.7 | 360074 | 777 | 22.9 | 25.2 | 113697 |

### Example 3

### Immunogenicity Testing of HBV Vaccine

All of the antigens were constructed in multi-deleted gorilla adenovectors and produced to generate research materials for experimentation as well as pre-GMP stocks that can move to GMP manufacture for clinical studies (FIG. 6).

Optimum plasmid concentration for dendritic cell transfection is determined. The ability of different antigen designs to activate T cells from chronic PHBV patients is assessed. The immunogenicity of antigen design on HBV infected patient samples is assessed. The in vivo testing plan is to conduct in vivo immunogenicity studies in mice (e.g., single administration studies, repeat administration studies, including a “boost at home” proof of feasibility, route of administration (IM vs SC)), and mouse model of chronic HBV infection and eventually HCC.

Monocyte-derived dendritic cells are generated with GM-CSF and IL-4 according to standard protocol. The monocyte-derived dendritic cells are infected with different HBV antigen constructs and incubated with the vectors for 24 hours at 37° C. The cells are washed and matured overnight with 10 ng/ml rhuTNF-α. The cell maturation is assessed by flow cytometry (CD40, CD80, CD83, CD86, HLA-DR). Cytokine production is assessed for IL-1β, TNF-α, IL-6, IL-12p40, and MCP1. The monocyte-derived dendritic cells are co-cultured with autologous T cells for 10 days 1:10 ratio. Exogenous rhuIL-2 (200 U/ml), rhuIL-7 and rHuIL-15 (10 ng/ml) are added on day 3, 5 and 7. Cytokine secretions for IFN-γ, TNF-α, CXCL-10, CXCL-9, IL-10, IL-6, IL-4, IL-5, IL-12p70, and MCP1 are assessed. Activation markers for T cells are CD25, CD69, CD45RA, CD45RO, PD1, CTLA-4, TIGIT, and LAG3. Antigen design with the most immunogenicity based on dendritic cells and T cell activation is selected, and the immunogenicity of the antigen design on HBV infected patient samples is assessed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The present application claims benefit to U.S. Provisional Application No. 62/639,354 filed Mar. 6, 2018, which is incorporated herein by reference in its entirety.

### Embodiments

E1. A non-naturally occurring polynucleotide encoding a polypeptide comprising at least one or more immune response-inducing hepatitis B virus (HBV) polypeptides.

E2. The polynucleotide of E1, wherein said one or more HBV polypeptides comprises an HBV Core peptide.

E3. The polynucleotide of E2, wherein said HBV Core peptide has any one of the Core peptide sequences as shown in Table 3.

E4. The polynucleotide of any one of E1 to E3, wherein said one or more HBV polypeptides comprises an HBV Surface peptide.

E5. The polynucleotide of E4, wherein said HBV Surface peptide has any one of the Surface peptide sequences as shown in Table 3.

E6. The polynucleotide of any one of E1 to E5, wherein said one or more HBV polypeptides comprises an HBV Pol peptide.

E7. The polynucleotide of E6, wherein said HBV Pol peptide has any one of the Pol peptide sequences as shown in Table 3.

E8. The polynucleotide of any one of E1 to E7, wherein said one or more HBV polypeptides comprises an HBV HBSP/HBx peptide.

E9. The polynucleotide of E8, wherein said HBV HBSP/HBx peptide has any one of the HBSP/HBx peptide sequences as shown in Table 3.

E10. The polynucleotide of any one of E1 to E9, wherein said one or more HBV polypeptides comprises a KK linker.

E11. The polynucleotide of E10, wherein said KK linker connects any one of the peptide sequences as shown in Table 3 to any other peptide sequences as shown in Table 3.

E12. A polynucleotide comprising the polynucleotide of any one of E1 to E11, further comprising one or more polynucleotides encoding a gene switch system for inducible control of heterologous gene expression, wherein heterologous gene expression is regulated by said gene switch system; and, wherein said heterologous gene comprises the polynucleotide of any one of E1 to E11.

E13. The polynucleotide of any one of E1 to E12, wherein said gene switch system is an ecdysone receptor-based (EcR-based) gene switch system.

E14. The polynucleotide of any one of E1 to E13, wherein said one or more HBV polypeptides is for use in a vaccine.

E15. A vector comprising the polynucleotide of any one of E1 to E14.

E16. The vector of E15, wherein said vector is an adenoviral vector.

E17. The vector of E16, wherein said adenoviral vector is a gorilla adenoviral vector.

E18. A method of regulating the expression of a heterologous gene in a cell, the method comprising: introducing into said cell one or more polynucleotides that comprise (i) an repressible or inducible gene switch, and (ii) a heterologous immune response-inducing gene, wherein expression of said heterologous immune response-inducing gene is regulated by said gene switch, wherein said heterologous immune response-inducing gene encodes at least one of one or more HBV polypeptides; and exposing said cell to a compound in an amount sufficient to repress or induce expression of said heterologous immune response-inducing gene.

E19. The method of E18, wherein said target cell is a mammalian cell.

E20. The method of E18 or E19, wherein said gene switch comprises a ligand binding domain derived from at least one of an ecdysone receptor (EcR), a ubiquitous receptor, an orphan receptor 1, an NER-1, a steroid hormone nuclear receptor 1, a retinoid X receptor interacting protein-15, a liver X receptor β, a steroid hormone receptor like protein, a liver X receptor, a liver X receptor α, a farnesoid X receptor, a receptor interacting protein 14, and a famesol receptor.

E21. A vector, wherein said vector comprises a polynucleotide that encodes at least one HBV peptide, wherein said vector is an adenoviral vector.

E22. A vector, wherein said vector comprises a polynucleotide that encodes at least one HBV peptide, wherein said vector is an adenoviral vector, wherein said adenoviral vector is a gorilla adenoviral vector.

E23. A polypeptide construct, wherein said polypeptide construct comprises an HBV HBx domain and at least one of an HBV Pol domain, an HBV Core domain, an HBV pre-Core domain or an HBV Surface domain.

E24. A polypeptide construct, wherein said polypeptide construct comprises a pre-Core domain and at least one of an HBV Pol domain, an HBV HBx domain or an HBV Surface domain.

E25. The polypeptide construct of E23 or E24, wherein said HBV HBx domain has a sequence as shown in SEQ ID NO: 98.

E26. The polypeptide construct of any one of E23 to E25, wherein said HBV Pol domain comprises a deletion of at least one amino acid as compared to a wildtype HBV Pol domain.

E27. The polypeptide construct of E26, wherein said deletion comprises a deleted portion of said wildtype HBV Pol domain, wherein said deleted portion comprises at least one of amino acids 538-544 or amino acids 710-742.

E28. The polypeptide construct of E27, wherein said deleted portion comprises both of amino acids 538-544 and amino acids 710-742.

E29. The polypeptide construct of E28, wherein said HBV Pol domain has a sequence as shown in SEQ ID NO: 99.

E30. The polypeptide construct of any one of E23 to E29, wherein said HBV Surface domain comprises at least one of a PreS1 domain, a PreS2 domain and an S domain.

E31. The polypeptide construct of E30, wherein said HBV Surface domain comprises an HBV S domain.

E32. The polypeptide construct of E30 or E31, wherein said HBV Surface domain has a sequence as shown in SEQ ID NO: 100.

E33. The polypeptide construct of any one of E24 to E32, wherein said polypeptide construct further comprises an HBV Core domain.

E34. The polypeptide of E23 or E33, wherein said polypeptide construct comprises a Core portion, wherein said Core portion comprises said HBV Core domain and said HBV pre-Core domain.

E35. The polypeptide construct of E34, wherein said Core portion has a sequence as shown in SEQ ID NO: 101.

E36. The polypeptide construct of E23 or E33, wherein said polypeptide construct comprises each of SHB (Env), HBeAg, HBx, and Pol domains.

E37. The polypeptide construct of E36, wherein said polypeptide construct comprises a structure, from N-terminus to C-terminus, of said SHB (Env), HBeAg, HBx, and Pol domains.

E38. The polypeptide construct of E36 or E37, wherein said SHB (Env) domain has a sequence as shown in SEQ ID NO: 102.

E39. The polypeptide construct of E36 or E37, wherein said HBeAg domain has a sequence as shown in SEQ ID NO: 103.

E40. The polypeptide construct of E36 or E37, wherein said HBx domain has a sequence as shown in SEQ ID NO: 104.

E41. The polypeptide construct of E36 or E37, wherein said Pol domain has a sequence as shown in SEQ ID NO: 105.

E42. The polypeptide construct of E36 or E37, wherein said polypeptide construct has a sequence as shown in SEQ ID NO: 106.

E43. The polypeptide construct of E36 or E37, further comprising a rigid linker.

E44. The polypeptide construct of E36 or E37, wherein said polypeptide has a sequence as shown in SEQ ID NO: 112.

E45. A polypeptide construct, wherein said polypeptide construct comprises one or more HBV HBx linkers and at least one of a Core domain, a Surface domain and a Pol domain, wherein one of said Core domain, said Surface domain and said Pol domain is connected to another of said Core domain, said Surface domain and said Pol domain by said one or more HBx linkers.

E46. The polypeptide construct of E45, wherein said Surface domain comprises at least one of an HBV PreS1 domain, an HBV PreS2 domain and an HBV S domain.

E47. The polypeptide construct of E45 or E46, wherein said one or more HBV HBx linkers comprises multiple HBV HBx linkers.

E48. The polypeptide construct of E47, wherein at least two of said multiple HBV HBx linkers differ in an amino acid sequence.

E49. The polypeptide construct of any one of E45 to E48, wherein said HBV HBx linker has a sequence as shown in any one of HBx-1, HBx-2, HBx-3, HBx-4, HBx-5 or HBx-6 of Table 3.

E50. The polypeptide construct of any one of E45 to E49, wherein said Core domain is adjacent to said Surface domain.

E51. The polypeptide construct of E50, wherein said Surface domain comprises a PreS1 domain.

E52. The polypeptide construct of E50 or E51, wherein said Surface domain is connected to said Core domain by one of said one or more HBx linkers.

E53. The polypeptide construct of any one of E45 to E52, wherein said Pol domain is adjacent to a Surface domain.

E54. The polypeptide construct of E53, wherein said Surface domain comprises at least one of a PreS1 domain, a PreS2 domain and an S domain.

E55. The polypeptide construct of E54, wherein said Surface domain comprises said PreS1 domain, and an N-terminal portion of said Pol domain is adjacent to said PreS1 domain.

E56. The polypeptide construct of E55, wherein said N-terminal portion of said Pol domain is connected to said PreS1 domain by one of said one or more HBx linkers.

E57. The polypeptide construct of E56, wherein said Surface domain comprises said PreS2 domain, and an N-terminal portion of said Pol domain is adjacent to said PreS2 domain.

E58. The polypeptide construct of E57, wherein said N-terminal portion of said Pol domain is connected to said PreS2 domain by one of said one or more HBx linkers.

E59. The polypeptide construct of E58, wherein said Surface domain comprises said PreS2 domain, and a C-terminal portion of said Pol domain is adjacent to said PreS2 domain.

E60. The polypeptide construct of E59, wherein said C-terminal portion of said Pol domain is connected to said PreS2 domain by one of said one or more HBx linkers.

E61. The polypeptide construct of E60, wherein said Surface domain comprises said S domain, and a C-terminal portion of said Pol domain is adjacent to said S domain.

E62. The polypeptide construct of E61, wherein said C-terminal portion of said Pol domain is connected to said S domain by one of said one or more HBx linkers.

E63. The polypeptide construct of any one of E45 to E62, wherein said polypeptide construct has a sequence as shown in SEQ ID NO: 107.

E64. A polypeptide construct comprising an ankyrin-like repeat domain and one or more HBV peptides.

E65. The polypeptide construct of E64, wherein said ankyrin-like repeat protein is a human ankyrin-like repeat protein.

E66. The polypeptide construct of E64 or E65, wherein said one or more HBV peptides has a sequence as shown in any one of the amino acid sequences of Table 3.

E67. The polypeptide construct of any one of E64 to E66, wherein said one or more HBV peptides comprises one or more of a Core peptide, a Surface peptide, a Pol peptide and an HBSP/HBx peptide.

E68. The polypeptide construct of E67, wherein said one or more HBV peptides comprises a Core peptide, and said Core peptide has a sequence as shown in any one of the Core amino acid sequences of Table 3.

E69. The polypeptide construct of E67, wherein said one or more HBV peptides comprises a Surface peptide, and said Surface peptide has a sequence as shown in any one of the Surface amino acid sequences of Table 3.

E70. The polypeptide construct of E67, wherein said one or more HBV peptides comprises a Pol peptide, and said Pol peptide has a sequence as shown in any one of the Pol amino acid sequences of Table 3.

E71. The polypeptide construct of E67, wherein said one or more HBV peptides comprises an HBSP/HBx peptide, and said HBSP/HBx peptide has a sequence as shown in any one of the HBSP/HBx amino acid sequences of Table 3.

E72. The polypeptide construct of any one of E65 to E71, wherein said polypeptide construct has a sequence as shown in SEQ ID NO: 108.

E73. A polypeptide construct, wherein said polypeptide construct comprises at least two HBV amino acid sequences as shown in Table 3, wherein said at least two HBV amino acid sequences are connected by a peptide linker, wherein said peptide linker is a KK linker.

E74. The polypeptide construct of E73, wherein said comprises at least two HBV amino acid sequences comprise at least one of a Core peptide, a Surface peptide, a Pol peptide and an HBSP/HBx peptide as shown in Table 3.

E75. The polypeptide construct of E73 or E74, wherein said at least two HBV amino acid sequences comprise each of the amino acid sequences as shown in Table 3.

E76. The polypeptide construct of E75, wherein said each of the amino acid sequences is connected to another of said each of the amino acid sequences by said KK linker.

E77. The polypeptide construct of any one of E73 to E75, wherein said polypeptide construct has a sequence as shown in SEQ ID NO: 109.

E78. The polypeptide construct of any one of E23 to E77 for use in a vaccine.

E79. A polynucleotide encoding the polypeptide construct of any one of E25 to E78.

E80. A vector comprising the polynucleotide of E79.

E81. The vector of E80, wherein said vector is an adenoviral vector.

E82. The vector of E81, wherein said adenoviral vector is a gorilla adenoviral vector.

### SEQUENCES

Provided herein is a representative list of certain sequences included in embodiments provided herein (Table 6).

TABLE 6
Polynucleotide/Amino Acid Sequences

| SEQ | |
|---|---|
| ID | |
| NO: | Description |
| 1 | Adenovirus pIX fragment nucleotides |
| 2 | Adenovirus DNA polymerase fragment nucleotides |
| 3 | Adenovirus penton base protein fragment nucleotides |
| 4 | Adenovirus hexon protein fragment nucleotides |
| 5 | Adenovirus fiber protein fragment nucleotides |
| 6 | Adenovirus pIX nucleotides |
| 7 | Adenovirus DNA polymerase nucleotides |
| 8 | Adenovirus penton base protein nucleotides |
| 9 | Adenovirus hexon protein nucleotides |
| 10 | Adenovirus fiber protein nucleotides |
| 11 | Adenovirus pIX protein fragment |
| 12 | Adenovirus DNA polymerase fragment |
| 13 | Adenovirus penton base protein fragment |
| 14 | Adenovirus hexon protein fragment |
| 15 | Adenovirus fiber protein fragment |
| 16 | Adenovirus pIX amino acids |
| 17 | Adenovirus DNA polymerase amino acids |
| 18 | Adenovirus penton base protein |
| 19 | Adenovirus hexon protein |
| 20 | Adenovirus fiber protein |
| 21 | Adenovirus vector nucleotide sequences |
| 22 | Adenovirus vector nucleotide sequences |
| 23 | Adenovirus vector nucleotide sequences |
| 24 | Adenovirus vector nucleotide sequences |
| 25 | Adenovirus vector nucleotide sequences |
| 26 | IL-2 core promoter |
| 27 | IL-2 minimal promoter |
| 28 | IL-2 enhancer and promoter variant |
| 29 | L-2 enhancer and promoter variant |
| 30 | (NF-κB)₁-IL2 promoter variant |
| 31 | (NF-κB)₃-IL2 promoter variant |
| 32 | (NF-κB)₆-IL2 promoter variant |
| 33 | 1X NFAT response elements-IL2 promoter variant |
| 34 | 3X NFAT response elements-IL2 promoter variant |
| 35 | 3X NFAT response elements-IL2 promoter variant |
| 36 | 6X NFAT response elements-IL2 promoter variant |
| 37 | 6X NFAT response elements-IL2 promoter variant |
| 38 | 6X NFAT response elements-IL2 promoter variant |
| 39 | 6X NFAT response elements-IL2 promoter variant |
| 40 | human EEF1A1 promoter variant |
| 41 | human EEF1A1 promoter variant |
| 42 | human EEF1A1 promoter and enhancer |
| 43 | human UBC promoter |
| 44 | synthetic minimal promoter 1 |
| 61 | GCAd-RTS-IL12 design 1 |
| 62 | GCAd-RTS-IL12 design 2 |
| 63 | GCAd-RTS-IL12 design 3 |
| 98 | HBV HBx domain of HBV design 1 |
| 99 | HBV Pol domain of HBV design 1 |
| 100 | HBV Surface (Env1) domain of HBV domain 1 |
| 101 | HBV Core domain of HBV design 1 |
| 102 | SHB(Env) domain |
| 103 | HBeAg domain |
| 104 | HBx domain |
| 105 | Pol domain |
| 106 | HBV design 1 |
| 107 | HBV design 2 |
| 108 | HBV design 3 |
| 109 | HBV design 4 |
| 112 | HBV design 5 |

1-7. (canceled)

8. A polypeptide construct comprising:
(a) an HBV HBx domain; and (ii) at least one of an HBV Pol domain, an HBV Core domain, an HBV pre-Core domain, or an HBV Surface domain; or
(b) a pre-Core domain; and (ii) at least one of an HBV Pol domain, an HBV HBx domain, or an HBV Surface domain.

9. The polypeptide construct of claim 8, wherein:
(a) the HBV HBx domain has the amino acid sequence of SEQ ID NO: 98; and or
(b) the HBV Pol domain has a deletion of at least one amino acid as compared to a wild-type HBV Pol domain.

10. The polypeptide construct of claim 8, wherein
(a) the HBV Pol domain has the amino acid sequence of SEQ ID NO: 99;
(b) the HBV Surface domain has the amino acid sequence of SEQ ID NO: 100; and/or
(c) the HBV Core domain and the HBV pre-Core domain together form a Core portion having the amino acid sequence of SEQ ID NO: 101.

11. The polypeptide construct of claim 8, comprising each of the HBV pre-Core, HBV Surface, HBx, and HBV Pol domains.

12-13. (canceled)

14. The polypeptide construct of claim 8, further comprising a rigid linker.

15. The polypeptide construct of claim 8, consisting of the amino acid sequence of SEQ ID NO: 149.

16-29. (canceled)

30. A polynucleotide encoding the polypeptide construct of claim 8.

31. A vector comprising the polynucleotide of claim 30.

32. The vector of claim 31, wherein the vector is an adenoviral vector.

33. The vector of claim 32, wherein the adenoviral vector is a gorilla adenoviral vector.

34. (canceled)

35. The polypeptide construct of claim 8 comprising:
(a) an HBV HBx domain; and
(b) at least one of an HBV Pol domain, an HBV Core domain, an HBV pre-Core domain, or an HBV Surface domain.

36. The polypeptide construct of claim 8 comprising:
(a) a pre-Core domain; and
(b) at least one of an HBV Pol domain, an HBV HBx domain, or an HBV Surface domain.

37. The polypeptide construct of claim 14, wherein the rigid linker comprises a polypeptide having the amino acid sequence of SEQ ID NO: 129.
